SEQUENCE LISTING (16 sequences)

SEQ ID NO: 1 - protein, 235 aa, Artificial Sequence
  Synthetic Polypeptide
MDFQVQIISFLLISASVIMSRGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 470 aa, Artificial Sequence
  Synthetic Polypeptide
MGWSLILLFLVAVATRVLSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 297 aa, Artificial Sequence
  Synthetic Polypeptide
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 4 - protein, 10 aa, Artificial Sequence
  Synthetic Polypeptide
  MISC_FEATURE: LC CDR1
RASSSVSYIH

SEQ ID NO: 5 - protein, 7 aa, Artificial Sequence
  Synthetic Polypeptide
  MISC_FEATURE: LC CDR2
ATSNLAS

SEQ ID NO: 6 - protein, 9 aa, Artificial Sequence
  Synthetic Polypeptide
  MISC_FEATURE: LC CDR3
QQWTSNPPT

SEQ ID NO: 7 - protein, 5 aa, Artificial Sequence
  Synthetic Polypeptide
  MISC_FEATURE: HC CDR1
SYNMH

SEQ ID NO: 8 - protein, 17 aa, Artificial Sequence
  Synthetic Polypeptide
  MISC_FEATURE: HC CDR2
AIYPGNGDTSYNQKFKG

SEQ ID NO: 9 - protein, 12 aa, Artificial Sequence
  Synthetic Polypeptide
  MISC_FEATURE: HC CDR3
STYYGGDWYFNV

SEQ ID NO: 10 - protein, 106 aa, Artificial Sequence [sequence omitted]

SEQ ID NO: 11 - protein, 121 aa, Artificial Sequence [sequence omitted]

SEQ ID NO: 12 - protein, 213 aa, Artificial Sequence
  Synthetic Polypeptide
  MISC_FEATURE: VL-IgG Kappa
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 451 aa, Artificial Sequence
  Synthetic Polypeptide
  MISC_FEATURE: VH-IgG1
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 233 aa, Artificial Sequence
  human TNF alpha
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 15 - protein, 304 aa, Artificial Sequence
  Infliximab (cA2)  light  chain variable
ASPILELELETHRGLNSERPRALAILELESERVALSERPRGLYGLARGVALSERPHESERCYSARGALASERGLNPHEVALGLYSERSERILEHISTRPTYRGLNGLNARGTHRASNGLYSERPRARGLELEILELYSTYRALASERGLSERMETSERGLYILEPRSERARGPHESERGLYSERGLYSERGLYTHRASPPHETHRLESERILEASNTHRVALGLSERGLASPILEALAASPTYRTYRCYSGLNGLNSERHISSERTRPPRPHETHRPHEGLYSERGLYTHRASNLEGLVALLYS

SEQ ID NO: 16 - protein, 340 aa, Artificial Sequence
  Infliximab (cA2) heavy chain variable
GLVALLYSLEGLGLSERGLYGLYGLYLEVALGLNPRGLYGLYSERMETLYSLESERCYSVALALASERGLYPHEILEPHESERASNHISTRPMETASNTRPVALARGGLNSERPRGLLYSGLYLEGLTRPVALALAGLILEARGSERLYSSERILEASNSERALATHRHISTYRALAGLSERVALLYSGLYARGPHETHRILESERARGASPASPSERLYSSERALAVALTYRLEGLNMETTHRASPLEARGTHRGLASPTHRGLYVALTYRTYRCYSSERARGASNTYRTYRGLYSERTHRTYRASPTYRTRPGLYGLNGLYTHRTHRLETHRVALSER

(19) United States
(12) Patent Application Publication
(10) Pub. No.: US 2022/0349898 A1
(43) Pub. Date: Nov. 3, 2022

(54) METHODS OF PRODUCING ANTIBODY COMPOSITIONS

(71) Applicant: AMGEN INC., Thousand Oaks, CA (US)

(72) Inventors: Robert J. DUFF, Moorpark, CA (US); Zhe HUANG, Acton, MA (US); Jose G. RAMIREZ, Providence, RI (US)

(73) Assignee: AMGEN INC., Thousand Oaks, CA (US)

(21) Appl. No.: 17/763,824

(22) PCT Filed: Sep. 28, 2020
(86) PCT No.: PCT/US2020/053090

§ 371 Date: Mar. 25, 2022

Related U.S. Application Data
(60) Provisional application No. 62/906,709, filed on Sep. 26, 2019.

(51) Int. Cl. G01N 33/68; G16B 5/20; C07K 16/28

(52) U.S. Cl. CPC G01N 33/6854; G16B 5/20; C07K 16/2887; C07K 2317/732; C07K 2317/41; C07K 2317/14

(57) ABSTRACT

Provided herein are methods of determining product quality of an antibody composition, wherein the ADCC activity level of the antibody composition is a criterion upon which product quality of the antibody composition is based. In exemplary embodiments, the method comprises (i) determining the total afucosylated (TAF) glycan content of a sample of an antibody composition; and (ii) determining the product quality as acceptable and/or achieving the ADCC activity level criterion when the TAF glycan content determined in (i) is within a target range. Related methods of monitoring product quality and methods of producing an antibody composition are further provided herein.

### CROSS REFERENCE TO RELATED APPLICATIONS

This application claims the benefit under 35 U.S.C. § 119(e) of U.S. Provisional Patent Application No. 62/906,709, filed on Sep. 26, 2019; the entire disclosure of which is incorporated by reference.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 26,660 byte ASCII (Text) file named “A-2451-WO-PCT_SeqList_ST25.txt”; created on Sep. 24, 2020.

### BACKGROUND

Glycosylation is one of the most common, yet important, post-translational modifications, as it plays a role in multiple cellular functions, including, for example, protein folding, quality control, molecular trafficking and sorting, and cell surface receptor interaction. Glycosylation affects the therapeutic efficacy of recombinant protein drugs, as it influences the bioactivity, pharmacokinetics, immunogenicity, solubility, and in vivo clearance of a therapeutic glycoprotein. Fc glycoform profiles, in particular, are important product quality attributes for recombinant antibodies, as they directly impact the clinical efficacy and pharmacokinetics of the antibodies.

Specific glycan structures associated with the conserved bi-antennary glycan in the Fc-CH2 domain can strongly influence the interaction with the FcγRs that mediate antibody effector functions, e.g., antibody dependent cellular cytotoxicity (ADCC) (see Reusch D, Tejada M L. Fc glycans of therapeutic antibodies as critical quality attributes. Glycobiology 2015; 25:1325-34). For example, core fucose has been demonstrated to have a very significant impact on FcγRIIIa binding affinity, leading to substantial changes in ADCC activity (see Okazaki A, et al. Fucose depletion from human IgG1 oligosaccharide enhances binding enthalpy and association rate between IgG1 and FcgammaRIIIa. Journal of molecular biology 2004; 336:1239-49; Ferrara C, et al. Unique carbohydrate-carbohydrate interactions are required for high affinity binding between FcgammaRIII and antibodies lacking core fucose. Proceedings of the National Academy of Sciences of the United States of America 2011; 108:12669-74). It has also been shown that high mannose levels also play a role in modulating ADCC activity, though to a much more modest and less predictable extent than core fucose (Thomann M, et al. Fc-galactosylation modulates antibody-dependent cellular cytotoxicity of therapeutic antibodies. Molecular immunology 2016; 73:69-75).

Different factors influence the glycan structure and thus the ultimate glycosylated form (glycoform) of the protein (glycoprotein). For example, the cell line expressing the antibody, the cell culture medium, the feed medium composition, and the timing of the feeds during cell culture can impact the production of glycoforms of the protein. While research groups have suggested many ways to influence the levels of particular glycoforms of an antibody, there still is a need in the biopharmaceutical industry for simple and efficient methods to predict the level of effector function a particular antibody composition will exhibit based on the given glycoform profile for that antibody composition. Additionally, there is a need in the art for methods of determining the levels of particular glycans, e.g., afucosylated glycans, high mannose glycans, that will achieve a desired level effector function.

### SUMMARY

The present disclosure provides methods of determining product quality of an antibody composition, wherein the ADCC activity level of the antibody composition is a criterion upon which product quality of the antibody composition is based. The method in various aspects determines the product quality in terms of the ADCC activity level criterion. In exemplary embodiments, the method comprises (i) determining the total afucosylated (TAF) glycan content of a sample of an antibody composition; and (ii) determining the product quality as acceptable and/or achieving the ADCC activity level criterion when the TAF glycan content determined in (i) is within a target range. In exemplary aspects, the target range of TAF glycan content is based on (1) a target range of ADCC activity levels for a reference antibody and (2) a first model which correlates ADCC activity level of the antibody composition to TAF glycan content of the antibody composition. In exemplary aspects, the ADCC predicted by the first model is about 95% to about 105% of the ADCC predicted by a second model, wherein the second model correlates the ADCC activity level of the antibody composition to the HM glycan content of the antibody composition and the AF glycan content of the antibody composition. As used herein, the term “predicted” in the context of ADCC activity level(s) refers to a calculated ADCC activity level, wherein the ADCC activity level is calculated according to a model, e.g., a first model, a second model. Advantageously, the ADCC predicted by the first model is statistically significantly similar to the ADCC predicted by the second model. For example, the ADCC activity level predicted by the first model is about 95% to about 105% of the ADCC activity level predicted by the second model. Optionally, the ADCC activity level predicted by the first model is about 95%, about 96%, about 97%, about 98%, about 99%, about 100%, about 101%, about 102%, about 103%, about 104%, or about 105% of the ADCC activity level predicted by the second model. The ADCC activity level predicted by the first model is, in various instances, about 100% of the ADCC predicted by the second model. In certain aspects, there is a one-to-one correspondence between the ADCC predicted by the first model and the ADCC predicted by the second model. In various instances, the first model and/or the second model is/are statistically significant. For instance, the p-value of the first model is less than 0.0001 and/or the p-value of the second model is less than 0.0001. Optionally, each of the first model and the second model has a p-value which is less than 0.0001. In exemplary aspects, the ADCC activity level predicted by the first model is ^{˜}12Q*% TAF, wherein Q is the number of antibody binding sites on the antigen to which the antibody binds and % TAF is the TAF glycan content of the antibody composition. In exemplary instances, the target range of TAF glycan content is m to n, wherein m is [ADCCₘᵢₙ/12Q], wherein ADCCₘᵢₙ is the minimum of the target range of ADCC activity level for a reference antibody, and n is [ADCCₘₐₓ]/12Q], wherein ADCCₘₐₓ is the maximum of the target range of ADCC activity level for the reference antibody. In various instances, Q is 2. In various instances, the ADCC activity level predicted by the first model is ^{˜}24*% TAF. In various instances, the target range of TAF glycan content is m to n wherein m is [ADCCₘᵢₙ/24] and n is [ADCCₘₐₓ]/24]. In various instances, the ADCC activity level predicted by the second model is ˜27*% HM+^{˜}22*% AF, wherein % AF is the AF glycan content of the antibody composition and % HM is the HM glycan content of the antibody composition. In various instances, Q is 1. In various aspects, the ADCC activity level predicted by the first model is ^{˜}12*% TAF. In various instances, the target range of TAF glycan content is m to n wherein m is [ADCCₘᵢₙ/12] and n is [ADCCₘₐₓ]/12]. In various instances, the ADCC activity level predicted by the second model is ^{˜}14.8*% HM+^{˜}12.8*% AF. Suitable alternative first models and second models are described herein. In exemplary instances, the first model is any of one of the models (e.g., equations) described herein which correlate ADCC and TAF glycan content, including but not limited to, Equations 1, 3, 5, and 7 and Equation A. In exemplary instances, the second model is any of one of the models (e.g., equations) described herein which correlate ADCC and HM glycan content and AF glycan content, including but not limited to, Equations 2, 4, 6, and 8 and Equation B. For example, in various aspects, the target range for TAF glycan content is m° to n°, wherein m° is defined as [[ADCCₘᵢₙ−y]/x], wherein ADCCₘᵢₙ is the minimum of the target range of ADCC activity level, and n° is defined as [[ADCCₘₐₓ−y]/x], wherein ADCCₘₐₓ is the maximum of the target range of ADCC activity level. Optionally, x is about 20.4 to about 27.7 and y is about −11.4 to about 16.7. Alternatively, x is about 9.7 to about 15.2 and y is about −15.6 to about 34.2. In various aspects, the target range for TAF glycan content is m′ to n′, wherein m′ is [ADCCₘᵢₙ/x′], wherein ADCCₘᵢₙ is the minimum of the target range of ADCC activity level, and n′ is [ADCCₘₐₓ]/x′], wherein ADCCₘₐₓ is the maximum of the target range of ADCC activity level. Optionally, x′ is about 24.1 to about 25.4. Alternatively, x′ is about 13.0 to about 13.95. In various instances, the ADCC activity level of the antibody composition is about 13.5%±0.5% for every 1% TAF present in the antibody composition, optionally, wherein the antibody of the antibody composition binds to an antigen comprising only one antibody binding site. In various aspects, the ADCC activity level of the antibody composition is about 24.74%±0.625% for every 1% TAF present in the antibody composition, optionally, wherein the antibody of the antibody composition binds to an antigen comprising only two antibody binding sites. In exemplary aspects, the ADCC activity level of the antibody composition is about 12%±1.5%*Q for every 1% TAF present in the antibody composition, Q is the number of antibody binding sites present on the antigen. In exemplary instances, the reference antibody is infliximab. In exemplary aspects, the reference antibody is rituximab. In exemplary aspects, the method is a quality control (QC) assay. In exemplary aspects, the method is an in-process QC assay. In various aspects, the sample is a sample of in-process material. In various instances, the TAF glycan content is determined pre-harvest or post-harvest. In exemplary instances, the TAF glycan content is determined after a chromatography step. Optionally, the chromatography step comprises a capture chromatography, intermediate chromatography, and/or polish chromatography. In some aspects, the TAF glycan content is determined after a virus inactivation and neutralization, virus filtration, or a buffer exchange. The method in various instances is a lot release assay. The sample in some aspects is a sample of a manufacturing lot. In various aspects, the method further comprises selecting the antibody composition for downstream processing, when the TAF glycan content determined in (i) is within a target range. When the TAF glycan content determined in (i) is not within the target range, one or more conditions of the cell culture are modified to obtain a modified cell culture, in various aspects. The method in some aspects, further comprises determining the TAF glycan content of a sample of the antibody composition obtained after one or more conditions of the cell culture are modified. In various aspects, when the TAF glycan content determined in (i) is not within the target range, the method further comprises (iii) modifying one or more conditions of the cell culture to obtain a modified cell culture and (iv) determining the TAF glycan content of a sample of the antibody composition obtained from the modified cell culture. In exemplary aspects, when the TAF glycan content determined in (i) is not within the target range, the method further comprises (iii) and (iv) until the TAF glycan content determined in (iv) is within the target range. In exemplary instances, an assay which directly measures ADCC activity of the antibody composition is carried out on the antibody composition only when the TAF glycan content determined in (i) is not within the target range, e.g., outside the target range. Assays which directly measure ADCC activity include for example a cell-based assay that measures the release of a detectable reagent upon lysis of antigen-expressing cells comprising the detectable agent by effector cells that are bound to antibody binding both antigen-expressing and effector cells. In exemplary instances, an assay which directly measures ADCC activity of the antibody composition is not carried out on the antibody composition. In various aspects, determining the TAF glycan content is the only step required to determine the product quality with regard to the ADCC activity level criterion. Without being bound to theory, the statistically significant correlations of the first model and the second model allow for TAF glycan content to indicate ADCC activity level such that assays that directly measure ADCC activity level are not needed. Accordingly, direct measurement of the ADCC activity level of the antibody composition is not needed and thus not carried out in various aspects of the presently disclosed methods.

The present disclosure also provides methods of monitoring product quality of an antibody composition, wherein the ADCC activity level of the antibody composition is a criterion upon which product quality of the antibody composition is based. In exemplary embodiments, the method comprises determining product quality of an antibody composition in accordance with a method of the present disclosures, with a first sample obtained at a first timepoint and with a second sample taken at a second timepoint which is different from the first timepoint. In various instances, each of the first sample and second sample is a sample of in-process material. In various aspects, the first sample is a sample of in-process material and the second sample is a sample of a manufacturing lot. Optionally, the first sample is a sample obtained before one or more conditions of the cell culture are modified and the second sample is a sample obtained after the one or more conditions of the cell culture are modified. In exemplary instances, the TAF glycan content is determined for each of the first sample and second sample. Product quality of the antibody composition depends on whether the TAF glycan content is within a target range. In exemplary aspects, the target range of TAF glycan content is based on (1) a target range of ADCC activity levels for a reference antibody and (2) a first model which correlates ADCC activity level of the antibody composition to TAF glycan content of the antibody composition. In exemplary aspects, the ADCC predicted by the first model is about 95% to about 105% of the ADCC predicted by a second model, wherein the second model correlates the ADCC activity level of the antibody composition to the HM glycan content of the antibody composition and the AF glycan content of the antibody composition.

The present disclosure provides methods of producing an antibody composition. In exemplary embodiments, the method comprises determining product quality of the antibody composition wherein product quality of the antibody composition is determined in accordance with a method of the present disclosures. Optionally, the method comprises determining the TAF glycan content of a sample of an antibody composition and the sample is a sample of in-process material. In various instances, the method comprises determining the product quality of the antibody composition as acceptable and/or achieving the ADCC activity level criterion when the TAF glycan content determined in (i) is within a target range, as defined herein. In exemplary aspects, the target range of TAF glycan content is based on (1) a target range of ADCC activity levels for a reference antibody and (2) a first model which correlates ADCC activity level of the antibody composition to TAF glycan content of the antibody composition. In exemplary aspects, the ADCC predicted by the first model is about 95% to about 105% of the ADCC predicted by a second model, wherein the second model correlates the ADCC activity level of the antibody composition to the HM glycan content of the antibody composition and the AF glycan content of the antibody composition. In various aspects, when the TAF glycan content determined in (i) is not within the target range, the method further comprises (iii) modifying one or more conditions of the cell culture to obtain a modified cell culture and (iv) determining the TAF glycan content of a sample of the antibody composition obtained from the modified cell culture, optionally, repeating steps (iii) and (iv) until the TAF glycan content is within the target range. in various instances, the sample is a sample of a cell culture comprising cells expressing an antibody of the antibody composition. In various instances, one or more conditions of the cell culture are modified to modify the TAF glycan content. In various aspects, the TAF glycan content of the antibody composition is achieved by modifying the AF glycan content. In exemplary aspects, one or more conditions of the cell culture are modified to modify the AF glycan content of the antibody composition. In exemplary aspects, the one or more conditions primarily modify the AF glycan content. In various instances, the one or more conditions modify the AF glycan content and does not modify the HM glycan content. In exemplary aspects, the method comprises the TAF glycan content of the antibody composition is achieved by modifying the HM glycan content. Optionally, one or more conditions of the cell culture are modified to modify the HM glycan content of the antibody composition. In some instances, the one or more conditions primarily modify the HM glycan content. In some aspects, the one or more conditions modify the HM glycan content and does not modify the AF glycan content. In various instances, the method comprises repeating the modifying of the afucosylated (AF) glycan content and/or repeating the modifying of the high mannose (HM) glycan, until the TAF glycan content is within a target range.

In exemplary embodiments, the method of producing an antibody composition comprises (i) determining the TAF glycan content of a sample of an antibody composition; and (ii) selecting the antibody composition for downstream processing based on the TAF glycan content determined in (i). In various aspects, the sample is taken from a cell culture comprising cells expressing an antibody of the antibody composition. In various instances, the method further comprises modifying the TAF glycan content of the antibody composition and determining the modified TAF glycan content. Optionally, one or more conditions of the cell culture are modified in order to modify the TAF glycan content. In exemplary aspects, the method comprises repeating the modifying until the TAF glycan content is within a target range. In exemplary instances, the target range is based on a target range of ADCC activity level for the antibody. Without being bound to theory, the TAF glycan content correlates with the ADCC activity level of the antibody composition such that the ADCC activity level of an antibody composition may be predicted based on the TAF glycan content of the antibody composition. The ADCC activity level of the antibody composition may be a criteria worth considering when deciding whether the antibody composition should be selected for downstream processing. Therefore, in various aspects, the method comprises (i) determining the TAF glycan content of a sample of an antibody composition; (ii) determining the ADCC activity level of the antibody composition based on the TAF glycan content determined in (i), and, optionally, (iii) selecting the antibody composition for downstream processing when the ADCC level of the antibody composition determined in (ii) is within a target range of ADCC activity level. In various aspects, the target range of ADCC activity level is known for the antibody of the antibody composition. The antibody of the antibody composition, in various aspects, is a biosimilar of a reference antibody. In various instances, a target range of TAF glycan content is based or determined (e.g., calculated) based on the target range of ADCC activity level which is known. Accordingly, in exemplary aspects, the method comprises (i) determining the TAF glycan content of a sample of an antibody composition; and (ii) selecting the antibody composition for downstream processing when the TAF glycan content determined in (i) is within a target range. When the method further comprises modifying the TAF glycan content of the antibody composition, the method in various instances comprises modifying the afucosylated (AF) glycan content to modify the TAF glycan content. Optionally, one or more conditions of the cell culture are modified to modify the AF glycan content of the antibody composition, which, in turn, modifies the TAF glycan content. Alternatively or additionally, when the method further comprises modifying the TAF glycan content of the antibody composition, the method in various instances comprises modifying the high mannose (HM) glycan content to modify the TAF glycan content. Optionally, one or more conditions of the cell culture are modified to modify the HF glycan content of the antibody composition, which, in turn, modifies the TAF glycan content. In exemplary aspects, the one or more conditions primarily modify the AF glycan content. In exemplary instances, the one or more conditions primarily modify the HM glycan content. In exemplary aspects, the one or more conditions modify the AF glycan content and not the HM glycan content. In exemplary instances, the one or more conditions modify the HM glycan content and not the AF glycan content. The method optionally comprises repeating the modifying of the afucosylated (AF) glycan content and/or repeating the modifying of the high mannose (HM) glycan, until the TAF glycan content is within a target range. In exemplary aspects, the antibody of the antibody composition is an IgG, optionally, an IgG₁. In various aspects, the target range for TAF glycan content is m to n, wherein m is [[ADCCₘᵢₙ−y]/x], wherein ADCCₘᵢₙ is the minimum of the target range of ADCC activity level, and n is [[ADCCₘₐₓ−y]/x], wherein ADCCₘₐₓ is the maximum of the target range of ADCC activity level. Optionally, x is about 20.4 to about 27.7 and y is about −11.4 to about 16.7. Alternatively, x is about 9.7 to about 15.2 and y is about −15.6 to about 34.2. In various aspects, the target range for TAF glycan content is m′ to n′, wherein m′ is [ADCCₘᵢₙ/x′], wherein ADCCₘᵢₙ is the minimum of the target range of ADCC activity level, and n′ is [ADCCₘₐₓ]/x′], wherein ADCCₘₐₓ is the maximum of the target range of ADCC activity level. Optionally, x′ is about 24.1 to about 25.4. Alternatively, x′ is about 13.0 to about 13.95. In various instances, the ADCC activity level of the antibody composition is about 13.5%±0.5% for every 1% TAF present in the antibody composition, optionally, wherein the antibody of the antibody composition binds to an antigen comprising only one antibody binding site. In various aspects, the ADCC activity level of the antibody composition is about 24.74%±0.625% for every 1% TAF present in the antibody composition, optionally, wherein the antibody of the antibody composition binds to an antigen comprising only two antibody binding sites. In exemplary aspects, the ADCC activity level of the antibody composition is about 12%±1.5%*Q for every 1% TAF present in the antibody composition, Q is the number of antibody binding sites present on the antigen. In exemplary instances, Q is 1 and optionally the antibody is infliximab or a biosimilar thereof. Optionally, Q is 2 and optionally the antibody is rituximab or a biosimilar thereof.

In exemplary embodiments, the method of producing an antibody composition comprises (i) determining the % total afucosylated (TAF) glycans of an antibody composition; (ii) calculating a % antibody dependent cellular cytotoxicity (ADCC) of the antibody composition based on the % TAF using Equation A:

Y=2.6+24.1*X [Equation A],
wherein Y is the % ADCC and X is the % TAF glycans determined in step (i),

and (iii) selecting the antibody composition for one or more downstream processing steps when Y is within a target % ADCC range.

The present disclosure also provides a method of producing an antibody composition, wherein, the method comprises (i) determining the % high mannose glycans and the % afucosylated glycans of an antibody composition; (ii) calculating a % antibody dependent cellular cytotoxicity (ADCC) of the antibody composition based on the % high mannose glycans and the % afucosylated glycans using Equation B:

Y=(0.24+27*HM+22.1*AF) [Equation B],
wherein Y is the % ADCC, HM is the % high mannose glycans determined in step (i), and AF is the % afucosylated glycans determined in step (i),

and (iii) selecting the antibody composition for one or more downstream processing steps when Y is within a target % ADCC range.

The present disclosure additionally provides methods of producing an antibody composition with a target % ADCC. In exemplary embodiments, the method comprises (i) calculating a target % total afucosylated (TAF) glycans for the target % ADCC using Equation A:

Y=2.6+24.1*X [Equation A],

wherein Y is the target % ADCC and X is the target % TAF glycans,

and (ii) maintaining glycosylation-competent cells in a cell culture to produce an antibody composition with the target % TAF glycans, X.

The present disclosure further provides methods of producing an antibody composition with a target % ADCC, wherein the method comprises (i) calculating a target % afucosylated glycans and a target % high mannose glycans for the target % ADCC using Equation B:

Y=(0.24+27*HM+22.1*AF) [Equation B],
wherein Y is the target % ADCC, HM is the target % high mannose glycans and AF is the target % afucosylated glycans
   and (ii) maintaining glycosylation-competent cells in a cell culture to produce an antibody composition with the target % high mannose glycans and the target % afucosylated glycans.

In exemplary aspects of the presently disclosed methods, the target % ADCC is within a target % ADCC range. Optionally, the target % ADCC range is greater than or about 40 and less than or about 170. In various aspects, the target % ADCC range is greater than or about 44 and less than or about 165. In various instances, the target % ADCC range is greater than or about 60 and less than or about 130. In exemplary aspects, the target % ADCC range is Y±20, e.g., Y±17 or Y±18.

Further provided are methods of producing an antibody composition with a % ADCC, Y, which is optionally greater than or about 40 and less than or about 170, said method comprising (i) determining the % total afucosylated (TAF) glycans, X, of the antibody composition, and (ii) selecting the antibody composition for one or more downstream processing steps, when X is equivalent to (Y−2.6)/24.1. In exemplary aspects, X is greater than or about 1.55% and less than or about 6.95%. In various aspects, Y is greater than or about 44% and less than or about 165%, and optionally, wherein X is about 1.72% to about 6.74%.

The present disclosure provides method of producing an antibody composition with a % ADCC, Y, said method comprising (i) determining the % total afucosylated (TAF) glycans, X, of the antibody composition, and (ii) selecting the antibody composition for one or more downstream processing steps, when the X is equivalent to (Y−2.6)/24.1, optionally, wherein X is greater than or about X−0.4 and less than or about X+0.4, and wherein the % ADCC is greater than about Y−17 and less than or about Y+17.

Also provided is a method of producing an antibody composition with a % ADCC, said method comprising (i) determining the % afucosylated glycans and the % high mannose glycans of the antibody composition, and and (ii) selecting the antibody composition for one or more downstream processing steps, when AF and HM are related to Y according to Equation B

Y=(0.24+27*HM+22.1*AF) [Equation B],

wherein Y is the % ADCC, HM is the % high mannose glycans determined in step (i), and AF is the afucosylated glycans determined in step (i). In exemplary aspects, Y is greater than or about 40 and less than or about 175, optionally, about 41 to about 171, wherein AF is about 1 to about 4 and wherein HM is about 40 to about 175. Optionally, Y is about 30 to about 185, optionally, about 32 to about 180, wherein HM is about 1 to about 4 and wherein AF is about 30 to about 185. In exemplary instances, the % ADCC of the antibody composition is within a range defined by Y. Optionally, the % ADCC of the antibody composition is within a range of Y±18. In exemplary aspects, AF is about 1 to about 4. Optionally, the % high mannose glycans is a value within a range defined by HMI, optionally, wherein the range is HM±1. In various instances, HM is about 1 to about 4. Optionally, the % afucosylated glycans is a value within a range defined by AF optionally, wherein the range is AF±1.

In exemplary embodiments, the presently disclosed methods of producing an antibody composition comprises modifying total afucosylated (TAF) glycan content of an antibody composition produced by cells of a cell culture. In various instances, one or more conditions of the cell culture are modified to modify the TAF glycan content. In various aspects, the method comprises determining the modified TAF glycan content. Optionally, the modifying is repeated until the determined TAF glycan content is in a target range of TAF. Without being bound to a particular theory, the TAF glycan content may be modified by changing the afucosylated (AF) glycan content or the high mannose (HM) content, or a combination thereof, since each impacts the TAF glycan content. Accordingly, the methods advantageously allow for multiple ways to achieve the target range of TAF glycan content. For example, one or more conditions of the cell culture are modified to modify the AF glycan content in order to modify the TAF glycan content. Alternatively, one or more conditions of the cell culture are modified to modify the HM glycan content in order to modify the TAF glycan content. In various instances, one or more conditions of the cell culture are modified to modify the AF glycan content and the HM glycan content in order to modify the TAF glycan content. Therefore, the present disclosure further provides methods of modifying total afucosylated (TAF) glycan content of an antibody composition produced by cells of a cell culture. In exemplary embodiments, the method comprises modifying the AF glycan content. In exemplary embodiments, the method comprises modifying the HM glycan content. In various aspects, the method comprises (i) determining the afucosylated (AF) glycan content and the high mannose (HM) glycan content of a sample of an antibody composition; (ii) determining a target range of AF glycan content based on a target range of ADCC activity level of an antibody of the antibody composition, assuming the HM glycan content is constant; and (iii) selecting the antibody composition for downstream processing when the AF glycan content is in the target range of AF glycan content. In various instances, the method comprises (i) determining the afucosylated (AF) glycan content and the high mannose (HM) glycan content of a sample of an antibody composition; (ii) determining a target range of HM glycan content based on a target range of ADCC activity level of an antibody of the antibody composition, assuming the AF glycan content is constant; and (iii) selecting the antibody composition for downstream processing when the HM glycan content is in the target range of AF glycan content. In various instances, the method comprises (i) determining the AF glycan content and the HM glycan content of a sample of the antibody composition and (ii) determining a target range of AF glycan content based on the HM glycan content determined in (i), and (iii) modifying the AF glycan content until it is within the target range of AF glycan content, wherein the HM glycan content is unmodified. Alternatively, the method comprises (i) determining the AF glycan content and the HM glycan content of a sample of the antibody composition and (ii) determining a target range of HM glycan content based on the AF glycan content determined in (i), and (iii) modifying the HM glycan content until it is within the target range of HM glycan content, wherein the AF glycan content is unmodified. In exemplary aspects, the model which correlates ADCC activity level of the antibody composition to the TAF glycan content of the antibody composition predicts essentially the same ADCC activity level predicted by the model which correlates ADCC to HM and AF glycan content.

In various aspects of the presently disclosed methods, the % TAF glycans is determined by calculating the sum of the % high mannose glycans and the % afucosylated glycans. In various instances, the % high mannose glycans and the % afucosylated glycans are determined by hydrophilic interaction chromatography. Optionally, the % high mannose glycans and the % afucosylated glycans are determined by the method described in Example 1. In various aspects, the % ADCC is determined by a quantitative cell-based assay which measures the ability of the antibodies of the antibody composition to mediate cell cytotoxicity in a dose-dependent manner in cells expressing the antigen of the antibodies and engaging Fc-gammaRIIIA receptors on effector cells through the Fc domain of the antibodies. In various instances, the % ADCC is determined by the assay described in Example 2. In exemplary aspects, the determining step is carried out after a harvest step. Optionally, the determining step is carried out after a chromatography step. In various aspects, the chromatography step is a Protein A chromatography step. In various instances of the presently disclosed methods, the one or more downstream processing steps comprise(s): a dilution step, a filling step, a filtration step, a formulation step, a chromatography step, a viral filtration step, a viral inactivation step, or a combination thereof. Optionally, the chromatography step is an ion exchange chromatography step, optionally, a cation exchange chromatography step or an anion exchange chromatography step.

In various aspects of the present disclosure, each antibody of the antibody composition is an IgG, optionally, each antibody of the antibody composition is an IgG₁. In exemplary instances, each antibody of the antibody composition binds to a tumor-associated antigen. In exemplary aspects, the tumor-associated antigen comprises the amino acid sequence of SEQ ID NO. 3. In exemplary aspects, each antibody of the antibody composition is an anti-CD20 antibody. In various instances, each antibody of the antibody composition comprises: (i) a light chain (LC) CDR1 comprising an amino acid sequence of SEQ ID NO: 4 or an amino acid sequence which is at least 90% identical to SEQ ID NO: 4 or a variant amino acid sequence of SEQ ID NO: 4 with 1 or 2 amino acid substitutions, (ii) a LC CDR2 comprising an amino acid sequence of SEQ ID NO: 5 or an amino acid sequence which is at least 90% identical to SEQ ID NO: 5 or a variant amino acid sequence of SEQ ID NO: 5 with 1 or 2 amino acid substitutions, (iii) a LC CDR3 comprising an amino acid sequence of SEQ ID NO: 6 or an amino acid sequence which is at least 90% identical to SEQ ID NO: 6 or a variant amino acid sequence of SEQ ID NO: 6 with 1 or 2 amino acid substitutions, (iv) a heavy chain (HC) CDR1 comprising an amino acid sequence of SEQ ID NO: 7 or an amino acid sequence which is at least 90% identical to SEQ ID NO: 7 or a variant amino acid sequence of SEQ ID NO: 7 with 1 or 2 amino acid substitutions; (v) a HC CDR2 comprising an amino acid sequence of SEQ ID NO: 8 or an amino acid sequence which is at least 90% identical to SEQ ID NO: 8 or a variant amino acid sequence of SEQ ID NO: 8 with 1 or 2 amino acid substitutions; and/or (vi) a HC CDR3 comprising an amino acid sequence of SEQ ID NO: 9 or an amino acid sequence which is at least 90% identical to SEQ ID NO: 9 or a variant amino acid sequence of SEQ ID NO: 9 with 1 or 2 amino acid substitutions.

In exemplary aspects, each antibody of the antibody composition comprises a LC variable region comprising an amino acid sequence of SEQ ID NO: 10, an amino acid sequence which is at least 90% identical to SEQ ID NO: 10, or a variant amino acid sequence of SEQ ID NO: 10 with 1 to 10 amino acid substitutions. Optionally, each antibody of the antibody composition comprises a HC variable region comprising an amino acid sequence of SEQ ID NO: 11, an amino acid sequence which is at least 90% identical to SEQ ID NO: 11, or a variant amino acid sequence of SEQ ID NO: 11 with 1 to 10 amino acid substitutions. In exemplary aspects, each antibody of the antibody composition comprises a light chain comprising an amino acid sequence of SEQ ID NO: 12, an amino acid sequence which is at least 90% identical to SEQ ID NO: 12, or a variant amino acid sequence of SEQ ID NO: 12 with 1 to 10 amino acid substitutions. In exemplary instances, each antibody of the antibody composition comprises a heavy chain comprising an amino acid sequence of SEQ ID NO: 13, an amino acid sequence which is at least 90% identical to SEQ ID NO: 13, or a variant amino acid sequence of SEQ ID NO: 13 with 1 to 10 amino acid substitutions.

In exemplary aspects, the tumor-associated antigen comprises the amino acid sequence of SEQ ID NO. 14. In exemplary aspects, each antibody of the antibody composition is an anti-TNFα antibody, optionally, infliximab or a biosimilar thereof. In exemplary aspects, each antibody of the antibody composition comprises a LC variable region comprising an amino acid sequence of SEQ ID NO: 15, an amino acid sequence which is at least 90% identical to SEQ ID NO: 15, or a variant amino acid sequence of SEQ ID NO: 15 with 1 to 10 amino acid substitutions. Optionally, each antibody of the antibody composition comprises a HC variable region comprising an amino acid sequence of SEQ ID NO: 16, an amino acid sequence which is at least 90% identical to SEQ ID NO: 16, or a variant amino acid sequence of SEQ ID NO: 16 with 1 to 10 amino acid substitutions.

The present disclosure further provides methods of producing an antibody composition within a target % ADCC range said method comprises: (i) measuring the % ADCC of a series of samples comprising varying glycoforms of an antibody, (ii) determining the % total afucosylated (TAF) glycans for each sample of the series, (iii) determining a linear equation of a best fit line of a graph which plots for each sample of the series the % ADCC as measured in step (i) as a function of the % TAF glycans as determined in step (ii), (iv) determining the % TAF for an antibody composition and then calculating a % ADCC using the linear equation of step (iii), and (v) selecting the antibody composition for one or more downstream processing steps when the % ADCC calculated in step (iv) is within a target % ADCC range.

A method of producing an antibody composition within a target range of TAF glycan content is provided wherein said method comprises: (i) measuring the ADCC activity level of a series of samples comprising varying glycoforms of an antibody, (ii) determining the TAF glycan content for each sample of the series, (iii) creating a model which correlates the ADCC activity level to the TAF glycan content, (iv) determining the ADCC activity level for an antibody composition and then calculating a TAF glycan content using the model or determining the TAF glycan content for the antibody composition and calculating the ADCC activity level using the model, and (v) selecting the antibody composition for one or more downstream processing steps when the TAF glycan content calculated in step (iv) is within a target range of TAF glycan content or when the ADCC activity level calculated in step (iv) is within a target range of ADCC activity level.

A method of producing an antibody composition within a target % TAF range is provided wherein said method comprises: (i) measuring the % ADCC of a series of samples comprising varying glycoforms of an antibody, (ii) determining the % total afucosylated (TAF) glycans for each sample of the series, (iii) determining a linear equation of a best fit line of a graph which plots for each sample of the series the % ADCC as measured in step (i) as a function of the % TAF glycans as determined in step (ii), (iv) determining a linear equation of a best fit line of a graph which plots for each sample of the series the % ADCC as measured in step (i) as a function of the % TAF glycans as determined in step (ii), (v) determining the % ADCC for an antibody composition and then calculating a % TAF using the linear equation of step (iii), and (iv) selecting the antibody composition for one or more downstream processing steps when the % TAF calculated in step (iv) is within a target % TAF range. Also provided is a method of producing an antibody composition within a target % TAF range wherein the method comprises the following steps: (i) generating a linear equation of a best fit graph by plotting the % ADCC and % TAF glycans of a series of at least 5 reference antibody compositions produced under cell culture conditions, each reference antibody composition having the same amino acid sequence as the antibody composition, (ii) selecting a target % TAF glycan range based on the linear equation generated in step (i) and desired % ADCC activity; (iii) culturing the antibody composition under cell culture conditions; (iv) purifying the antibody composition, (v) sampling the antibody composition to determine the % TAF and (vi) determining whether the % TAF of the antibody composition is within the target % TAF range of step (ii). In exemplary aspects, the method further comprises selecting the antibody composition for one or more downstream processing steps when the % TAF calculated in step (v) is within the target % TAF range.

Also provided is a method of determining % antibody dependent cellular cytotoxicity (ADCC) of an antibody composition, said method comprising: (i) determining the % total afucosylated (TAF) glycans of an antibody composition; and (ii) calculating the % ADCC of the antibody composition based on the % TAF using Equation A

Y=2.6+24.1*X [Equation A],
wherein Y is the % ADCC and X is the % TAF glycans determined in step (i),

Additionally, a method of determining % antibody dependent cellular cytotoxicity (ADCC) of an antibody composition, is provided, said method comprising (i) determining the % high mannose glycans and the % afucosylated glycans of an antibody composition, and (ii) calculating the % ADCC of the antibody composition based on the % high mannose glycans and the % afucosylated glycans using Equation B:

Y=(0.24+27*HM+22.1*AF) [Equation B],
wherein Y is the % ADCC, HM is the % high mannose glycans determined in step (i),
and AF is the % afucosylated glycans determined in step (i).

In exemplary instances, the methods further comprise selecting the antibody composition for one or more downstream processing steps when Y is within a target % ADCC range.

### BRIEF DESCRIPTION OF THE DRAWINGS

FIG. 1A is an illustration of the three types of N-glycans (oligomannose, complex and hybrid) and commonly used symbols for such saccharides. FIG. 1B is an illustration of exemplary glycan structures.

FIG. 2A is a representative glycan map chromatogram (full scale view). FIG. 2B is a representative glycan map chromatogram (expanded scale view).

FIG. 3 is a schematic of the NK92 ADCC assay described in Example 2.

FIG. 4 is a representative dose-response curve for the NK92 ADCC Assay. Each dose point is a mean±standard deviation of 3 replicates. Assay signal=fluorescence

FIG. 5A is a graph of actual ADCC (%) plotted as a function of TAF (%). The best fit line is shown. FIG. 5B is a table of statistical parameters of the best fit line of FIG. 5A. FIG. 5C is a graph of the actual ADCC (%) (as determined by the assay described in Example 2) plotted as a function of predicted ADCC (%) as calculated using the prediction expression equation shown in FIG. 5B. FIG. 5D is the graph of FIG. 5A showing the 95% confidence band (shaded grey). FIG. 5E provides a graph of the 95% confidence region for both the y-intercept and slope of Equation 1.

FIG. 6A is a graph of actual ADCC (%) plotted as a function of HM (%). The best fit line is shown. FIG. 6B is a graph of actual ADCC (%) plotted as a function of AF (%). The best fit line is shown. FIG. 6C a table of statistical parameters of the best fit line(s) shown in FIGS. 6A and 6B.

FIG. 6D is a graph of the actual ADCC (%) (as determined by the assay described in Example 2) plotted as a function of predicted ADCC (%) as calculated using the prediction expression equation shown in FIG. 4C.

FIG. 7A is a graph of actual ADCC (%) plotted as a function of galactosylation (%). The best fit line is shown in red. FIG. 7B is a graph of the actual ADCC (%) (as determined by the assay described in Example 2) plotted as a function of predicted ADCC (%) as calculated using a prediction expression equation correlating ADCC and galactosylation (not shown).

FIG. 8A is a graph of actual ADCC (%) plotted as a function of TAF (%). The best fit line is shown. FIG. 8B is a table of statistical parameters of the best fit line of FIG. 8A. FIG. 8C is a graph of the actual ADCC (%) (as determined by the assay described in Example 2) plotted as a function of predicted ADCC (%) as calculated using the prediction expression equation shown in FIG. 8B. FIG. 8D is the graph of FIG. 8A showing the 95% confidence band (shaded grey). FIG. 8E provides a graph of the 95% confidence region for both the y-intercept and slope of Equation 3.

FIG. 9A is a graph of actual ADCC (%) plotted as a function of HM (%). The best fit line is shown. FIG. 9B is a graph of actual ADCC (%) plotted as a function of AF (%). The best fit line is shown. FIG. 9C a table of statistical parameters of the best fit line(s) shown in FIGS. 9A and 9B. FIG. 9D is a graph of the actual ADCC (%) (as determined by the assay described in Example 2) plotted as a function of predicted ADCC (%) as calculated using the prediction expression equation shown in FIG. 9C.

FIG. 10A and FIG. 10B are graphs correlating the no y-intercept predictions of the ADCC-HM/AF model to the no y-intercept predictions of the ADCC-TAF model for the anti-CD20 antibody (FIG. 10A) and for the anti-TNFalpha antibody (FIG. 10B).

### DETAILED DESCRIPTION

Provided herein for the first time are data demonstrating a statistically significant association between the ADCC level of an antibody composition and the level of TAF glycans of that antibody composition. Also provided herein for the first time are data demonstrating a statistically significant association between the ADCC level of an antibody composition and the level of high mannose glycans and afucosylated glycans of that antibody composition. As further described herein, Equation A and Equation B, associate % ADCC of an antibody composition with the % TAF glycans (Equation A) or with the % high mannose glycans and % afucosylated glycans (Equation B) of the antibody composition. These associations and equations and others of the present disclosure are useful in methods for predicting the level of ADCC of an antibody composition based on the levels of the glycans. In various aspects, the predicted ADCC level serves as a marker by which an antibody composition is identified as acceptable in terms of meeting a therapeutic threshold, and thus is one which should be used in one or more downstream manufacturing process steps, or, alternatively, the antibody composition is identified as unacceptable and should not be carried forward in the manufacturing process. The presently disclosed associations and equations are further useful in identifying the glycoprofile of desired antibody compositions. With the associations and equations presented herein, and given a target ADCC level, the glycoprofile (e.g., profile of TAF glycans, HM glycans, afucosylated glycans) of antibody compositions with the target ADCC level are identified. With the identified profile of TAF glycans, HM glycans, afucosylated glycans of antibody compositions with the target ADCC level, manufacturing processes, e.g., cell culturing steps, may be carried out to target that identified profile.

Accordingly, the present disclosure provides methods of determining product quality of an antibody composition, wherein at least one of the acceptance criteria for the antibody composition is ADCC activity level. Methods of monitoring product quality of an antibody composition are also provided. The present disclosure further provides methods of producing an antibody composition, e.g., methods of producing an antibody composition with a target % ADCC, methods of producing an antibody composition with a % ADCC within a target % ADCC range or with an identified % ADCC, and methods of producing an antibody composition within a target % TAF range, are provided herein.

Glycosylation, Glycans, and Methods of Glycan Measurement

Many secreted proteins undergo post-translational glycosylation, a process by which sugar moieties (e.g., glycans, saccharides) are covalently attached to specific amino acids of a protein. In eukaryotic cells, two types of glycosylation reactions occur: (1) N-linked glycosylation, in which glycans are attached to the asparagine of the recognition sequence Asn-X-Thr/Ser, where “X” is any amino acid except proline, and (2) O-linked glycosylation in which glycans are attached to serine or threonine. Regardless of the glycosylation type (N-linked or O-linked), microheterogeneity of protein glycoforms exists due to the large range of glycan structures associated with each site (O or N).

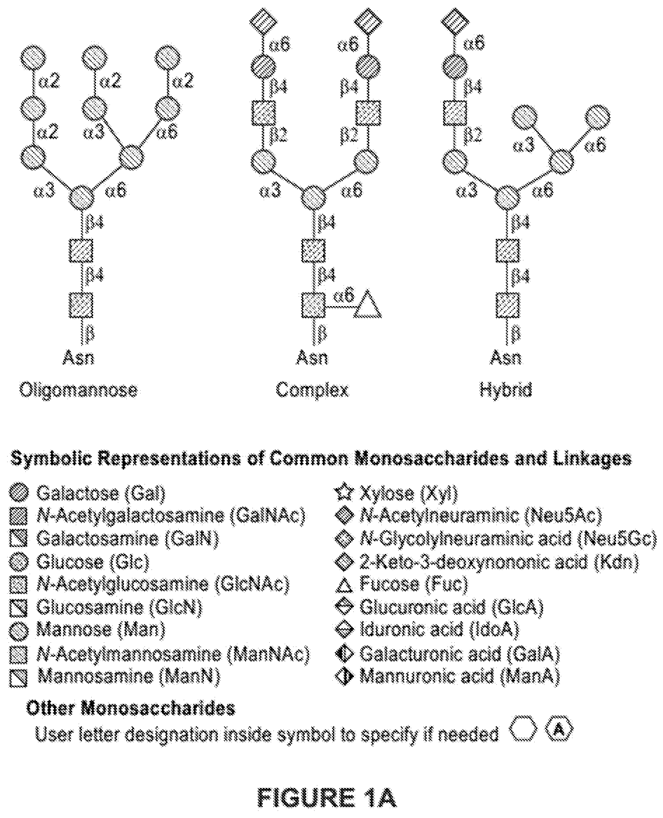

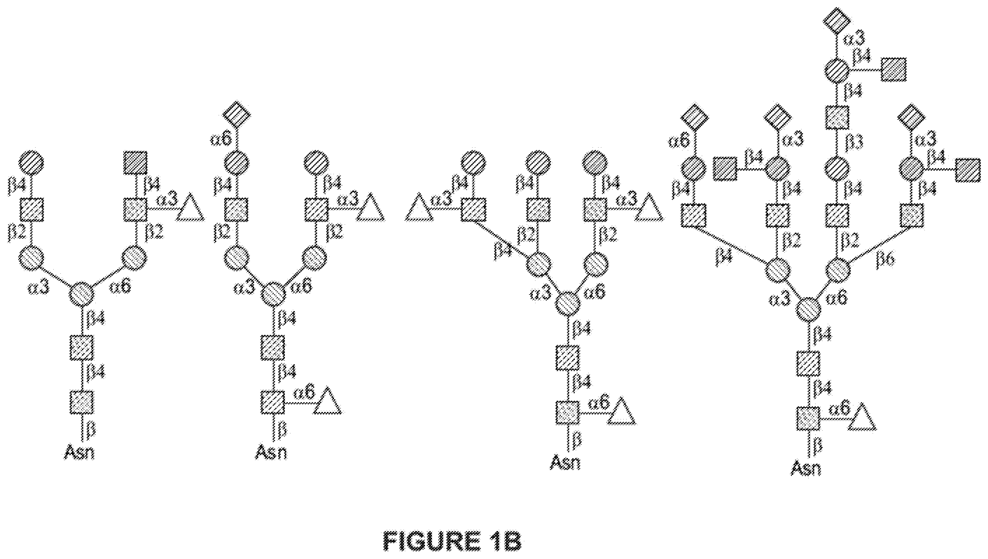

All N-glycans have a common core sugar sequence: Manα1-6(Manα1-3)Manβ1-4GlcNAcβ1-4GlcNAcβ1-Asn-X-Ser/Thr (Man₃GlcNAc₂Asn) and are categorized into one of three types: (A) a high mannose (HM) or oligomannose (OM) type, which consists of two N-acetylglucosamine (GalNAc) moieties and a large number (e.g., 5, 6, 7, 8 or 9) of mannose (Man) residues (B) a complex type, which comprises more than two GlcNAc moieties and any number of other sugar types or (C) a hybrid type, which comprises a Man residue on one side of the branch and GlcNAc at the base of a complex branch. FIG. 1A (taken from Stanley et al., Chapter 8: N-Glycans, Essentials of Glycobiology, 2^{nd} ed., Cold Spring Harbor Laboratory Press; 2009) shows the three types of N-glycans.

N-linked glycans typically comprise one or more monosaccharides of galactose (Gal), N-acetylgalactosamine (GalNAc), galactosamine (GalN), glucose (GLc), N-acetylglucoasamine (ClcNAc), glucoasamine (GlcN), mannose (Man), N-Acetylmannosamine (ManNAc), Mannosamine (ManN), xylose (Xyl), N-Acetylneuraminic acid (Neu5Ac), N-Glycolylneuraminic acid (Neu5Gc), 2-keto-3-doxynononic acid (Kdn), fucose (Fuc), Glucuronic acid (GLcA), Iduronic acid (IdoA), Galacturonic acid (Gal A), mannuronic acid (Man A). The commonly used symbols for such saccharides are shown in FIG. 1A. Exemplary glycans and their identity are shown in FIG. 1B.

N-linked glycosylation begins in the endoplasmic reticulum (ER), where a complex set of reactions result in the attachment of a core glycan structure made essentially of two GlcNAc residues and three Man residues. The glycan complex formed in the ER is modified by action of enzymes in the Golgi apparatus. If the saccharide is relatively inaccessible to the enzymes, it typically stays in the original HM form. If enzymes can access the saccharide, then many of the Man residues are cleaved off and the saccharide is further modified, resulting in the complex type N-glycans structure. For example, mannosidase-1 located in the cis-Golgi, can cleave or hydrolyze a HM glycan, while fucosyltransferase FUT-8, located in the medial-Golgi, fucosylates the glycan (Hanrue Imai-Nishiya (2007), BMC Biotechnology, 7:84).

Accordingly, the sugar composition and the structural configuration of a glycan structure varies, depending on the glycosylation machinery in the ER and the Golgi apparatus, the accessibility of the machinery enzymes to the glycan structure, the order of action of each enzyme and the stage at which the protein is released from the glycosylation machinery, among other factors.

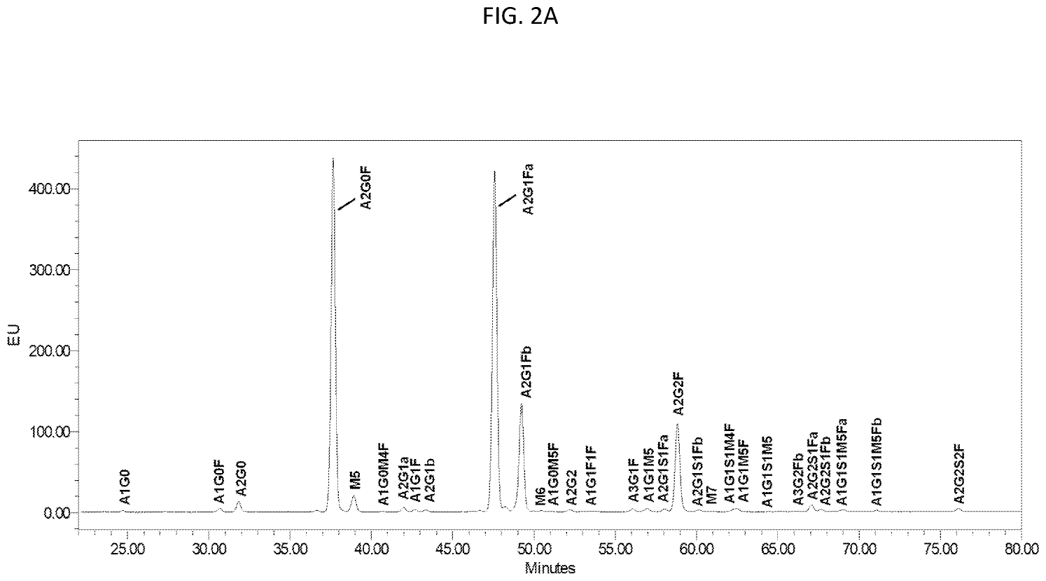

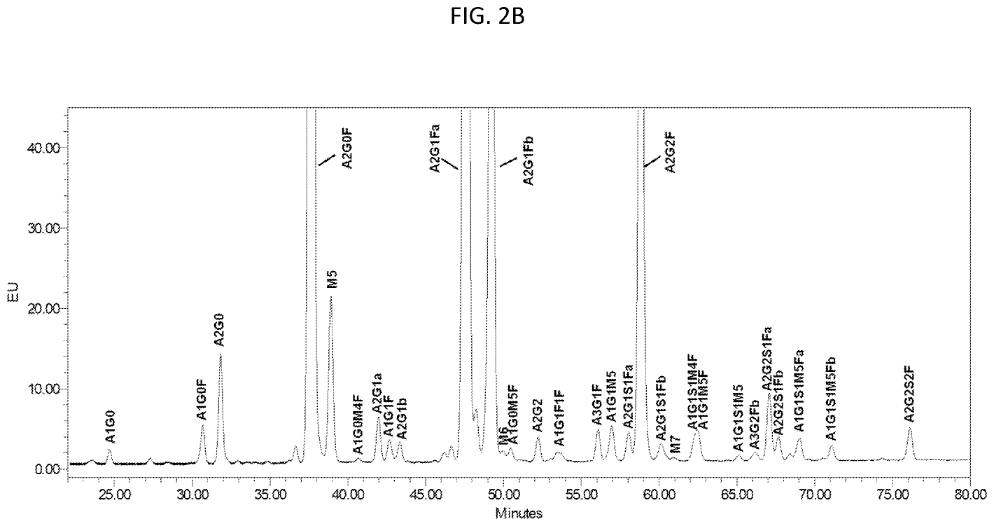

Various methods are known in the art for assessing glycans present in a glycoprotein-containing composition or for determining, detecting or measuring a glycoform profile (e.g., a glycoprofile) of a particular sample comprising glycoproteins. Suitable methods include, but are not limited to, positive ion MALDI-TOF analysis, negative ion MALDI-TOF analysis, weak anion exchange (WAX) chromatography, normal phase chromatography (NP-HPLC), exoglycosidase digestion, Bio-Gel P-4 chromatography, anion-exchange chromatography and one-dimensional n.m.r. spectroscopy, and combinations thereof. See, e.g., Mattu et al., JBC 273: 2260-2272 (1998); Field et al., Biochem J 299(Pt 1): 261-275 (1994); Yoo et al., MAbs 2(3): 320-334 (2010) Wuhrer M. et al., Journal of Chromatography B, 2005, Vol. 825, Issue 2, pages 124-133; Ruhaak L. R., Anal Bioanal Chem, 2010, Vol. 397:3457-3481 and Geoffrey, R. G. et. al. Analytical Biochemistry 1996, Vol. 240, pages 210-226. Also, Example 1 set forth herein describes a suitable method for assessing glycans present in a glycoprotein containing composition, e.g., an antibody composition. The method of Example 1 describes an assay in which glycans attached to glycosylated proteins of a composition, e.g., antibodies of an antibody composition, are enzymatically cleaved from the protein (e.g., antibody). The glycans are subsequently separated by Hydrophilic Interaction Liquid Chromatography (HILIC) and a chromatogram with several peaks is produced. Each peak of the chromatogram represents a mean distribution (amount) of a different glycan. Two views of a representative HILIC chromatogram comprising peaks for different glycans are provided in FIGS. 2A and 2B. For these purposes, % Peak Area=Peak Area/Total Peak Area x 100%, and % Total Peak Area=Sample Total Area/Total Area of the Standard x 100%. Accordingly, the level of a particular glycan (or groups of glycans) is reported as a %. For example, if an antibody composition is characterized as having a Man6 level of 30%, it is meant that 30% of all glycans cleaved from the antibodies of the composition are Man6.

The present disclosure, including the associations and equations presented herein, relates to total afucosylated glycans, high mannose glycans, and afucosylated glycans of an antibody composition. As used herein, “total afucosylated glycans” or “TAF glycans” refers to the sum amount of high mannose (HM) glycans and afucosylated glycans. As used herein, the term “high mannose glycans” or “HM glycans” encompasses glycans comprising 5, 6, 7, 8, or 9 mannose residues, abbreviated as Man5, Man6, Man7, Man8, and Man9, respectively. A level of HM glycans, in various aspects, is obtained by summing the % Man5, the % Man6, the % Man7, the % Man8, and the % Man9. As used herein, the term “afucosylated glycan” or “AF glycan” refers to glycans which lack a core fucose, e.g., an α1,6-linked fucose on the GlcNAc residue involved in the amide bond with the Asn of the N-glycosylation site. Afucosylated glycans include, but are not limited to, A1G0, A2G0, A2G1a, A2G1b, A2G2, and A1G1M5. Additional afucosylated glycans include, e.g., A1G1a, G0[H3N4], G0[H4N4], G0[H5N4], FO-N[H3N3]. See, e.g., Reusch and Tejada, Glycobiology 25(12): 1325-1334 (2015). A level of afucosylated glycans, in various aspects, is obtained by summing the % A1G0, the % A2G0, the % A2G1a, the % A2G1b, the % A2G2, the % A1G1M5, the % A1G1a, the % G0[H3N4], the % G0[H4N4], the % G0[H5N4], and the % FO-N[H3N3].

In exemplary aspects, the level of glycans (e.g., the glycan content, optionally, expressed as a %, e.g., % TAF glycans, % HM glycans, % AF glycans) is determined (e.g., measured) by any of the various methods known in the art for assessing glycans present in a glycoprotein-containing composition or for determining, detecting or measuring a glycoform profile (e.g., a glycoprofile) of a particular sample comprising glycoproteins. In exemplary instances, the level of glycans (e.g., % TAF glycans, % HM glycans, % AF glycans) of an antibody composition is determined by measuring the level of such glycans in a sample of the antibody composition though a chromatography based method, e.g., HILIC, and the level of glycans is expressed as a %, as described herein. See, e.g., Example 1. In exemplary instances, the level of glycans of an antibody composition is expressed as a % of all glycans cleaved from the antibodies of the composition. In various aspects, the % TAF glycans is determined by calculating the sum of the % high mannose glycans and the % afucosylated glycans and the % high mannose glycans and the % afucosylated glycans are determined by hydrophilic interaction chromatography, e.g., the method described in Example 1. In various aspects, the level of glycans (e.g., % TAF glycans, % HM glycans, % AF glycans) is determined (e.g., measured) by measuring the level of such glycans in a sample of the antibody composition. In exemplary instances, at least 5, at least 6, at least 7, at least 8, or at least 9 samples of an antibody composition are taken and the level of glycans (e.g., % TAF glycans, % HM glycans, % AF glycans) for each sample is determined (e.g., measured). In various aspects, the mean or average of the % TAF glycans, % HM glycans, and/or % AF glycans is determined.

In exemplary aspects, the level of glycans (e.g., % TAF glycans, % HM glycans, % AF glycans) is calculated using Equation A or Equation B, as further described herein.

ADCC

The present disclosure, including the associations and equations presented herein, relates the % total afucosylated glycans or the % high mannose glycans and % afucosylated glycans of an antibody composition to the level of ADCC activity, e.g., % ADCC, of the antibody composition.

The term “ADCC” or “antibody-dependent cell-mediated cytotoxicity” or “antibody-dependent cellular cytotoxicity” refers to the mechanism by which an effector cell of the immune system (e.g., natural killer cells (NK cells), macrophages, neutrophils, eosinophils) actively lyses a target cell, whose membrane-surface antigens have been bound by specific antibodies. ADCC is a part of the adaptive immune response and occurs when antigen-specific antibodies bind to (1) the membrane-surface antigens on a target cell through its antigen-binding regions and (2) to Fc receptors on the surface of the effector cells through its Fc region. Binding of the Fc region of the antibody to the Fc receptor causes the effector cells to release cytotoxic factors that lead to death of the target cell (e.g., through cell lysis or cellular degranulation).

Fc receptors are receptors on the surfaces of B lymphocytes, follicular dendritic cells, NK cells, macrophages, neutrophils, eosinophils, basophils, platelets and mast cells that bind to the Fc region of an antibody. Fc receptors are grouped into different classes based on the type of antibody that they bind. For example, an Fc-gamma receptor is a receptor for the Fc region of an IgG antibody, an Fc-alpha receptor is a receptor for the Fc region of an IgA antibody, and an Fc-epsilon receptor is a receptor for the Fc region of an IgE antibody.

The term “FcγR” or “Fc-gamma receptor” is a protein belonging to the IgG superfamily involved in inducing phagocytosis of opsonized cells or microbes. See, e.g., Fridman W H. Fc receptors and immunoglobulin binding factors. FASEB Journal. 5 (12): 2684-90 (1991). Members of the Fc-gamma receptor family include: FcγRI (CD64), FcγRIIA (CD32), FcγRIIB (CD32), FcγRIIIA (CD16a), and FcγRIIIB (CD16b). The sequences of FcγRI, FcγRIIA, FcγRIIB, FcγRIIIA, and FcγRIIIB can be found in many sequence databases, for example, at the Uniprot database (www.uniprot.org) under accession numbers P12314 (FCGR1_HUMAN), P12318 (FCG2A_HUMAN), P31994 (FCG2B_HUMAN), P08637 (FCG3A_HUMAN), and P08637 (FCG3A_HUMAN), respectively.

The term “ADCC activity” or “ADCC level” or “ADCC activity level” refers to the extent to which ADCC is activated or stimulated. Methods of measuring or determining the ADCC level of an antibody composition, including commercially available assays and kits for measuring or determining the ADCC level, are well-known in the art, as described, Yamashita et al., Scientific Reports 6: article number 19772 (2016), doi:10.1038/srep19772); Kantakamalakul et al., “A novel EGFP-CEM-NKr flow cytometric method for measuring antibody dependent cell mediated-cytotoxicity (ADCC) activity in HIV-1 infected individuals”, J Immunol Methods 315 (Issues 1-2): 1-10; (2006); Gomez-Roman et al., “A simplified method for the rapid fluorometric assessment of antibody-dependent cell-mediated cytotoxicity”, J Immunol Methods 308 (Issues 1-2): 53-67 (2006); Schnueriger et al., Development of a quantitative, cell-line based assay to measure ADCC activity mediated by therapeutic antibodies”, Molec Immunology 38 (Issues 12-13): 1512-1517 (2011); and Mata et al., “Effects of cryopreservation on effector cells for antibody dependent cell-mediated cytotoxicity (ADCC) and natural killer (NK) cell activity in ⁵¹Cr-release and CD107a assays”, J Immunol Methods 406: 1-9 (2014); all herein incorporated by reference for all purposes. The term “ADCC Assay” or “FcγR reporter gene assay” refers to an assay, kit or method useful to determine the ADCC activity of an antibody. Exemplary methods of measuring or determining the ADCC activity of an antibody in the methods described herein include the ADCC assay described in the Example 2 or the ADCC Reporter Assay commercially available from Promega (Catalog No. G7010 and G7018). In some embodiments, ADCC activity is measured or determined using a calcein release assay containing one or more of the following: a FcγRIIa (158V)-expressing NK92(M1) cells as effector cells and HCC2218 cells or WIL2-S cells as target cells labeled with calcein-AM.

In exemplary aspects, the level of ADCC of an antibody composition is determined by a quantitative cell-based assay which measures the ability of the antibodies of the antibody composition to mediate cell cytotoxicity in a dose-dependent manner in cells expressing the antigen of the antibodies and engaging Fc-gammaRIIIA receptors on effector cells through the Fc domain of the antibodies. In various embodiments, the method comprises the use of target cells harboring detectable labels that are released when the target cells are lysed by the effector cells. The amount of detectable label released from the target cells is a measure of the ADCC activity of the antibody composition. The amount of detectable label released from the target cells in some aspects is compared to a baseline. Also, the ADCC level may be reported as a % ADCC relative to a control % ADCC. In various aspects, the % ADCC is a relative % ADCC, which optionally, is relative to a control % ADCC. In various aspects, the control % ADCC is the % ADCC of a reference antibody. In various aspects, the reference antibody is rituximab. In exemplary instances, the control % ADCC is within a range of about 60% to about 130%. Optionally, the % ADCC is determined by the assay described in Example 2.

The present disclosure relates the TAF glycan content, HM glycan content, and/or AF glycan content of an antibody composition to the ADCC activity level of the antibody composition. As demonstrated herein, the % TAF glycans, % HM glycans, and/or % AF glycans of an antibody composition are related to the % ADCC activity of the antibody composition. In various aspects, based on a first model which correlates TAF glycan content to ADCC activity level, either (a) the ADCC activity level is calculated based on the TAF glycan content (e.g., the TAF glycan content is measured) or (b) the TAF glycan content is calculated based on the ADCC activity level (e.g., the ADCC activity level is measured). In various instances, a target ADCC activity level or target range of ADCC activity levels is known, given the particular antibody of the antibody composition being produced. For example, the antibody may be a biosimilar of a reference antibody and the target ADCC activity level or a range thereof is known for the reference antibody. In exemplary aspects, the target TAF glycan content or a target range of TAF glycan content may be calculated based on the first model. In various instances, the first model is a linear regression model. In various instances, the first model is a simplified version of a linear regression model without a y-intercept. In various aspects, the first model which correlates ADCC and TAF glycan content is statistically significant as demonstrated by its low p-value. In various aspects, the p-value is less than 0.0001.

In exemplary aspects, the first model correlates ADCC activity level of the antibody composition as about 13.5%±0.5% for every 1% TAF glycan content present in the antibody composition, optionally, wherein the antibody of the antibody composition binds to an antigen comprising only one antibody binding site. In various aspects, the first model correlates ADCC activity level of the antibody composition as about 24.74%±0.625% for every 1% TAF glycan content present in the antibody composition, optionally, wherein the antibody of the antibody composition binds to an antigen comprising only two antibody binding sites. In exemplary aspects, the first model correlates ADCC activity level of the antibody composition as about 12%±1.5%*Q for every 1% TAF glycan content present in the antibody composition, wherein Q is the number of antibody binding sites present on the antigen. In exemplary instances, Q is 1 and optionally the antibody is infliximab or a biosimilar thereof. Optionally, Q is 2 and optionally the antibody is rituximab or a biosimilar thereof.

In various aspects, the target range of ADCC activity levels is known, pre-selected or pre-determined and the first model allows for the calculation of a target range for TAF glycan content based on this target range of ADCC activity levels. In exemplary instances, the target range of TAF glycan content is m to n, wherein m is [ADCCₘᵢₙ/12Q], wherein ADCCₘᵢₙ is the minimum of the target range of ADCC activity level for a reference antibody, and n is [ADCCₘₐₓ]/12Q], wherein ADCCₘₐₓ is the maximum of the target range of ADCC activity level for the reference antibody. In various instances, Q is 2. In various instances, the ADCC activity level predicted by the first model is ^{˜}24*% TAF. In various instances, the target range of TAF glycan content is m to n wherein m is [ADCCₘᵢₙ/24] and n is [ADCCₘₐₓ]/24]. In various instances, Q is 1. In various aspects, the ADCC activity level predicted by the first model is ^{˜}12*% TAF. In various instances, the target range of TAF glycan content is m to n wherein m is [ADCCₘᵢₙ/12] and n is [ADCCₘₐₓ]/12]. In various aspects, the target range for TAF glycan content is m° to n°, wherein m° is [[ADCCₘᵢₙ−y]/x], wherein ADCCₘᵢₙ is the minimum of the target range of ADCC activity level, and n° is [[ADCCₘₐₓ−y]/x], wherein ADCCₘₐₓ is the maximum of the target range of ADCC activity level. Optionally, x is about 20.4 to about 27.7 and y is about −11.4 to about 16.7. Alternatively, x is about 9.7 to about 15.2 and y is about −15.6 to about 34.2. In various aspects, the target range for TAF glycan content is m′ to n′, wherein m′ is [ADCCₘᵢₙ/x′], wherein ADCCₘᵢₙ is the minimum of the target range of ADCC activity level, and n′ is [ADCCₘₐₓ]/x′], wherein ADCCₘₐₓ is the maximum of the target range of ADCC activity level. Optionally, x′ is about 24.1 to about 25.4. Alternatively, x′ is about 13.0 to about 13.95. In various instances, the ADCC activity level of the antibody composition is about 13.5%±0.5% for every 1% TAF present in the antibody composition, optionally, wherein the antibody of the antibody composition binds to an antigen comprising only one antibody binding site. In various aspects, the ADCC activity level of the antibody composition is about 24.74%±0.625% for every 1% TAF present in the antibody composition, optionally, wherein the antibody of the antibody composition binds to an antigen comprising only two antibody binding sites. In exemplary aspects, the ADCC activity level of the antibody composition is about 12%±1.5%*Q for every 1% TAF present in the antibody composition, Q is the number of antibody binding sites present on the antigen. In exemplary instances, the reference antibody is infliximab. In exemplary aspects, the reference antibody is rituximab.

The ADCC activity or % ADCC may be calculated using an equation which relates the % TAF glycans, % HM glycans, and/or % AF glycans to the % ADCC activity of a given antibody composition. In various aspects, the equation relates the % TAF glycans to the % ADCC. In exemplary aspects, the equation is Equation A:

Y=2.6+24.1*X [Equation A],

wherein Y is the % ADCC and X is the % TAF glycans.

In various instances, the equation relates the % HM glycans and the % AF glycans to the % ADCC of the antibody composition. In exemplary aspects, the equation is Equation B:

Y=(0.24+27*HM+22.1*AF) [Equation B],

wherein Y is the % ADCC, HM is the % high mannose glycans, and AF is the % afucosylated glycans.

In exemplary aspects, the method comprises determining (e.g., measuring) the % TAF glycans, and by using the determined (e.g., measured) % TAF glycans, the % ADCC may be calculated using Equation A. Accordingly, in exemplary instances, the method comprises calculating the % ADCC of the antibody composition based on the determined (e.g., measured) % TAF glycans using Equation A. In various aspects, the % ADCC calculated in such manner is useful for not needing to experimentally determine (e.g., measure the % ADCC) of an antibody composition.

In exemplary aspects, the method comprises determining (e.g., measuring) the % HM glycans and the % AF glycans, and by using the determined (e.g., measured) % HM glycans and % AF glycans, the % ADCC may be calculated using Equation B. Accordingly, in exemplary instances, the method comprises calculating the % ADCC of the antibody composition based on the determined (e.g., measured) % HM glycans and % AF glycans using Equation B. In various aspects, the % ADCC calculated in such manner is useful for not needing to experimentally determine (e.g., measure the % ADCC) of an antibody composition.

In various aspects, the presently disclosed equations relating % ADCC and % TAF glycans, % HM glycans, and/or % AF glycans may be re-expressed so that, for example, a % TAF glycans may be determined using the equation. For instance, Equation A may be re-expressed as follows:

X=(Y−2.6)/24.1

wherein Y is the % ADCC and X is the % TAF glycans.

Alternatively, Equation B may be re-expressed as follows:

(Y−0.24)=27*HM+22.1*AF; or

[(Y−0.24)−22.1*AF]/27=HM; or

[(Y−0.24)−27*HM]/22.1=AF,

wherein Y is the % ADCC, HM is the % high mannose glycans, and AF is the % afucosylated glycans.

In exemplary instances, the % ADCC is determined (e.g., measured) and by using the determined % ADCC in the re-expression of Equation A, the % TAF related to the determined % ADCC may be calculated. The % TAF calculated using Equation A and the determined % ADCC is useful for identifying a target % TAF in order to achieve a particular % ADCC. Also, in exemplary aspects, the % ADCC is determined (e.g., measured) and by using the determined % ADCC in the re-expression of Equation B, the % HM glycans or the % AF glycans may be calculated.

In various aspects, the % ADCC is a target % ADCC and the method identifies a target % TAF glycans using the target ADCC level. The method in various aspects, comprises maintaining glycosylation-competent cells in a cell culture to produce an antibody composition with the target % TAF level, as calculated using Equation A. Once the antibody composition achieves the target % TAF level, the method may comprise carrying out one or more downstream processing steps with the antibody composition. In various aspects, the method optionally comprises confirming the actual % TAF of the antibody composition.

In various aspects, the methods comprise selecting the antibody composition for one or more downstream processing steps when Y as calculated using the determined % TAF glycans with Equation A or the % HM glycans and the % AF glycans with Equation B is within a target ADCC range.

Methods of Determining and/or Monitoring Product Quality

Based on these correlations, product quality of an antibody composition may be determined and/or monitored. Accordingly, the present disclosure provides methods of determining product quality of an antibody composition, wherein the ADCC activity level of the antibody composition is a criterion upon which product quality of the antibody composition is based. In exemplary embodiments, the method comprises (i) determining the total afucosylated (TAF) glycan content of a sample of an antibody composition; and (ii) determining the product quality as acceptable and/or achieving the ADCC activity level criterion when the TAF glycan content determined in (i) is within a target range. In exemplary aspects, the target range of TAF glycan content is based on (1) a target range of ADCC activity levels for a reference antibody and (2) a first model which correlates ADCC activity level of the antibody composition to TAF glycan content of the antibody composition. In exemplary aspects, the ADCC predicted by the first model is about 95% to about 105% of the ADCC predicted by a second model, wherein the second model correlates the ADCC activity level of the antibody composition to the HM glycan content of the antibody composition and the AF glycan content of the antibody composition.

Advantageously, the ADCC predicted by the first model is statistically significantly similar to the ADCC predicted by the second model. For example, the ADCC activity level predicted by the first model is about 95% to about 105% of the ADCC activity level predicted by the second model. Optionally, the ADCC activity level predicted by the first model is about 95%, about 96%, about 97%, about 98%, about 99%, about 100%, about 101%, about 102%, about 103%, about 104%, or about 105% of the ADCC activity level predicted by the second model. The ADCC activity level predicted by the first model is, in various instances, about 100% of the ADCC predicted by the second model. In certain aspects, there is a one-to-one correspondence between the ADCC predicted by the first model and the ADCC predicted by the second model. In various instances, the first model and/or the second model is/are statistically significant. For instance, the p-value of the first model is less than 0.0001 and/or the p-value of the second model is less than 0.0001. Optionally, each of the first model and the second model has a p-value which is less than 0.0001.

In exemplary aspects, the ADCC activity level predicted by the first model is ^{˜}12Q*% TAF, wherein Q is the number of antibody binding sites on the antigen to which the antibody binds and % TAF is the TAF glycan content of the antibody composition. In exemplary instances, the target range of TAF glycan content is m to n, wherein m is [ADCCₘᵢₙ/12Q], wherein ADCCₘᵢₙ is the minimum of the target range of ADCC activity level for a reference antibody, and n is [ADCCₘₐₓ]/12Q], wherein ADCCₘₐₓ is the maximum of the target range of ADCC activity level for the reference antibody. In various instances, Q is 2. In various instances, the ADCC activity level predicted by the first model is ^{˜}24*% TAF. In various instances, the target range of TAF glycan content is m to n wherein m is [ADCCₘᵢₙ/24] and n is [ADCCₘₐₓ]/24]. In various instances, the ADCC activity level predicted by the second model is ^{˜}27*% HM+^{˜}22*% AF, wherein % AF is the AF glycan content of the antibody composition and % HM is the HM glycan content of the antibody composition. In various instances, Q is 1. In various aspects, the ADCC activity level predicted by the first model is ^{˜}12*% TAF. In various instances, the target range of TAF glycan content is m to n wherein m is [ADCCₘᵢₙ/12] and n is [ADCCₘₐₓ]/12]. In various instances, the ADCC activity level predicted by the second model is ^{˜}14.8*% HM+^{˜}12.8*% AF. Suitable alternative first models and second models are described herein. In exemplary instances, the first model is any of one of the models (e.g., equations) described herein which correlate ADCC and TAF glycan content, including but not limited to, Equations 1, 3, 5, and 7 and Equation A. In exemplary instances, the second model is any of one of the models (e.g., equations) described herein which correlate ADCC and HM glycan content and AF glycan content, including but not limited to, Equations 2, 4, 6, and 8 and Equation B. For example, in various aspects, the target range for TAF glycan content is m° to n°, wherein m° is defined as [[ADCCₘᵢₙ−y]/x], wherein ADCCₘᵢₙ is the minimum of the target range of ADCC activity level, and n° is defined as [[ADCCₘₐₓ−y]/x], wherein ADCCₘₐₓ is the maximum of the target range of ADCC activity level. Optionally, x is about 20.4 to about 27.7 and y is about −11.4 to about 16.7. Alternatively, x is about 9.7 to about 15.2 and y is about −15.6 to about 34.2. In various aspects, the target range for TAF glycan content is m′ to n′, wherein m′ is [ADCCₘᵢₙ/x′], wherein ADCCₘᵢₙ is the minimum of the target range of ADCC activity level, and n′ is [ADCCₘₐₓ]/x′], wherein ADCCₘₐₓ is the maximum of the target range of ADCC activity level. Optionally, x′ is about 24.1 to about 25.4. Alternatively, x′ is about 13.0 to about 13.95. In various instances, the ADCC activity level of the antibody composition is about 13.5%±0.5% for every 1% TAF present in the antibody composition, optionally, wherein the antibody of the antibody composition binds to an antigen comprising only one antibody binding site. In various aspects, the ADCC activity level of the antibody composition is about 24.74%±0.625% for every 1% TAF present in the antibody composition, optionally, wherein the antibody of the antibody composition binds to an antigen comprising only two antibody binding sites. In exemplary aspects, the ADCC activity level of the antibody composition is about 12%±1.5%*Q for every 1% TAF present in the antibody composition, Q is the number of antibody binding sites present on the antigen.

In exemplary aspects, the antibody binds to an antigen which comprises only one antibody binding site. In exemplary instances, the reference antibody is infliximab. In exemplary aspects, the antibody binds to an antigen which comprises only two antibody binding sites. In exemplary aspects, the reference antibody is rituximab.

In exemplary aspects, the method is a quality control (QC) assay. In exemplary aspects, the method is an in-process QC assay. In various aspects, the sample is a sample of in-process material. In various instances, the TAF glycan content is determined pre-harvest or post-harvest. In exemplary instances, the TAF glycan content is determined after a chromatography step. Optionally, the chromatography step comprises a capture chromatography, intermediate chromatography, and/or polish chromatography. In some aspects, the TAF glycan content is determined after a virus inactivation and neutralization, virus filtration, or a buffer exchange. The method in various instances is a lot release assay. The sample in some aspects is a sample of a manufacturing lot.

In various aspects, the method further comprises selecting the antibody composition for downstream processing, when the TAF glycan content determined in (i) is within a target range. When the TAF glycan content determined in (i) is not within the target range, one or more conditions of the cell culture are modified to obtain a modified cell culture, in various aspects. The method, in some aspects, further comprises determining the TAF glycan content of a sample of the antibody composition obtained after one or more conditions of the cell culture are modified, e.g., determining the TAF glycan content of a sample of the antibody composition of the modified cell culture. In various aspects, when the TAF glycan content determined in (i) is not within the target range, the method further comprises (iii) modifying one or more conditions of the cell culture to obtain a modified cell culture and (iv) determining the TAF glycan content of a sample of the antibody composition obtained from the modified cell culture. In exemplary aspects, when the TAF glycan content determined in (i) is not within the target range, the method further comprises (iii) and (iv) until the TAF glycan content determined in (iv) is within the target range. In exemplary instances, an assay which directly measures ADCC activity of the antibody composition is carried out on the antibody composition only when the TAF glycan content determined in (i) is not within the target range, e.g., outside the target range. Assays which directly measure ADCC activity include for example a cell-based assay that measures the release of a detectable reagent upon lysis of antigen-expressing cells comprising the detectable agent by effector cells that are bound to antibody binding both antigen-expressing and effector cells. In exemplary instances, an assay which directly measures ADCC activity of the antibody composition is not carried out on the antibody composition. In various aspects, determining the TAF glycan content is the only step required to determine the product quality with regard to the ADCC activity level criterion. Without being bound to theory, the statistically significant correlations of the first model and the second model allow for TAF glycan content to indicate ADCC activity level such that assays that directly measure ADCC activity level are not needed. Accordingly, direct measurement of the ADCC activity level of the antibody composition is not needed and thus not carried out in various aspects of the presently disclosed methods.

In various aspects, the method determines the product quality in terms of the ADCC activity level criterion. In various aspects, the ADCC activity level criterion is one of the acceptance criteria for the antibody composition. The presently disclosed methods in various aspects are purposed to assure that batches of drug products meet each appropriate specification and appropriate statistical quality control criteria as a condition for their approval and release, pursuant to 21 CFR 211.165. In various aspects, the presently disclosed methods of determining product quality meet the statistical quality control criteria which includes appropriate acceptance levels and/or appropriate rejection levels. Terminology, including, but not limited to “acceptance criteria”, “lot” and “in-process” accord with their meaning as defined in 21 Code of Federal Regulations (CFR) Section 210.3.

The present disclosure also provides methods of monitoring product quality of an antibody composition, wherein the ADCC activity level of the antibody composition is a criterion upon which product quality of the antibody composition is based. In exemplary embodiments, the method comprises determining product quality of an antibody composition in accordance with a method of the present disclosures, with a first sample obtained at a first timepoint and with a second sample taken at a second timepoint which is different from the first timepoint. In various instances, each of the first sample and second sample is a sample of in-process material. In various aspects, the first sample is a sample of in-process material and the second sample is a sample of a manufacturing lot. Optionally, the first sample is a sample obtained before one or more conditions of the cell culture are modified and the second sample is a sample obtained after the one or more conditions of the cell culture are modified. In exemplary instances, the TAF glycan content is determined for each of the first sample and second sample. Additional samples may be obtained for purposes of determining product quality of the antibody composition and for determining TAF glycan content. Product quality of the antibody composition depends on whether the TAF glycan content is within a target range. In exemplary aspects, the target range of TAF glycan content is based on (1) a target range of ADCC activity levels for a reference antibody and (2) a first model which correlates ADCC activity level of the antibody composition to TAF glycan content of the antibody composition. In exemplary aspects, the ADCC predicted by the first model is about 95% to about 105% of the ADCC predicted by a second model, wherein the second model correlates the ADCC activity level of the antibody composition to the HM glycan content of the antibody composition and the AF glycan content of the antibody composition.

Methods of Producing Antibody Compositions

The present disclosure provides methods of producing an antibody composition. In exemplary embodiments, the method comprises determining product quality of the antibody composition wherein product quality of the antibody composition is determined in accordance with a method of the present disclosures. Optionally, the method comprises determining the TAF glycan content of a sample of an antibody composition and the sample is a sample of in-process material. In various instances, the method comprises determining the product quality of the antibody composition as acceptable and/or achieving the ADCC activity level criterion when the TAF glycan content determined in (i) is within a target range, as defined herein. In exemplary aspects, the target range of TAF glycan content is based on (1) a target range of ADCC activity levels for a reference antibody and (2) a first model which correlates ADCC activity level of the antibody composition to TAF glycan content of the antibody composition. In exemplary aspects, the ADCC predicted by the first model is about 95% to about 105% of the ADCC predicted by a second model, wherein the second model correlates the ADCC activity level of the antibody composition to the HM glycan content of the antibody composition and the AF glycan content of the antibody composition. In various aspects, when the TAF glycan content determined in (i) is not within the target range, the method further comprises (iii) modifying one or more conditions of the cell culture to obtain a modified cell culture and (iv) determining the TAF glycan content of a sample of the antibody composition obtained from the modified cell culture, optionally, repeating steps (iii) and (iv) until the TAF glycan content is within the target range. in various instances, the sample is a sample of a cell culture comprising cells expressing an antibody of the antibody composition. In various instances, one or more conditions of the cell culture are modified to modify the TAF glycan content. In various aspects, the TAF glycan content of the antibody composition is achieved by modifying the AF glycan content. In exemplary aspects, one or more conditions of the cell culture are modified to modify the AF glycan content of the antibody composition. In exemplary aspects, the one or more conditions primarily modify the AF glycan content. In various instances, the one or more conditions modify the AF glycan content and does not modify the HM glycan content. In exemplary aspects, the method comprises the TAF glycan content of the antibody composition is achieved by modifying the HM glycan content. Optionally, one or more conditions of the cell culture are modified to modify the HM glycan content of the antibody composition. In some instances, the one or more conditions primarily modify the HM glycan content. In some aspects, the one or more conditions modify the HM glycan content and does not modify the AF glycan content. In various instances, the method comprises repeating the modifying of the afucosylated (AF) glycan content and/or repeating the modifying of the high mannose (HM) glycan, until the TAF glycan content is within a target range.

In exemplary embodiments, the method of producing an antibody composition comprises (i) determining the total afucosylated (TAF) glycan content of a sample of an antibody composition; and (ii) selecting the antibody composition for downstream processing based on the TAF glycan content determined in (i). In various aspects, the sample is taken from a cell culture comprising cells expressing an antibody of the antibody composition. In various instances, the method further comprises modifying the TAF glycan content of the antibody composition and determining the modified TAF glycan content. Optionally, one or more conditions of the cell culture are modified in order to modify the TAF glycan content. In exemplary aspects, the method comprises repeating the modifying until the TAF glycan content is within a target range. In exemplary instances, the target range is based on a target range of ADCC activity level for the antibody. Without being bound to theory, the TAF glycan content correlates with the ADCC activity level of the antibody composition such that the ADCC activity level of an antibody composition may predicted based on the TAF glycan content of the antibody composition. The ADCC activity level of the antibody composition may be a criteria worth considering when deciding whether the antibody composition should be selected for downstream processing. Therefore, in various aspects, the method comprises (i) determining the TAF glycan content of a sample of an antibody composition; (ii) determining the ADCC activity level of the antibody composition based on the TAF glycan content determined in (i), and, optionally, (iii) selecting the antibody composition for downstream processing when the ADCC level of the antibody composition determined in (ii) is within a target range of ADCC activity level. In various aspects, the target range of ADCC activity level is known for the antibody of the antibody composition. The antibody of the antibody composition, in various aspects, is a biosimilar of a reference antibody. In various instances, a target range of TAF glycan content is based or determined (e.g., calculated) based on the target range of ADCC activity level which is known. Accordingly, in exemplary aspects, the method comprises (i) determining the TAF glycan content of a sample of an antibody composition; and (ii) selecting the antibody composition for downstream processing when the TAF glycan content determined in (i) is within a target range. When the method further comprises modifying the TAF glycan content of the antibody composition, the method in various instances comprises modifying the afucosylated (AF) glycan content to modify the TAF glycan content. Optionally, one or more conditions of the cell culture are modified to modify the AF glycan content of the antibody composition, which, in turn, modifies the TAF glycan content. Alternatively or additionally, when the method further comprises modifying the TAF glycan content of the antibody composition, the method in various instances comprises modifying the high mannose (HM) glycan content to modify the TAF glycan content. Optionally, one or more conditions of the cell culture are modified to modify the HF glycan content of the antibody composition, which, in turn, modifies the TAF glycan content. In exemplary aspects, the one or more conditions primarily modify the AF glycan content. In exemplary instances, the one or more conditions primarily modify the HM glycan content. In exemplary aspects, the one or more conditions modify the AF glycan content and not the HM glycan content. In exemplary instances, the one or more conditions modify the HM glycan content and not the AF glycan content. The method optionally comprises repeating the modifying of the afucosylated (AF) glycan content and/or repeating the modifying of the high mannose (HM) glycan, until the TAF glycan content is within a target range. In exemplary aspects, the antibody of the antibody composition is an IgG, optionally, an IgG₁. In various aspects, the target range for TAF glycan content is m to n, wherein m is [[ADCCₘᵢₙ−y]/x], wherein ADCCₘᵢₙ is the minimum of the target range of ADCC activity level, and n is [[ADCCₘₐₓ−y]/x], wherein ADCCₘₐₓ is the maximum of the target range of ADCC activity level. Optionally, x is about 20.4 to about 27.7 and y is about −11.4 to about 16.7. Alternatively, x is about 9.7 to about 15.2 and y is about −15.6 to about 34.2. In various aspects, the target range for TAF glycan content is m′ to n′, wherein m′ is [ADCCₘᵢₙ/x′], wherein ADCCₘᵢₙ is the minimum of the target range of ADCC activity level, and n′ is [ADCCₘₐₓ]/x′], wherein ADCCₘₐₓ is the maximum of the target range of ADCC activity level. Optionally, x′ is about 24.1 to about 25.4. Alternatively, x′ is about 13.0 to about 13.95. In various instances, the ADCC activity level of the antibody composition is about 13.5%±0.5% for every 1% TAF present in the antibody composition, optionally, wherein the antibody of the antibody composition binds to an antigen comprising only one antibody binding site. In various aspects, the ADCC activity level of the antibody composition is about 24.74%±0.625% for every 1% TAF present in the antibody composition, optionally, wherein the antibody of the antibody composition binds to an antigen comprising only two antibody binding sites. In exemplary aspects, the ADCC activity level of the antibody composition is about 12%±1.5%*Q for every 1% TAF present in the antibody composition, Q is the number of antibody binding sites present on the antigen. In exemplary instances, Q is 1 and optionally the antibody is infliximab or a biosimilar thereof. Optionally, Q is 2 and optionally the antibody is rituximab or a biosimilar thereof.

The presently disclosed methods of producing an antibody composition comprises modifying total afucosylated (TAF) glycan content of an antibody composition produced by cells of a cell culture. In various instances, one or more conditions of the cell culture are modified to modify the TAF glycan content. In various aspects, the method comprises determining the modified TAF glycan content. Optionally, the modifying is repeated until the determined TAF glycan content is in a target range of TAF. Without being bound to a particular theory, the TAF glycan content may be modified by changing the afucosylated (AF) glycan content or the high mannose (HM) content, or a combination thereof, since each impacts the TAF glycan content. Accordingly, the methods advantageously allow for multiple ways to achieve the target range of TAF glycan content. For example, one or more conditions of the cell culture are modified to modify the AF glycan content in order to modify the TAF glycan content. Alternatively, one or more conditions of the cell culture are modified to modify the HM glycan content in order to modify the TAF glycan content. In various instances, one or more conditions of the cell culture are modified to modify the AF glycan content and the HM glycan content in order to modify the TAF glycan content. Therefore, the present disclosure further provides methods of modifying total afucosylated (TAF) glycan content of an antibody composition produced by cells of a cell culture. In exemplary embodiments, the method comprises modifying the AF glycan content. In exemplary embodiments, the method comprises modifying the HM glycan content. In various aspects, the method comprises (i) determining the afucosylated (AF) glycan content and the high mannose (HM) glycan content of a sample of an antibody composition; (ii) determining a target range of AF glycan content based on a target range of ADCC activity level of an antibody of the antibody composition, assuming the HM glycan content is constant; and (iii) selecting the antibody composition for downstream processing when the AF glycan content is in the target range of AF glycan content. In various instances, the method comprises (i) determining the afucosylated (AF) glycan content and the high mannose (HM) glycan content of a sample of an antibody composition; (ii) determining a target range of HM glycan content based on a target range of ADCC activity level of an antibody of the antibody composition, assuming the AF glycan content is constant; and (iii) selecting the antibody composition for downstream processing when the HM glycan content is in the target range of AF glycan content. In various instances, the method comprises (i) determining the AF glycan content and the HM glycan content of a sample of the antibody composition and (ii) determining a target range of AF glycan content based on the HM glycan content determined in (i), and (iii) modifying the AF glycan content until it is within the target range of AF glycan content, wherein the HM glycan content is unmodified. Alternatively, the method comprises (i) determining the AF glycan content and the HM glycan content of a sample of the antibody composition and (ii) determining a target range of HM glycan content based on the AF glycan content determined in (i), and (iii) modifying the HM glycan content until it is within the target range of HM glycan content, wherein the AF glycan content is unmodified. In exemplary aspects, the model which correlates ADCC activity level of the antibody composition to the TAF glycan content of the antibody composition predicts essentially the same ADCC activity level predicted by the model which correlates ADCC to HM and AF glycan content. Suitable methods of modifying the AF glycan content and/or HM glycan content are known in the art. For instance, International Patent Publication No. WO 2019/191150 teaches methods of modifying the level of afucosylated glycans of an antibody composition and methods of modifying the level of high mannose glycans of an antibody composition. In such methods, one or more conditions of the cell culture, e.g., pH, fucose concentration, glucose concentration, are modified to achieve the desired level of AF glycan and/or HM glycan. Additionally, each of International Patent Publication Nos. WO 2013/114164, WO 2016/089919, WO 2013/114245, WO 2015/128793, and WO 2013/114167, U.S. Patent Application Publication No. US2014/0356910, and Konno et al., Cytotech 64: 249-265 (2012) teaches methods for obtaining increased defucosylated glycans.

In exemplary embodiments, the method of producing an antibody composition comprises (i) determining the % total afucosylated (TAF) glycans of an antibody composition; (ii) calculating a % antibody dependent cellular cytotoxicity (ADCC) of the antibody composition based on the % TAF using Equation A:

Y=2.6+24.1*X [Equation A],
wherein Y is the % ADCC and X is the % TAF glycans determined in step (i), and
   (iii) selecting the antibody composition for one or more downstream processing steps when Y is within a target % ADCC range.

In exemplary embodiments, the method of producing an antibody composition comprises (i) determining the % high mannose glycans and the % afucosylated glycans of an antibody composition, (ii) calculating a % antibody dependent cellular cytotoxicity (ADCC) of the antibody composition based on the % high mannose glycans and the % afucosylated glycans using Equation B:

Y=(0.24+27*HM+22.1*AF) [Equation B],
wherein Y is the % ADCC, HM is the % high mannose glycans determined in step (i), and AF is the % afucosylated glycans determined in step (i), and
   (iii) selecting the antibody composition for one or more downstream processing steps when Y is within a target % ADCC range.

In exemplary embodiments, the method of producing an antibody composition with a target % ADCC and the method comprises (i) calculating a target % total afucosylated (TAF) glycans for the target % ADCC using Equation A:

Y=2.6+24.1*X [Equation A],
wherein Y is the target % ADCC and X is the target % TAF glycans; and
   (ii) maintaining glycosylation-competent cells in a cell culture to produce an antibody composition with the target % TAF glycans, X.

In exemplary embodiments, the method of producing an antibody composition with a target % ADCC and the method comprises (i) calculating a target % afucosylated glycans and a target % high mannose glycans for the target % ADCC using Equation B

Y=(0.24+27*HM+22.1*AF) [Equation B],

wherein Y is the % ADCC, HM is the % high mannose glycans, and AF is the % afucosylated glycans, and (iii) maintaining glycosylation-competent cells in a cell culture to produce an antibody composition with the target % high mannose glycans and the target % afucosylated glycans.

In exemplary aspects, the target % ADCC is within a target % ADCC range. Optionally, the target % ADCC range is greater than or about 40 and less than or about 170 or about 175. For example, the target % ADCC range is about 40 to about 175, about 50 to about 175, about 60 to about 175, about 70 to about 175, about 80 to about 175, about 90 to about 175, about 100 to about 175, about 110 to about 175, about 120 to about 175, about 130 to about 175, about 140 to about 175, about 150 to about 175, about 160 to about 175, or about 170 to about 175, or about 40 to about 170, about 40 to about 160, about 40 to about 150, about 40 to about 140, about 40 to about 130, about 40 to about 120, about 40 to about 110, about 40 to about 100, about 40 to about 90, about 40 to about 80, about 40 to about 70, about 40 to about 60, or about 40 to about 50. In various aspects, the target % ADCC range is greater than or about 44 and less than or about 165 (e.g., about 45 to about 165, about 50 to about 165, about 60 to about 165, about 100 to about 165, about 45 to about 100, about 45 to about 60, about 100 to about 150, about 100 to about 125, about 125 to about 150). The target % ADCC range is in exemplary aspects is greater than or about 60 and less than or about 130.

In exemplary instances, the target % ADCC range depends on Y of Equation A or Equation B. For instance, in some aspects, the target % ADCC range is Y±20, optionally, Y±17 or Y±18. In some aspects, the target % ADCC range is Y±17 for Equation A and Y±18 for Equation B.

The target % ADCC range may be any one of those described for antibody compositions. See, e.g., Compositions.

In exemplary embodiments, the method of producing an antibody composition with a % ADCC, Y, which is optionally greater than or about 40 and less than or about 170, comprises (i) determining the % total afucosylated (TAF) glycans, X, of the antibody composition, and (ii) selecting the antibody composition for one or more downstream processing steps, when X is equivalent to (Y−2.6)/24.1. In various aspects, X is greater than or about 1.55 and less than or about 6.95, optionally, about 1.6 to about 6.9, or about 1.6 to about 6.5, about 1.6 to about 6.0, about 1.6 to about 5.5, about 1.6 to about 5.0, about 1.6 to about 4.5, about 1.6 to about 4.0, about 1.6 to about 3.5, about 1.6 to about 3.0, about 1.6 to about 2.5, about 1.6 to about 2.0, about 2.0 to about 6.95, about 2.5 to about 6.95, about 3.0 to about 6.95, about 3.5 to about 6.95, about 4.0 to about 6.95, about 4.5 to about 6.95, about 5.0 to about 6.95, about 5.5 to about 6.95, about 6.0 to about 6.95, or about 6.5 to about 6.95. In various aspects, Y is greater than or about 44 and less than or about 165, and optionally, wherein X is about 1.72 to about 6.74.

In exemplary embodiments, the method is a method of producing an antibody composition with a % ADCC, Y, said method comprising (i) determining the % total afucosylated (TAF) glycans, X, of the antibody composition, and (ii) selecting the antibody composition for one or more downstream processing steps, when the X is equivalent to (Y−2.6)/24.1, optionally, wherein X is greater than or about X−0.4 and less than or about X+0.4, and wherein the % ADCC is greater than about Y−17 and less than or about Y+17. In various instances, the X is X±0.3, X±0.2, X±0.1 and/or Y is Y±16, Y±15, Y±12, Y±9, Y±6, Y±3, Y±2, or Y±1.

In exemplary embodiments, the method is a method of producing an antibody composition with a % ADCC, said method comprising (i) determining the % afucosylated glycans and the % high mannose glycans of the antibody composition, and (ii) selecting the antibody composition for one or more downstream processing steps, when AF and HM are related to Y according to Equation B

Y=(0.24+27*HM+22.1*AF) [Equation B],

wherein Y is the % ADCC, HM is the % high mannose glycans determined in step (i), and AF is the afucosylated glycans determined in step (i).

In exemplary instances, Y is greater than or about 40 and less than or about 175, or any subrange as described herein, optionally, about 41 to about 171. In some aspects, AF is about 1 to about 4, or about 1 to about 3 or about 1 to about 2, and HM is about 40 to about 175, or any subrange thereof. Optionally, Y is about 30 to about 185, optionally, about 32 to about 180, HM is about 1 to about 4 and AF is about 30 to about 185. In exemplary aspects, the % ADCC of the antibody composition is within a range defined by Y. Optionally, the % ADCC of the antibody composition is within a range of Y±18. In exemplary aspects, AF is about 1 to about 4. In some aspects, the % high mannose glycans is a value within a range defined by HM, optionally, wherein the range is HM±1.

Optionally, HM is about 1 to about 4. In some instances, the % afucosylated glycans is a value within a range defined by AF optionally, wherein the range is AF±1.

A method of producing an antibody composition within a target range of TAF glycan content is provided wherein said method comprises: (i) measuring the ADCC activity level of a series of samples comprising varying glycoforms of an antibody, (ii) determining the TAF glycan content for each sample of the series, (iii) creating a model which correlates the ADCC activity level to the TAF glycan content, (iv) determining the ADCC activity level for an antibody composition and then calculating a TAF glycan content using the model or determining the TAF glycan content for the antibody composition and calculating the ADCC activity level using the model, and (v) selecting the antibody composition for one or more downstream processing steps when the TAF glycan content calculated in step (iv) is within a target range of TAF glycan content or when the ADCC activity level calculated in step (iv) is within a target range of ADCC activity level. The ADCC activity level in some aspects is measured as essentially described in Example 2. The TAF glycan content in some aspects is measured as essentially described in Example 1. The model may be created by any methods known in the art. In various aspects, the model is a linear regression model and is created as essentially described in Example 3 and/or Example 5.

A method of producing an antibody composition within a target % ADCC range is provided, wherein said method comprises:
i. measuring the % ADCC of a series of samples comprising varying glycoforms of an antibody,
ii. determining the % total afucosylated (TAF) glycans for each sample of the series,
iii. determining a linear equation of a best fit line of a graph which plots for each sample of the series the % ADCC as measured in step (i) as a function of the % TAF glycans as determined in step (ii),
iv. determining the % TAF for an antibody composition and then calculating a % ADCC using the linear equation of step (iii), and
v. selecting the antibody composition for one or more downstream processing steps when the % ADCC calculated in step (iv) is within a target % ADCC range.

Also, a method of producing an antibody composition within a target % TAF range is provided wherein said method comprises:
i. measuring the % ADCC of a series of samples comprising varying glycoforms of an antibody,
ii. determining the % total afucosylated (TAF) glycans for each sample of the series,
iii. determining a linear equation of a best fit line of a graph which plots for each sample of the series the % ADCC as measured in step (i) as a function of the % TAF glycans as determined in step (ii),
iv. determining the % ADCC for an antibody composition and then calculating a % TAF using the linear equation of step (iii), and
v. selecting the antibody composition for one or more downstream processing steps when the % TAF calculated in step (iv) is within a target % TAF range.

Exemplary methods of carrying the first three steps are described in further detail in the Example 3.

The present disclosure further provides a method of producing an antibody composition within a target range for TAF glycan content, comprising determining a target range for TAF glycan content and selecting the antibody composition for one or more downstream processing steps when the TAF glycan content is within the target range for TAF glycan content. In various aspects, the target range for TAF glycan content is m to n, wherein m is [[ADCCₘᵢₙ−y]/x], wherein ADCCₘᵢₙ is the minimum of the target range of ADCC activity level, and n is [[ADCCₘₐₓ−y]/x], wherein ADCCₘₐₓ is the maximum of the target range of ADCC activity level. Optionally, x is about 20.4 to about 27.7 and y is about −11.4 to about 16.7. Alternatively, x is about 9.7 to about 15.2 and y is about −15.6 to about 34.2. In various aspects, the target range for TAF glycan content is m′ to n′, wherein m′ is [ADCCₘᵢₙ/x′], wherein ADCCₘᵢₙ is the minimum of the target range of ADCC activity level, and n′ is [ADCCₘₐₓ]/x′], wherein ADCCₘₐₓ is the maximum of the target range of ADCC activity level. Optionally, x′ is about 24.1 to about 25.4. Alternatively, x′ is about 13.0 to about 13.95.

The present disclosure further provides a method of producing an antibody composition within a target % TAF range said method comprising the following steps: (i) generating a linear equation of a best fit graph by plotting the % ADCC and % TAF glycans of a series of at least 5 reference antibody compositions produced under cell culture conditions, each reference antibody composition having the same amino acid sequence as the antibody composition, (ii) selecting a target % TAF glycan range based on the linear equation generated in step (i) and desired % ADCC activity; (iii) culturing the antibody composition under cell culture conditions; (iv) purifying the antibody composition, (v) sampling the antibody composition to determine the % TAF and (vi) determining whether the % TAF of the antibody composition is within the target % TAF range of step (ii). In exemplary aspects, the method further comprises selecting the antibody composition for one or more downstream processing steps when the % TAF calculated in step (v) is within the target % TAF range.

The present disclosure also provides a method of determining % antibody dependent cellular cytotoxicity (ADCC) of an antibody composition.

In exemplary embodiments, the method comprises:
i. determining the % total afucosylated (TAF) glycans of an antibody composition;
ii. calculating the % ADCC of the antibody composition based on the % TAF using Equation A:

Y=2.6+24.1*X [Equation A],
wherein Y is the % ADCC and X is the % TAF glycans determined in step (i),

A method of determining % antibody dependent cellular cytotoxicity (ADCC) of an antibody composition is furthermore provided. In exemplary embodiments, said method comprises
i. determining the % high mannose glycans and the % afucosylated glycans of an antibody composition,
ii. calculating the % ADCC of the antibody composition based on the % high mannose glycans and the % afucosylated glycans using Equation B:

Y=(0.24+27*HM+22.1*AF) [Equation B],

wherein Y is the % ADCC, HM is the % high mannose glycans determined in step (i), and AF is the % afucosylated glycans determined in step (i), and

In various aspects, the method further comprises selecting the antibody composition for one or more downstream processing steps when Y is within a target % ADCC range.

Processing Steps

The % total afucosylation (TAF) glycans, % high mannose glycans, and/or % afucosylated glycans are determined (e.g., measured) to better inform as to the % antibody-dependent cell-mediated cytotoxicity (ADCC) of the antibody composition. The determining step (e.g., measuring step) may occur at any step during manufacture. In particular, measurements may be taken pre- or post-harvest, at any stage during downstream processing, such as following any chromatography unit operation, including capture chromatography, intermediate chromatography, and/or polish chromatography unit operations; virus inactivation and neutralization, virus filtration; and/or final formulation. The % total afucosylation (TAF) glycans, % high mannose glycans, and/or % afucosylated glycans in various aspects is determined (e.g., measured) in real-time, near real-time, and/or after the fact. Monitoring and measurements can be done using known techniques and commercially available equipment.

In various aspects of the present disclosure, the step of determining (e.g., measuring) the % total afucosylation (TAF) glycans, % high mannose glycans, and/or % afucosylated glycans is carried out after a harvest step. As used herein the term “harvest” refers to the step during which cell culture media containing the recombinant protein of interest is collected and separated at least from the cells of the cell culture. Harvest can be performed continuously. The harvest in some aspects is performed using centrifugation and can further comprise precipitation, filtration, and the like. In various aspects, the determining step is carried out after a chromatography step, optionally, a Protein A chromatography step. In various aspects, the determining step is carried out after harvest and after a chromatography step, e.g., a Protein A chromatography step.

With regard to the presently disclosed methods, the antibody composition in various aspects is selected or chosen for further processing steps, e.g., for one or more downstream processing steps, and the selection is based on a particular parameter, e.g., % ADCC, % total afucosylation (TAF) glycans, % high mannose glycans, and/or % afucosylated glycans. In various instances, the presently disclosed methods comprise using the antibody composition in further processing steps, e.g., in one or more downstream processing steps, based on a particular parameter, e.g., based on the % ADCC, % total afucosylation (TAF) glycans, % high mannose glycans, and/or % afucosylated glycans. In various instances, the presently disclosed methods comprise carrying out further processing steps, e.g., one or more downstream processing steps, with the antibody composition, based on a particular parameter, e.g., based on the % ADCC, % total afucosylation (TAF) glycans, % high mannose glycans, and/or % afucosylated glycans.

In exemplary instances the one or more downstream processing steps is any processing step which occurs after (or downstream of) the processing step at which the % total afucosylation (TAF) glycans, % high mannose glycans, and/or % afucosylated glycans are determined (e.g., measured). For instance, if the % total afucosylation (TAF) glycans, % high mannose glycans, and/or % afucosylated glycans were determined (e.g., measured). For example, if the % total afucosylation (TAF) glycans, % high mannose glycans, and/or % afucosylated glycans were determined (e.g., measured) at harvest, then the one or more downstream processing steps is any processing step which occurs after (or downstream of) the harvest step, which in various aspects comprise(s): a dilution step, a filling step, a filtration step, a formulation step, a chromatography step, a viral filtration step, a viral inactivation step, or a combination thereof. Also, for example, if the % total afucosylation (TAF) glycans, % high mannose glycans, and/or % afucosylated glycans were determined (e.g., measured) after a chromatograph step, e.g., a Protein A chromatography step, then the one or more downstream processing steps is any processing step which occurs after (or downstream of) the chromatography step, which in various aspects comprise(s): a dilution step, a filling step, a filtration step, a formulation step, a further chromatography step, a viral filtration step, a viral inactivation step, or a combination thereof. In exemplary instances the further chromatography step is an ion exchange chromatography step (e.g., a cation exchange chromatography step or an anion exchange chromatography step).

Stages/types of chromatography used during downstream processing include capture or affinity chromatography which is used to separate the recombinant product from other proteins, aggregates, DNA, viruses and other such impurities. In exemplary instances, an initial chromatography step is carried out with Protein A (e.g., Protein A attached to a resin). Intermediate and polish chromatography in various aspects further purify the recombinant protein, removing bulk contaminants, adventitious viruses, trace impurities, aggregates, isoforms, etc. The chromatography can either be performed in bind and elute mode, where the recombinant protein of interest is bound to the chromatography medium and the impurities flow through, or in flow-through mode, where the impurities are bound and the recombinant protein flows through. Examples of such chromatography methods include ion exchange chromatography (IEX), such as anion exchange chromatography (AEX) and cation exchange chromatography (CEX); hydrophobic interaction chromatography (HIC); mixed modal or multimodal chromatography (MM), hydroxyapatite chromatography (HA); reverse phase chromatography and gel filtration.

In various aspects, the downstream step is a viral inactivation step. Enveloped viruses have a capsid enclosed by a lipoprotein membrane or “envelope” and are therefore susceptible to inactivation.

The virus inactivation step in various instances includes heat inactivation/pasteurization, pH inactivation, UV and gamma ray irradiation, use of high intensity broad spectrum white light, addition of chemical inactivating agents, surfactants, and solvent/detergent treatments.

In various aspects, the downstream step is a virus filtration step. In various aspects, the virus filtration step comprises removing non-enveloped viruses. In various aspects, the virus filtration step comprises the use of micro- or nano-filters.

In various aspects, the downstream processing step comprises one or more formulation steps. Following completion of the chromatography steps, the purified recombinant proteins are in various aspects buffer exchanged into a formulation buffer. In exemplary aspects, the buffer exchange is performed using ultrafiltration and diafiltration (UF/DF). In exemplary aspects, the recombinant protein is buffer exchanged into a desired formulation buffer using diafiltration and concentrated to a desired final formulation concentration using ultrafiltration. Additional stability-enhancing excipients in various aspects are added following a UF/DF formulation step.

Recombinant Glycosylated Proteins

The presently disclosed methods relate to composition comprising a recombinant glycosylated protein. In various aspects, the recombinant glycosylated protein comprises an amino acid sequence comprising one or more N-glycosylation consensus sequences of the formula:

Asn-Xaa₁-Xaa₂

wherein Xaa₁ is any amino acid except Pro, and Xaa₂ is Ser or Thr.

In exemplary embodiments, the recombinant glycosylated protein comprises a fragment crystallizable (Fc) polypeptide. The term “Fc polypeptide” as used herein includes native and mutein forms of polypeptides derived from the Fc region of an antibody. Truncated forms of such polypeptides containing the hinge region that promotes dimerization also are included. Fusion proteins comprising Fc moieties (and oligomers formed therefrom) offer the advantage of facile purification by affinity chromatography over Protein A or Protein G columns. In exemplary embodiments, the recombinant glycosylated protein comprises the Fc of an IgG, e.g., a human IgG. In exemplary aspects, the recombinant glycosylated protein comprises the Fc an IgG1 or IgG2. In exemplary aspects, the recombinant glycosylated protein is an antibody, an antibody protein product, a peptibody, or a Fc-fusion protein.

In exemplary aspects, the recombinant glycosylated protein is an antibody. As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. See, e.g., Janeway et al., “Structure of the Antibody Molecule and the Immunoglobulin Genes”, Immunobiology: The Immune System in Health and Disease, 4^{th} ed. Elsevier Science Ltd./Garland Publishing, (1999).

Briefly, in an antibody scaffold, the CDRs are embedded within a framework in the heavy and light chain variable region where they constitute the regions largely responsible for antigen binding and recognition. A variable region comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra).

Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

In various aspects, the antibody can be a monoclonal antibody or a polyclonal antibody. In exemplary instances, the antibody is a mammalian antibody, e.g., a mouse antibody, rat antibody, rabbit antibody, goat antibody, horse antibody, chicken antibody, hamster antibody, pig antibody, human antibody, and the like. In certain aspects, the recombinant glycosylated protein is a monoclonal human antibody.

An antibody, in various aspects, is cleaved into fragments by enzymes, such as, e.g., papain and pepsin. Papain cleaves an antibody to produce two Fab fragments and a single Fc fragment. Pepsin cleaves an antibody to produce a F(ab′)₂ fragment and a pFc′ fragment. In exemplary aspects, the recombinant glycosylated protein is an antibody fragment, e.g., a Fab, Fc, F(ab′)₂, or a pFc′, that retains at least one glycosylation site. With regard to the methods of the disclosure, the antibody may lack certain portions of an antibody, and may be an antibody fragment. In various aspects, the antibody fragment comprises a glycosylation site. In some aspects, the fragment is a “Glycosylated Fc Fragment” which comprises at least a portion of the Fc region of an antibody which is glycosylated post-translationally in eukaryotic cells. In various instances, the recombinant glycosylated protein is glycosylated Fc fragment.

The architecture of antibodies has been exploited to create a growing range of alternative antibody formats that spans a molecular-weight range of at least or about 12-150 kDa and a valency (n) range from monomeric (n=1), dimeric (n=2) and trimeric (n=3) to tetrameric (n=4) and potentially higher; such alternative antibody formats are referred to herein as “antibody protein products” or “antibody binding proteins”.

Antibody protein products can be an antigen binding format based on antibody fragments, e.g., scFvs, Fabs and VHH/VH, which retain full antigen-binding capacity. The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab are widely used fragments that can be easily produced in prokaryotic hosts. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ^{˜}15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4(5): 586-591 (2012).

Other antibody protein products include a single chain antibody (SCA); a diabody; a triabody; a tetrabody; bispecific or trispecific antibodies, and the like. Bispecific antibodies can be divided into five major classes: BslgG, appended IgG, BsAb fragments, bispecific fusion proteins and BsAb conjugates. See, e.g., Spiess et al., Molecular Immunology 67(2) Part A: 97-106 (2015).

In exemplary aspects, the recombinant glycosylated protein comprises any one of these antibody protein products (e.g., scFv, Fab VHH/VH, Fv fragment, ds-scFv, scFab, dimeric antibody, multimeric antibody (e.g., a diabody, triabody, tetrabody), miniAb, peptibody VHH/VH of camelid heavy chain antibody, sdAb, diabody; a triabody; a tetrabody; a bispecific or trispecific antibody, BslgG, appended IgG, BsAb fragment, bispecific fusion protein, and BsAb conjugate) and comprises one or more N-glycosylation consensus sequences, optionally, one or more Fc polypeptides. In various aspects, the antibody protein product comprises a glycosylation site. In exemplary aspects, an antibody protein product can be a Glycosylated Fc Fragment conjugated to an antibody binding fragment (“Glycosylated Fc Fragment antibody product”).

The recombinant glycosylated protein may be an antibody protein product in monomeric form, or polymeric, oligomeric, or multimeric form. In certain embodiments in which the antibody comprises two or more distinct antigen binding regions fragments, the antibody is considered bispecific, trispecific, or multi-specific, or bivalent, trivalent, or multivalent, depending on the number of distinct epitopes that are recognized and bound by the antibody.

In various aspects, the recombinant glycosylated protein is a chimeric antibody or a humanized antibody. The term “chimeric antibody” is used herein to refer to an antibody containing constant domains from one species and the variable domains from a second, or more generally, containing stretches of amino acid sequence from at least two species. The term “humanized” when used in relation to antibodies refers to antibodies having at least CDR regions from a non-human source which are engineered to have a structure and immunological function more similar to true human antibodies than the original source antibodies. For example, humanizing can involve grafting CDR from a non-human antibody, such as a mouse antibody, into a human antibody. Humanizing also can involve select amino acid substitutions to make a non-human sequence look more like a human sequence.

In exemplary aspects, the antibody of the antibody composition binds to an antigen comprising only one antibody binding site, and, optionally, the ADCC activity level of the antibody composition is about 13.5%±0.5% for every 1% TAF present in the antibody composition. In various aspects, the antibody of the antibody composition binds to an antigen comprising only two antibody binding sites, and, optionally, the ADCC activity level of the antibody composition is about 24.74%±0.625% for every 1% TAF present in the antibody composition, In exemplary aspects, the ADCC activity level of the antibody composition is about 12%±1.5%*Q for every 1% TAF present in the antibody composition, Q is the number of antibody binding sites present on the antigen. In exemplary instances, Q is 1 and optionally the antibody is infliximab or a biosimilar thereof. Optionally, Q is 2 and optionally the antibody is rituximab or a biosimilar thereof. In various instances, Q is 3 and thus the ADCC activity level of the antibody composition is about 36% to about 40.5% for every 1% TAF glycan content present in the antibody composition. Also, in some instances, Q is 4 and thus the ADCC activity level of the antibody composition is about 48% to about 54% for every 1% TAF glycan content present in the antibody composition.

Advantageously, the methods are not limited to the antigen-specificity of the antibody, glycosylated Fc fragment, antibody protein product, chimeric antibody, or humanized antibody. Accordingly, the antibody, glycosylated Fc fragment, antibody protein product, chimeric antibody, or humanized antibody has any binding specificity for virtually any antigen. In exemplary aspects, the antibody binds to a hormone, growth factor, cytokine, a cell-surface receptor, or any ligand thereof. In exemplary aspects, the antibody binds to a protein expressed on the cell surface of an immune cell. In exemplary aspects, the antibody binds to a cluster of differentiation molecule selected from the group consisting of: CD1a, CD1b, CD1c, CD1d, CD2, CD3, CD4, CD5, CD6, CD7, CD8, CD9, CD10, CD11A, CD11B, CD11C, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD68, CD69, CD70, CD71, CD72, CD73, CD74, CD75, CD76, CD79a, CD79p, CD80, CD81, CD82, CD83, CDw84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CD109, CD114, CD 115, CD116, CD117, CD118, CD119, CD120a, CD120b, CD121a, CDw121b, CD122, CD123, CD124, CD125, CD126, CD127, CDw128, CD129, CD130, CDw131, CD132, CD134, CD135, CDw136, CDw137, CD138, CD139, CD140a, CD140b, CD141, CD142, CD143, CD144, CD145, CD146, CD147, CD148, CD150, CD151, CD152, CD153, CD154, CD155, CD156, CD157, CD158a, CD158b, CD161, CD162, CD163, CD164, CD165, CD166, and CD182.

In exemplary aspects, the antibody, glycosylated Fc fragment, antibody protein product, chimeric antibody, or humanized antibody is one of those described in U.S. Pat. No. 7,947,809 and U.S. Patent Application Publication No. 20090041784 (glucagon receptor), U.S. Pat. Nos. 7,939,070, 7,833,527, 7,767,206, and 7,786,284 (IL-17 receptor A), U.S. Pat. Nos. 7,872,106 and 7,592,429 (Sclerostin), U.S. Pat. Nos. 7,871,611, 7,815,907, 7,037,498, 7,700,742, and U.S. Patent Application Publication No. 20100255538 (IGF-1 receptor), U.S. Pat. No. 7,868,140 (B7RP1), U.S. Pat. No. 7,807,159 and U.S. Patent Application Publication No. 20110091455 (myostatin), U.S. Pat. Nos. 7,736,644, 7,628,986, 7,524,496, and U.S. Patent Application Publication No. 20100111979 (deletion mutants of epidermal growth factor receptor), U.S. Pat. No. 7,728,110 (SARS coronavirus), U.S. Pat. No. 7,718,776 and U.S. Patent Application Publication No. 20100209435 (OPGL), U.S. Pat. Nos. 7,658,924 and 7,521,053 (Angiopoietin-2), U.S. Pat. Nos. 7,601,818, 7,795,413, U.S. Patent Application Publication No. 20090155274, U.S. Patent Application Publication No. 20110040076 (NGF), U.S. Pat. No. 7,579,186 (TGF-β type II receptor), U.S. Pat. No. 7,541,438 (connective tissue growth factor), U.S. Pat. No. 7,438,910 (IL1-R1), U.S. Pat. No. 7,423,128 (properdin), U.S. Pat. Nos. 7,411,057, 7,824,679, 7,109,003, 6,682,736, 7,132,281, and 7,807,797 (CTLA-4), U.S. Pat. Nos. 7,084,257, 7,790,859, 7,335,743, 7,084,257, and U.S. Patent Application Publication No. 20110045537 (interferon-gamma), U.S. Pat. No. 7,932,372 (MAdCAM), U.S. Pat. No. 7,906,625, U.S. Patent Application Publication No. 20080292639, and U.S. Patent Application Publication No. 20110044986 (amyloid), U.S. Pat. Nos. 7,815,907 and 7,700,742 (insulin-like growth factor I), U.S. Pat. Nos. 7,566,772 and 7,964,193 (interleukin-1p), U.S. Pat. Nos. 7,563,442, 7,288,251, 7,338,660, 7,626,012, 7,618,633, and U.S. Patent Application Publication No. 20100098694 (CD40), U.S. Pat. No. 7,498,420 (c-Met), U.S. Pat. Nos. 7,326,414, 7,592,430, and 7,728,113 (M-CSF), U.S. Pat. Nos. 6,924,360, 7,067,131, and 7,090,844 (MUC18), U.S. Pat. Nos. 6,235,883, 7,807,798, and U.S. Patent Application Publication No. 20100305307 (epidermal growth factor receptor), U.S. Pat. Nos. 6,716,587, 7,872,113, 7,465,450, 7,186,809, 7,317,090, and 7,638,606 (interleukin-4 receptor), U.S. Patent Application Publication No. 20110135657 (BETA-KLOTHO), U.S. Pat. Nos. 7,887,799 and 7,879,323 (fibroblast growth factor-like polypeptides), U.S. Pat. No. 7,867,494 (IgE), U.S. Patent Application Publication No. 20100254975 (ALPHA-4 BETA-7), U.S. Patent Application Publication No. 20100197005 and U.S. Pat. No. 7,537,762 (ACTIVIN RECEPTOR-LIKE KINASE-1), U.S. Pat. No. 7,585,500 and U.S. Patent Application Publication No. 20100047253 (IL-13), U.S. Patent Application Publication No. 20090263383 and U.S. Pat. No. 7,449,555 (CD148), U.S. Patent Application Publication No. 20090234106 (ACTIVIN A), U.S. Patent Application Publication No. 20090226447 (angiopoietin-1 and angiopoietin-2), U.S. Patent Application Publication No. 20090191212 (Angiopoietin-2), U.S. Patent Application Publication No. 20090155164 (C-FMS), U.S. Pat. No. 7,537,762 (activin receptor-like kinase-1), U.S. Pat. No. 7,371,381 (galanin), U.S. Patent Application Publication No. 20070196376 (INSULIN-LIKE GROWTH FACTORS), U.S. Pat. Nos. 7,267,960 and 7,741,115 (LDCAM), U.S. Pat. No. 7,265,212 (CD45RB), U.S. Pat. No. 7,709,611, U.S. Patent Application Publication No. 20060127393 and U.S. Patent Application Publication No. 20100040619 (DKK1), U.S. Pat. No. 7,807,795, U.S. Patent Application Publication No. 20030103978 and U.S. Pat. No. 7,923,008 (osteoprotegerin), U.S. Patent Application Publication No. 20090208489 (OV064), U.S. Patent Application Publication No. 20080286284 (PSMA), U.S. Pat. No. 7,888,482, U.S. Patent Application Publication No. 20110165171, and U.S. Patent Application Publication No. 20110059063 (PAR2), U.S. Patent Application Publication No. 20110150888 (HEPCIDIN), U.S. Pat. No. 7,939,640 (B7L-1), U.S. Pat. No. 7,915,391 (c-Kit), U.S. Pat. Nos. 7,807,796, 7,193,058, and U.S. Pat. No. 7,427,669 (ULBP), U.S. Pat. Nos. 7,786,271, 7,304,144, and U.S. Patent Application Publication No. 20090238823 (TSLP), U.S. Pat. No. 7,767,793 (SIGIRR), U.S. Pat. No. 7,705,130 (HER-3), U.S. Pat. No. 7,704,501 (ataxin-1-like polypeptide), U.S. Pat. Nos. 7,695,948 and 7,199,224 (TNF-α converting enzyme), U.S. Patent Application Publication No. 20090234106 (ACTIVIN A), U.S. Patent Application Publication No. 20090214559 and U.S. Pat. No. 7,438,910 (IL1-R1), U.S. Pat. No. 7,579,186 (TGF-β type II receptor), U.S. Pat. No. 7,569,387 (TNF receptor-like molecules), U.S. Pat. No. 7,541,438, (connective tissue growth factor), U.S. Pat. No. 7,521,048 (TRAIL receptor-2), U.S. Pat. Nos. 6,319,499, 7,081,523, and U.S. Patent Application Publication No. 20080182976 (erythropoietin receptor), U.S. Patent Application Publication No. 20080166352 and U.S. Pat. No. 7,435,796 (B7RP1), U.S. Pat. No. 7,423,128 (properdin), U.S. Pat. Nos. 7,422,742 and 7,141,653 (interleukin-5), U.S. Pat. Nos. 6,740,522 and 7,411,050 (RANKL), U.S. Pat. No. 7,378,091 (carbonic anhydrase IX (CA IX) tumor antigen), U.S. Pat. Nos. 7,318,925 and 7,288,253 (parathyroid hormone), U.S. Pat. No. 7,285,269 (TNF), U.S. Pat. Nos. 6,692,740 and 7,270,817 (ACPL), U.S. Pat. No. 7,202,343 (monocyte chemo-attractant protein-1), U.S. Pat. No. 7,144,731 (SCF), U.S. Pat. Nos. 6,355,779 and 7,138,500 (4-1BB), U.S. Pat. No. 7,135,174 (PDGFD), U.S. Pat. Nos. 6,630,143 and 7,045,128 (Flt-3 ligand), U.S. Pat. No. 6,849,450 (metalloproteinase inhibitor), U.S. Pat. No. 6,596,852 (LERK-5), U.S. Pat. No. 6,232,447 (LERK-6), U.S. Pat. No. 6,500,429 (brain-derived neurotrophic factor), U.S. Pat. No. 6,184,359 (epithelium-derived T-cell factor), U.S. Pat. No. 6,143,874 (neurotrophic factor NNT-1), U.S. Patent Application Publication No. 20110027287 (PROPROTEIN CONVERTASE SUBTILISIN KEXIN TYPE 9 (PCSK9)), U.S. Patent Application Publication No. 20110014201 (IL-18 RECEPTOR), and U.S. Patent Application Publication No. 20090155164 (C-FMS). The above patents and published patent applications are incorporated herein by reference in their entirety for purposes of their disclosure of variable domain polypeptides, variable domain encoding nucleic acids, host cells, vectors, methods of making polypeptides encoding said variable domains, pharmaceutical compositions, and methods of treating diseases associated with the respective target of the variable domain-containing antigen binding protein or antibody.

In exemplary embodiments, the antibody, glycosylated Fc fragment, antibody protein product, chimeric antibody, or humanized antibody is one of Muromonab-CD3 (product marketed with the brand name Orthoclone Okt3®), Abciximab (product marketed with the brand name Reopro®.), Rituximab (product marketed with the brand name MabThera®, Rituxan®), Basiliximab (product marketed with the brand name Simulect®), Daclizumab (product marketed with the brand name Zenapax®), Palivizumab (product marketed with the brand name Synagis®), Infliximab (product marketed with the brand name Remicade®), Trastuzumab (product marketed with the brand name Herceptin®), Alemtuzumab (product marketed with the brand name MabCampath®, Campath-1H©), Adalimumab (product marketed with the brand name Humira®), Tositumomab-I131 (product marketed with the brand name Bexxar®), Efalizumab (product marketed with the brand name Raptiva®), Cetuximab (product marketed with the brand name Erbitux®), l'Ibritumomab tiuxetan (product marketed with the brand name Zevalin®), l'Omalizumab (product marketed with the brand name Xolair®), Bevacizumab (product marketed with the brand name Avastin®), Natalizumab (product marketed with the brand name Tysabri®), Ranibizumab (product marketed with the brand name Lucentis®), Panitumumab (product marketed with the brand name Vectibix®), l'Eculizumab (product marketed with the brand name Soliris®), Certolizumab pegol (product marketed with the brand name Cimzia®), Golimumab (product marketed with the brand name Simponi®), Canakinumab (product marketed with the brand name Ilaris®), Catumaxomab (product marketed with the brand name Removab®), Ustekinumab (product marketed with the brand name Stelara®), Tocilizumab (product marketed with the brand name RoActemra®, Actemra®), Ofatumumab (product marketed with the brand name Arzerra®), Denosumab (product marketed with the brand name Prolia®), Belimumab (product marketed with the brand name Benlysta®), Raxibacumab, Ipilimumab (product marketed with the brand name Yervoy®), and Pertuzumab (product marketed with the brand name Perjeta®). In exemplary embodiments, the antibody is one of anti-TNF alpha antibodies such as adalimumab, infliximab, etanercept, golimumab, and certolizumab pegol; anti-IL1.beta. antibodies such as canakinumab; anti-IL12/23 (p40) antibodies such as ustekinumab and briakinumab; and anti-IL2R antibodies, such as daclizumab.

In exemplary aspects, the antibody binds to a tumor associated antigen and is an anti-cancer antibody. Examples of suitable anti-cancer antibodies include, but are not limited to, anti-BAFF antibodies such as belimumab; anti-CD20 antibodies such as rituximab; anti-CD22 antibodies such as epratuzumab; anti-CD25 antibodies such as daclizumab; anti-CD30 antibodies such as iratumumab, anti-CD33 antibodies such as gemtuzumab, anti-CD52 antibodies such as alemtuzumab; anti-CD152 antibodies such as ipilimumab; anti-EGFR antibodies such as cetuximab; anti-HER2 antibodies such as trastuzumab and pertuzumab; anti-IL6 antibodies, such as siltuximab; and anti-VEGF antibodies such as bevacizumab; anti-IL6 receptor antibodies such as tocilizumab.

In exemplary aspects, the tumor associated antigen is CD20 and the antibody is an anti-CD20 antibody, e.g., an anti-CD20 monoclonal antibody. In exemplary aspects, the tumor associated antigen comprises SEQ ID NO: 3. In exemplary instances, the antibody comprises an amino acid sequence of SEQ ID NO: 1 and an amino acid sequence of SEQ ID NO: 2. In various aspects, the IgG1 antibody is rituximab, or a biosimilar thereof. The term rituximab refers to an IgG1 kappa chimeric murine/human, monoclonal antibody that binds CD20 antigen (see CAS Number: 174722-31-7; DrugBank—DB00073; Kyoto Encyclopedia of Genes and Genomes (KEGG) entry D02994). In exemplary aspects, the antibody comprises a light chain comprising a CDR1, CDR2, and CDR3 as set forth in Table A. In exemplary aspects, the antibody comprises a heavy chain comprising a CDR1, CDR2, and CDR3 as set forth in Table A. In various instances, the antibody comprises the VH and VL or comprising VH-IgG1 and VL-IgG kappa sequences recited in Table A.

TABLE A
Rituximab Amino Acid Sequences

| Description | Sequence | SEQ ID NO: |
|---|---|---|
| LC CDR1 | RASSSVSYIH | 4 |
| LC CDR2 | ATSNLAS | 5 |
| LC CDR3 | QQWTSNPPT | 6 |
| HC CDR1 | SYNMH | 7 |
| HC CDR2 | AIYPGNGDTSYNQKFKG | 8 |
| HC CDR3 | STYYGGDWYFNV | 9 |
| VL | QIVLSQSPAILSASPGEKVTMTCRASSSVSYIHWFQQKPGSSPKPWIYATSNLAS | 10 |
| | GVPVRFSGSGSGTSYSLTISRVEAEDAATYYCQQWTSNPPTFGGGTKLEIK | |
| VH | QVQLQQPGAELVKPGASVKMSCKASGYTFTSYNMHWVKQTPGRGLEWIGAI | 11 |
| | YPGNGDTSYNQKFKGKATLTADKSSSTAYMQLSSLTSEDSAVYYCARSTYYGGD | |
| | WYFNVWGAGTTVTVSA | |
| VL-IgG | QIVLSQSPAILSASPGEKVTMTCRASSSVSYIHWFQQKPGSSPKPWIYATSNLAS | 12 |
| Kappa | GVPVRFSGSGSGTSYSLTISRVEAEDAATYYCQQWTSNPPTFGGGTKLEIKRTV | |
| | AAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFNRGEC | |
| VH-IgG1 | QVQLQQPGAELVKPGASVKMSCKASGYTFTSYNMHWVKQTPGRGLEWIGAI | 13 |
| | YPGNGDTSYNQKFKGKATLTADKSSSTAYMQLSSLTSEDSAVYYCARSTYYGGD | |
| | WYFNVWGAGTTVTVSAASTKGPSVFPLAPSSKSTSGGTAALGCLVKDYFPEPV | |
| | TVSWNSGALTSGVHTFPAVLQSSGLYSLSSVVTVPSSSLGTQTYICNVNHKPSNT | |
| | KVDKKAEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSRDELTKNQVSLTCLVK | |
| | GFYPSDIAVEWESNGQPENNYKTTPPVLDSDGSFFLYSKLTVDKSRWQQGNVF | |
| | SCSVMHEALHNHYTQKSLSLSPGK | |

LC, light chain; HC, heavy chain; VL, variable light chain; VH, variable heavy chain.

In various aspects, the antibody comprises:
i. a light chain (LC) CDR1 comprising an amino acid sequence of SEQ ID NO: 4 or an amino acid sequence which is at least 90% (e.g., at least 95%, at least 96%, at least 97%, at least 98% or at least 99%) identical to SEQ ID NO: 4 or a variant amino acid sequence of SEQ ID NO: 4 with 1 or 2 amino acid substitutions,
ii. a LC CDR2 comprising an amino acid sequence of SEQ ID NO: 5 or an amino acid sequence which is at least 90% (e.g., at least 95%, at least 96%, at least 97%, at least 98% or at least 99%) identical to SEQ ID NO: 5 or a variant amino acid sequence of SEQ ID NO: 5 with 1 or 2 amino acid substitutions,
iii. a LC CDR3 comprising an amino acid sequence of SEQ ID NO: 6 or an amino acid sequence which is at least 90% (e.g., at least 95%, at least 96%, at least 97%, at least 98% or at least 99%) identical to SEQ ID NO: 6 or a variant amino acid sequence of SEQ ID NO: 6 with 1 or 2 amino acid substitutions,
iv. a heavy chain (HC) CDR1 comprising an amino acid sequence of SEQ ID NO: 7 or an amino acid sequence which is at least 90% (e.g., at least 95%, at least 96%, at least 97%, at least 98% or at least 99%) identical to SEQ ID NO: 7 or a variant amino acid sequence of SEQ ID NO: 7 with 1 or 2 amino acid substitutions;
v. a HC CDR2 comprising an amino acid sequence of SEQ ID NO: 8 or an amino acid sequence which is at least 90% (e.g., at least 95%, at least 96%, at least 97%, at least 98% or at least 99%) identical to SEQ ID NO: 8 or a variant amino acid sequence of SEQ ID NO: 8 with 1 or 2 amino acid substitutions;
vi. a HC CDR3 comprising an amino acid sequence of SEQ ID NO: 9 or an amino acid sequence which is at least 90% (e.g., at least 95%, at least 96%, at least 97%, at least 98% or at least 99%) identical to SEQ ID NO: 9 or a variant amino acid sequence of SEQ ID NO: 9 with 1 or 2 amino acid substitutions.

In various instances, the antibody comprises: a LC variable region comprising an amino acid sequence of SEQ ID NO: 10, an amino acid sequence which is at least 90% (e.g., at least 95%, at least 96%, at least 97%, at least 98% or at least 99%) identical to SEQ ID NO: 10, or a variant amino acid sequence of SEQ ID NO: 10 with 1 to 10 (e.g., 1 to 9, 1 to 8, 1 to 7, 1 to 6, 1 to 5, 1 to 4, 1 to 3, 1 or 2) amino acid substitutions.

In exemplary aspects, the antibody comprises: a HC variable region comprising an amino acid sequence of SEQ ID NO: 11, an amino acid sequence which is at least 90% (e.g., at least 95%, at least 96%, at least 97%, at least 98% or at least 99%) identical to SEQ ID NO: 11, or a variant amino acid sequence of SEQ ID NO: 11 with 1 to 10 (e.g., 1 to 9, 1 to 8, 1 to 7, 1 to 6, 1 to 5, 1 to 4, 1 to 3, 1 or 2) amino acid substitutions.

In exemplary instances, the antibody comprises a light chain comprising an amino acid sequence of SEQ ID NO: 12, an amino acid sequence which is at least 90% (e.g., at least 95%, at least 96%, at least 97%, at least 98% or at least 99%) identical to SEQ ID NO: 12, or a variant amino acid sequence of SEQ ID NO: 12 with 1 to 10 (e.g., 1 to 9, 1 to 8, 1 to 7, 1 to 6, 1 to 5, 1 to 4, 1 to 3, 1 or 2) amino acid substitutions.

In various aspects, the antibody comprises a heavy chain comprising an amino acid sequence of SEQ ID NO: 13, an amino acid sequence which is at least 90% (e.g., at least 95%, at least 96%, at least 97%, at least 98% or at least 99%) identical to SEQ ID NO: 13, or a variant amino acid sequence of SEQ ID NO: 13 with 1 to 10 (e.g., I to 9, 1 to 8, 1 to 7, 1 to 6, 1 to 5, 1 to 4, 1 to 3, 1 or 2) amino acid substitutions.

In exemplary aspects, the antigen of the antibody is TNFα and the antibody is an anti-TNFα antibody (which may also be referred to as simply an “anti-TNF” antibody for conciseness), e.g., an anti-TNFα monoclonal antibody. In exemplary aspects, the antigen of the antibody comprises SEQ ID NO: 14. In various aspects, the IgG1 antibody is infliximab or a biosimilar thereof. The term infliximab refers to a chimeric, monoclonal IgG1 kappa antibody composed of human constant and murine variable regions and binds TNFα antigen (See CAS Number: 170277-31-3, DrugBank Accession No. DB00065). Infliximab, also known as chimeric antibody cA2, was derived from a murine monoclonal antibody called A2 (Knight et al., Molec Immunol 30(16): 1443-1453 (1993)). The variable region of the cA2 light chain and of the cA2 light chain are published in International Publication No. WO 2006/065975. In exemplary aspects, the antibody comprises a light chain comprising a CDR1, CDR2, and CDR3 of the variable region of the infliximab light chain as set forth in Table B. In exemplary aspects, the antibody comprises a heavy chain comprising a CDR1, CDR2, and CDR3 of the variable region of the infliximab heavy chain as set forth in Table B. In various instances, the antibody comprises the VH and VL or comprising VH-IgG1 and VL-IgG kappa sequences of infliximab.

TABLE B
Infliximab Amino Acid Sequences

| Description | Sequence | SEQ ID NO: |
|---|---|---|
| VL | DILLTQSPAILSVSPGERVSFSCRASQFVGSSIHWYQQRTNGSPRLLIKYASESMS | 15 |
| | GIPSRFSGSGSGTDFTLSINTVESEDIADYYCQQSHSWPFTFGSGTNLEVK | |
| VH | EVKLEESGGGLVQPGGSMKLSCVASGFIFSNHWMNWVRQSPEKGLEWVAEIR | 16 |
| | SKSINSATHYAESVKGRFTISRDDSKSAVYLQMTDLRTEDTGVYYCSRNYYGSTY | |
| | DYWGQGTTLTVS | |

LC, light chain; HC, heavy chain; VL, variable light chain; VH, variable heavy chain.

In various instances, the antibody comprises: a LC variable region comprising an amino acid sequence of SEQ ID NO: 15, an amino acid sequence which is at least 90% (e.g., at least 95%, at least 96%, at least 97%, at least 98% or at least 99%) identical to SEQ ID NO: 15, or a variant amino acid sequence of SEQ ID NO: 15 with 1 to 10 (e.g., 1 to 9, 1 to 8, 1 to 7, 1 to 6, 1 to 5, 1 to 4, 1 to 3, 1 or 2) amino acid substitutions. In exemplary aspects, the antibody comprises: a HC variable region comprising an amino acid sequence of SEQ ID NO: 16, an amino acid sequence which is at least 90% (e.g., at least 95%, at least 96%, at least 97%, at least 98% or at least 99%) identical to SEQ ID NO: 16, or a variant amino acid sequence of SEQ ID NO: 16 with 1 to 10 (e.g., 1 to 9, 1 to 8, 1 to 7, 1 to 6, 1 to 5, 1 to 4, 1 to 3, 1 or 2) amino acid substitutions.

Compositions

The presently disclosed methods relate to compositions comprising recombinant glycosylated proteins. In various aspects, the composition comprises only one type of recombinant glycosylated protein. In various instances, the composition comprises recombinant glycosylated proteins wherein each recombinant glycosylated protein of the composition comprises the same or essentially the amino acid sequence. In various aspects, the composition comprises recombinant glycosylated proteins wherein each recombinant glycosylated protein of the composition comprises an amino acid sequence which is at least 90% identical to the amino acid sequences of all other recombinant glycosylated proteins of the composition. In various aspects, the composition comprises recombinant glycosylated proteins wherein each recombinant glycosylated protein of the composition comprises an amino acid sequence which is at least 95%, at least 96%, at least 97%, at least 98%, or at least 99% identical to the amino acid sequences of all other recombinant glycosylated proteins of the composition. In various aspects, the composition comprises recombinant glycosylated proteins wherein each recombinant glycosylated protein of the composition comprises an amino acid sequence which is the same or essentially the same (e.g., at least 90% or at least 95%, at least 96%, at least 97%, at least 98%, or at least 99% identical to the amino acid sequences of all other recombinant glycosylated proteins of the composition) but the glycoprofiles of the recombinant glycosylated proteins of the composition may differ from each other.

In exemplary aspects, the recombinant glycosylated protein is an antibody fragment and accordingly, the composition may be an antibody fragment composition.

In exemplary aspects, the recombinant glycosylated protein is an antibody protein product and accordingly, the composition may be an antibody protein product composition.

In exemplary aspects, the recombinant glycosylated protein is a Glycosylated Fc Fragment and accordingly, the composition may be a Glycosylated Fc Fragment composition.

In exemplary aspects, the recombinant glycosylated protein is a Glycosylated Fc Fragment antibody product and accordingly, the composition may be a Glycosylated Fc Fragment antibody product composition.

In exemplary aspects, the recombinant glycosylated protein is a chimeric antibody and accordingly, the composition may be a chimeric antibody composition.

In exemplary aspects, the recombinant glycosylated protein is a humanized antibody and accordingly, the composition may be a humanized antibody composition.

In exemplary aspects, the recombinant glycosylated protein is an antibody and the composition is an antibody composition. In various aspects, the composition comprises only one type of antibody. In various instances, the composition comprises antibodies wherein each antibody of the antibody composition comprises the same or essentially the amino acid sequence. In various aspects, the antibody composition comprises antibodies wherein each antibody of the antibody composition comprises an amino acid sequence which is at least 90% identical to the amino acid sequences of all other antibodies of the antibody composition. In various aspects, the antibody composition comprises antibodies wherein each antibody of the antibody composition comprises an amino acid sequence which is at least 95%, at least 96%, at least 97%, at least 98%, or at least 99% identical to the amino acid sequences of all other antibodies of the antibody composition. In various aspects, the antibody composition comprises antibodies wherein each antibody of the antibody composition comprises an amino acid sequence which is the same or essentially the same (e.g., at least 90% or at least 95%, at least 96%, at least 97%, at least 98%, or at least 99% identical to the amino acid sequences of all other antibodies of the antibody composition) but the glycoprofiles of the antibodies of the antibody composition may differ from each other. In exemplary aspects, the antibody composition comprises a heterogeneous mixture of different glycoforms of the antibody. In various instances, the antibody composition may be characterized in terms of its TAF glycans content, HM glycans content and/or its AF glycans content. In various aspects, the antibody composition is described in terms of a % TAF glycans, % HM glycans, and/or % afucosylated glycans. Optionally, the antibody composition may be characterized in terms its content of other types of glycans, e.g., galactosylated glycoforms, fucosylated glycoforms, and the like.

In various aspects, each antibody of the antibody composition in an IgG, optionally, an IgG1. In various instances, each antibody of the antibody composition binds to a tumor-associated antigen, e.g., CD20. In various aspects, the CD20 comprises the amino acid sequence of SEQ ID NO: 3. In exemplary aspects, each antibody of the antibody composition is an anti-CD20 antibody. In various aspects, each antibody of the antibody composition comprises:
i. a light chain (LC) CDR1 comprising an amino acid sequence of SEQ ID NO: 4 or an amino acid sequence which is at least 90% (e.g., at least 95%, at least 96%, at least 97%, at least 98% or at least 99%) identical to SEQ ID NO: 4 or a variant amino acid sequence of SEQ ID NO: 4 with 1 or 2 amino acid substitutions,
ii. a LC CDR2 comprising an amino acid sequence of SEQ ID NO: 5 or an amino acid sequence which is at least 90% (e.g., at least 95%, at least 96%, at least 97%, at least 98% or at least 99%) identical to SEQ ID NO: 5 or a variant amino acid sequence of SEQ ID NO: 5 with 1 or 2 amino acid substitutions,
iii. a LC CDR3 comprising an amino acid sequence of SEQ ID NO: 6 or an amino acid sequence which is at least 90% (e.g., at least 95%, at least 96%, at least 97%, at least 98% or at least 99%) identical to SEQ ID NO: 6 or a variant amino acid sequence of SEQ ID NO: 6 with 1 or 2 amino acid substitutions,
iv. a heavy chain (HC) CDR1 comprising an amino acid sequence of SEQ ID NO: 7 or an amino acid sequence which is at least 90% (e.g., at least 95%, at least 96%, at least 97%, at least 98% or at least 99%) identical to SEQ ID NO: 7 or a variant amino acid sequence of SEQ ID NO: 7 with 1 or 2 amino acid substitutions;
v. a HC CDR2 comprising an amino acid sequence of SEQ ID NO: 8 or an amino acid sequence which is at least 90% (e.g., at least 95%, at least 96%, at least 97%, at least 98% or at least 99%) identical to SEQ ID NO: 8 or a variant amino acid sequence of SEQ ID NO: 8 with 1 or 2 amino acid substitutions; and/or
vi. a HC CDR3 comprising an amino acid sequence of SEQ ID NO: 9 or an amino acid sequence which is at least 90% (e.g., at least 95%, at least 96%, at least 97%, at least 98% or at least 99%) identical to SEQ ID NO: 9 or a variant amino acid sequence of SEQ ID NO: 9 with 1 or 2 amino acid substitutions.

In various instances, each antibody of the antibody composition comprises: a LC variable region comprising an amino acid sequence of SEQ ID NO: 10, an amino acid sequence which is at least 90% (e.g., at least 95%, at least 96%, at least 97%, at least 98% or at least 99%) identical to SEQ ID NO: 10, or a variant amino acid sequence of SEQ ID NO: 10 with 1 to 10 (e.g., 1 to 9, 1 to 8, 1 to 7, 1 to 6, 1 to 5, 1 to 4, 1 to 3, 1 or 2) amino acid substitutions.

In exemplary aspects, each antibody of the antibody composition comprises: a HC variable region comprising an amino acid sequence of SEQ ID NO: 11, an amino acid sequence which is at least 90% (e.g., at least 95%, at least 96%, at least 97%, at least 98% or at least 99%) identical to SEQ ID NO: 11, or a variant amino acid sequence of SEQ ID NO: 11 with 1 to 10 (e.g., 1 to 9, 1 to 8, 1 to 7, 1 to 6, 1 to 5, 1 to 4, 1 to 3, 1 or 2) amino acid substitutions.

In exemplary instances, each antibody of the antibody composition comprises a light chain comprising an amino acid sequence of SEQ ID NO: 12, an amino acid sequence which is at least 90% (e.g., at least 95%, at least 96%, at least 97%, at least 98% or at least 99%) identical to SEQ ID NO: 12, or a variant amino acid sequence of SEQ ID NO: 12 with 1 to 10 (e.g., 1 to 9, 1 to 8, 1 to 7, 1 to 6, 1 to 5, 1 to 4, 1 to 3, 1 or 2) amino acid substitutions.

In various aspects, each antibody of the antibody composition comprises a heavy chain comprising an amino acid sequence of SEQ ID NO: 13, an amino acid sequence which is at least 90% (e.g., at least 95%, at least 96%, at least 97%, at least 98% or at least 99%) identical to SEQ ID NO: 13, or a variant amino acid sequence of SEQ ID NO: 13 with 1 to 10 (e.g., 1 to 9, 1 to 8, 1 to 7, 1 to 6, 1 to 5, 1 to 4, 1 to 3, 1 or 2) amino acid substitutions.

In various aspects, each antibody of the antibody composition in an IgG, optionally, an IgG1. In various instances, each antibody of the antibody composition binds to a tumor-associated antigen, e.g., TNFalpha. In various aspects, the TNFalpha comprises the amino acid sequence of SEQ ID NO: 14. In exemplary aspects, each antibody of the antibody composition is an anti-TNFalpha antibody.

In various instances, each antibody of the antibody composition comprises: a LC variable region comprising an amino acid sequence of SEQ ID NO: 15, an amino acid sequence which is at least 90% (e.g., at least 95%, at least 96%, at least 97%, at least 98% or at least 99%) identical to SEQ ID NO: 15, or a variant amino acid sequence of SEQ ID NO: 15 with 1 to 10 (e.g., 1 to 9, 1 to 8, 1 to 7, 1 to 6, 1 to 5, 1 to 4, 1 to 3, 1 or 2) amino acid substitutions.

In exemplary aspects, each antibody of the antibody composition comprises: a HC variable region comprising an amino acid sequence of SEQ ID NO: 16, an amino acid sequence which is at least 90% (e.g., at least 95%, at least 96%, at least 97%, at least 98% or at least 99%) identical to SEQ ID NO: 16, or a variant amino acid sequence of SEQ ID NO: 16 with 1 to 10 (e.g., 1 to 9, 1 to 8, 1 to 7, 1 to 6, 1 to 5, 1 to 4, 1 to 3, 1 or 2) amino acid substitutions.

In exemplary aspects, the antibody composition comprises a heterogeneous mixture of different glycoforms of the antibody. In various instances, the antibody composition may be characterized in terms of its TAF glycans content, HM glycans content and/or its AF glycans content. In various aspects, the antibody composition is described in terms of a % TAF glycans, % HM glycans, and/or % afucosylated glycans. Optionally, the antibody composition may be characterized in terms its content of other types of glycans, e.g., galactosylated glycoforms, fucosylated glycoforms, and the like.

In exemplary aspect, the antibody composition has a % TAF glycans as calculated using Equation A. In exemplary aspects, the antibody composition has a % TAF glycans within a range defined by X of Equation A. In exemplary instances, the % TAF glycans is within X±0.4. In exemplary aspect, the antibody composition has a % TAF glycans as determined (e.g., measured) in the determining step of the presently disclosed methods. In exemplary aspects, the % TAF glycans is determined by hydrophilic interaction chromatography, optionally, by the method described in Example 1. By way of example, the antibody composition in various instances is less than or about 50% (e.g., less than or about 40%, less than or about 30%, less than or about 25%, less than or about 20%, less than or about 15%) TAF glycans. In exemplary aspects, the antibody composition is less than about 10% (e.g., less than or about 9%, less than or about 8%, less than or about 7%, less than or about 6%, less than or about 5%, less than or about 4%, less than or about 3%, less than or about 2%) TAF glycans. In exemplary aspects, the antibody composition is about 4% to about 10% TAF glycans. In exemplary aspects, the antibody composition is about 2% to about 6% TAF glycans. In exemplary aspects, the antibody composition is about 2.5% to about 5% of TAF glycans. In exemplary aspects, the antibody composition is less than or about 4% TAF glycans. In further exemplary aspects, the antibody composition is less than or about 4% and greater than or about 2% TAF glycans. In various aspects, the % TAF glycans is greater than or about 1.55% and less than or about 6.95% or about 1.72% to about 6.74%.

In exemplary aspect, the antibody composition has a % afucosylated glycans as calculated using to Equation B. In exemplary aspects, the antibody composition has a % afucosylated glycans within a range defined by AF of Equation B. In exemplary instances, the % afucosylated glycans is within AF±1. In exemplary aspect, the antibody composition has a % afucosylated glycans as determined (e.g., measured) in the determining step of the presently disclosed methods. In exemplary aspects, the % afucosylated glycans is determined by hydrophilic interaction chromatography, optionally, by the method described in Example 1. By way of example, the antibody composition in various instances is less than or about 5% afucosylated glycans. In exemplary aspects, the % afucosylated glycans is about 1 to about 4. In exemplary aspects, the antibody composition is less than or about 4% afucosylated glycans. In exemplary aspects, the antibody composition is less than or about 3.5% afucosylated glycans.

In exemplary aspect, the antibody composition has a % high mannose glycans as calculated using Equation B. In exemplary aspects, the antibody composition has a % high mannose glycans within a range defined by HM of Equation B. In exemplary instances, the % high mannose glycans is within HM±1. In exemplary aspect, the antibody composition has a % high mannose glycans as determined (e.g., measured) in the determining step of the presently disclosed methods. In exemplary aspects, the % high mannose glycans is determined by hydrophilic interaction chromatography, optionally, by the method described in Example 1. By way of example, the antibody composition, in exemplary aspects, is less than or about 5% high mannose glycans. In exemplary aspects, the % high mannose glycans is about 1 to about 4. In exemplary aspects, the antibody composition is less than or about 4 high mannose glycans. In exemplary aspects, the antibody composition is less than or about 3.5% high mannose glycans.

In exemplary aspect, the antibody composition has a % ADCC as calculated using Equation A or Equation B. In exemplary aspect, the antibody composition has a % ADCC as determined (e.g., measured) in a determining step. In exemplary aspects, the % ADCC is determined by a quantitative cell-based assay which measures the ability of the antibodies of the antibody composition to mediate cell cytotoxicity in a dose-dependent manner in cells expressing the antigen of the antibodies and engaging Fc-gammaRIIIA receptors on effector cells through the Fc domain of the antibodies, e.g., a method as described in Example 2. By way of example, the antibody composition in various instances is about 40% to about 175% ADCC or about 40% to about 170% ADCC or about 44% to about 165% ADCC. In exemplary aspect, the antibody composition has a % ADCC greater than or about 40 and less than or about 175 or less than or about 170, optionally, about 41 to about 171. In exemplary aspect, the antibody composition has a % ADCC which is about 30 to about 185, optionally, about 32 to about 180. In various aspects, the % ADCC is greater than or about 60 and less than or about 130. In exemplary aspects, the antibody composition has a % ADCC within a range defined by Y of Equation A or Equation B. In various aspects, the % ADCC is within Y±20, e.g., within Y±19, Y±18, or Y±17.

With regard to % TAF glycans, X, and % ADCC, Y, of Equation A, in some aspects, Y is greater than or about 40 and less than or about 170 and X is greater than or about 1.55% and less than or about 6.95%. In various instances, Y is greater than or about 44% and less than or about 165%, and optionally, wherein X is about 1.72% to about 6.74%.

With regard to % afucosylated glycans, AF, and % high mannose glycans, HM, and % ADCC, Y, of Equation B, in some aspects, Y is greater than or about 40 and less than or about 175, optionally, about 41 to about 171, wherein AF is about 1 to about 4 and wherein HM is about 40 to about 175. In various instances, Y is about 30 to about 185, optionally, about 32 to about 180, wherein HM is about 1 to about 4 and wherein AF is about 30 to about 185.

In exemplary embodiments, the composition is combined with a pharmaceutically acceptable carrier, diluent or excipient. Accordingly, provided herein are pharmaceutical compositions comprising the recombinant glycosylated protein composition (e.g., the antibody composition or antibody binding protein composition) described herein and a pharmaceutically acceptable carrier, diluent or excipient. As used herein, the term “pharmaceutically acceptable carrier” includes any of the standard pharmaceutical carriers, such as a phosphate buffered saline solution, water, emulsions such as an oil/water or water/oil emulsion, and various types of wetting agents.

In exemplary embodiments, the antibody composition is produced by glycosylation competent cells in cell culture as described herein.

Additional Steps

The methods disclosed herein, in various aspects, comprise additional steps. For example, in some aspects, the methods comprise one or more upstream steps or downstream steps involved in producing, purifying, and formulating a recombinant glycosylated protein, e.g., an antibody. Optionally, the downstream steps are any one of those downstream processing steps described herein or known in the art. See, e.g., Processing Steps. In exemplary embodiments, the method comprises steps for generating host cells that express a recombinant glycosylated protein (e.g., antibody). The host cells, in some aspects, are prokaryotic host cells, e.g., E. coli or Bacillus subtilis, or the host cells, in some aspects, are eukaryotic host cells, e.g., yeast cells, filamentous fungi cells, protozoa cells, insect cells, or mammalian cells (e.g., CHO cells). Such host cells are described in the art. See, e.g., Frenzel, et al., Front Immunol 4: 217 (2013) and herein under “Cells.” For example, the methods comprise, in some instances, introducing into host cells a vector comprising a nucleic acid comprising a nucleotide sequence encoding the recombinant glycosylated protein, or a polypeptide chain thereof.

In exemplary aspects, the methods comprise maintaining cells, e.g., glycosylation-competent cells in a cell culture. Accordingly, the methods may comprise carrying out any one or more steps described herein in Maintaining Cells In A Cell Culture.

In exemplary embodiments, the methods disclosed herein comprise steps for isolating and/or purifying the recombinant glycosylated protein (e.g., recombinant antibody) from the culture. In exemplary aspects, the method comprises one or more chromatography steps including, but not limited to, e.g., affinity chromatography (e.g., protein A affinity chromatography), ion exchange chromatography, and/or hydrophobic interaction chromatography. In exemplary aspects, the method comprises steps for producing crystalline biomolecules from a solution comprising the recombinant glycosylated proteins.

The methods of the disclosure, in various aspects, comprise one or more steps for preparing a composition, including, in some aspects, a pharmaceutical composition, comprising the purified recombinant glycosylated protein. Such compositions are discussed herein.

Maintaining Cells in a Cell Culture

With regard to the methods of producing an antibody composition of the present disclosure, the antibody composition may be produced by maintaining cells in a cell culture. The cell culture may be maintained according to any set of conditions suitable for production of a recombinant glycosylated protein. For example, in some aspects, the cell culture is maintained at a particular pH, temperature, cell density, culture volume, dissolved oxygen level, pressure, osmolality, and the like. In exemplary aspects, the cell culture prior to inoculation is shaken (e.g., at 70 rpm) at 5% CO₂ under standard humidified conditions in a CO₂ incubator. In exemplary aspects, the cell culture is inoculated with a seeding density of about 10⁶ cells/mL in 1.5 L medium.

In exemplary aspects, the methods of the disclosure comprise maintaining the glycosylation-competent cells in a cell culture medium at a pH of about 6.85 to about 7.05, e.g., in various aspects, about 6.85, about 6.86, about 6.87, about 6.88, about 6.89, about 6.90, about 6.91, about 6.92, about 6.93, about 6.94, about 6.95, about 6.96, about 6.97, about 6.98, about 6.99, about 7.00, about 7.01, about 7.02, about 7.03, about 7.04, or about 7.05.

In exemplary aspects, the methods comprise maintaining the cell culture at a temperature between 302C and 402C. In exemplary embodiments, the temperature is between about 322C to about 382C or between about 352C to about 382C.

In exemplary aspects, the methods comprise maintaining the osmolality between about 200 mOsm/kg to about 500 mOsm/kg. In exemplary aspects, the method comprises maintaining the osmolality between about 225 mOsm/kg to about 400 mOsm/kg or about 225 mOsm/kg to about 375 mOsm/kg. In exemplary aspects, the method comprises maintaining the osmolality between about 225 mOsm/kg to about 350 mOsm/kg. In various aspects, osmolality (mOsm/kg) is maintained at about 200, 225, about 250, about 275, about 300, about 325, about 350, about 375, about 400, about 425, about 450, about 475, or about 500.

In exemplary aspects, the methods comprise maintaining dissolved the oxygen (DO) level of the cell culture at about 20% to about 60% oxygen saturation during the initial cell culture period. In exemplary instances, the method comprises maintaining DO level of the cell culture at about 30% to about 50% (e.g., about 35% to about 45%) oxygen saturation during the initial cell culture period. In exemplary instances, the method comprises maintaining DO level of the cell culture at about 20%, about 25%, about 30%, about 35%, about 40%, about 45%, about 50%, about 55%, or about 60% oxygen saturation during the initial cell culture period. In exemplary aspects, the DO level is about 35 mm Hg to about 85 mmHg or about 40 mm Hg to about 80 mmHg or about 45 mm Hg to about 75 mm Hg.

The cell culture is maintained in any one or more culture medium. In exemplary aspects, the cell culture is maintained in a medium suitable for cell growth and/or is provided with one or more feeding media according to any suitable feeding schedule. In exemplary aspects, the method comprises maintaining the cell culture in a medium comprising glucose, fucose, lactate, ammonia, glutamine, and/or glutamate. In exemplary aspects, the method comprises maintaining the cell culture in a medium comprising manganese at a concentration less than or about 1 μM during the initial cell culture period. In exemplary aspects, the method comprises maintaining the cell culture in a medium comprising about 0.25 μM to about 1 μM manganese. In exemplary aspects, the method comprises maintaining the cell culture in a medium comprising negligible amounts of manganese. In exemplary aspects, the method comprises maintaining the cell culture in a medium comprising copper at a concentration less than or about 50 ppb during the initial cell culture period. In exemplary aspects, the method comprises maintaining the cell culture in a medium comprising copper at a concentration less than or about 40 ppb during the initial cell culture period. In exemplary aspects, the method comprises maintaining the cell culture in a medium comprising copper at a concentration less than or about 30 ppb during the initial cell culture period. In exemplary aspects, the method comprises maintaining the cell culture in a medium comprising copper at a concentration less than or about 20 ppb during the initial cell culture period. In exemplary aspects, the medium comprises copper at a concentration greater than or about 5 ppb or greater than or about 10 ppb. In exemplary aspects, the cell culture medium comprises mannose. In exemplary aspects, the cell culture medium does not comprise mannose.

In exemplary embodiments, the type of cell culture is a fed-batch culture or a continuous perfusion culture. However, the methods of the disclosure are advantageously not limited to any particular type of cell culture.

The cells maintained in cell culture may be glycosylation-competent cells. In exemplary aspects, the glycosylation-competent cells are eukaryotic cells, including, but not limited to, yeast cells, filamentous fungi cells, protozoa cells, algae cells, insect cells, or mammalian cells. Such host cells are described in the art. See, e.g., Frenzel, et al., Front Immunol 4: 217 (2013). In exemplary aspects, the eukaryotic cells are mammalian cells. In exemplary aspects, the mammalian cells are non-human mammalian cells. In some aspects, the cells are Chinese Hamster Ovary (CHO) cells and derivatives thereof (e.g., CHO-K1, CHO pro-3), mouse myeloma cells (e.g., NS0, GS-NS0, Sp2/0), cells engineered to be deficient in dihydrofolatereductase (DHFR) activity (e.g., DUKX-X11, DG44), human embryonic kidney 293 (HEK293) cells or derivatives thereof (e.g., HEK293T, HEK293-EBNA), green African monkey kidney cells (e.g., COS cells, VERO cells), human cervical cancer cells (e.g., HeLa), human bone osteosarcoma epithelial cells U2-OS, adenocarcinomic human alveolar basal epithelial cells A549, human fibrosarcoma cells HT1080, mouse brain tumor cells CAD, embryonic carcinoma cells P19, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human liver cancer cells Hep G2, mouse B myeloma cells J558L, or baby hamster kidney (BHK) cells (Gaillet et al. 2007; Khan, Adv Pharm Bull 3(2): 257-263 (2013)).

Cells that are not glycosylation-competent can also be transformed into glycosylation-competent cells, e.g. by transfecting them with genes encoding relevant enzymes necessary for glycosylation. Exemplary enzymes include but are not limited to oligosaccharyltransferases, glycosidases, glucosidase I, glucosidease II, calnexin/calreticulin, glycosyltransferases, mannosidases, GlcNAc transferases, galactosyltransferases, and sialyltransferases.

In exemplary embodiments, the glycosylation-competent cells are not genetically modified to alter the activity of an enzyme of the de novo pathway or the salvage pathway. These two pathways of fucose metabolism are shown in FIG. 2. In exemplary embodiments, the glycosylation-competent cells are not genetically modified to alter the activity of any one or more of: a fucosyl-transferase (FUT, e.g., FUT1, FUT2, FUT3, FUT4, FUT5, FUT6, FUT7, FUT8, FUT9), a fucose kinase, a GDP-fucose pyrophosphorylase, GDP-D-mannose-4,6-dehydratase (GMD), and GDP-keto-6-deoxymannose-3,5-epimerase, 4-reductase (FX). In exemplary embodiments, the glycosylation-competent cells are not genetically modified to knock-out a gene encoding FX.

In exemplary embodiments, the glycosylation-competent cells are not genetically modified to alter the activity β(1,4)-N-acetylglucosaminyltransferase III (GNTIII) or GDP-6-deoxy-D-lyxo-4-hexulose reductase (RMD). In exemplary aspects, the glycosylation-competent cells are not genetically modified to overexpress GNTIII or RMD.

### EXEMPLARY EMBODIMENTS

Exemplary embodiments of the present disclosure are provided below.
E1 A method of producing an antibody composition, said method comprising:
   i. determining the % total afucosylated (TAF) glycans of an antibody composition;
   ii. calculating a % antibody dependent cellular cytotoxicity (ADCC) of the antibody composition based on the % TAF using Equation A:

Y=2.6+24.1*X [Equation A],
wherein Y is the % ADCC and X is the % TAF glycans determined in step (i), and
iii. selecting the antibody composition for one or more downstream processing steps when Y is within a target % ADCC range.
E2. A method of producing an antibody composition, said method comprising
   i. determining the % high mannose glycans and the % afucosylated glycans of an antibody composition,
   ii. calculating a % antibody dependent cellular cytotoxicity (ADCC) of the antibody composition based on the % high mannose glycans and the % afucosylated glycans using Equation B:

Y=(0.24+27*HM+22.1*AF) [Equation B],
wherein Y is the % ADCC, HM is the % high mannose glycans determined in step (i), and AF is the % afucosylated glycans determined in step (i), and
iii. selecting the antibody composition for one or more downstream processing steps when Y is within a target % ADCC range.
E3. A method of producing an antibody composition with a target % ADCC, said method comprising
   i. calculating a target % total afucosylated (TAF) glycans for the target % ADCC using Equation A:

Y=2.6+24.1*X [Equation A],
wherein Y is the target % ADCC and X is the target % TAF glycans, and
ii. maintaining glycosylation-competent cells in a cell culture to produce an antibody composition with the target % TAF glycans, X.
E4. A method of producing an antibody composition with a target % ADCC, said method comprising
   i. calculating a target % afucosylated glycans and a target % high mannose glycans for the target % ADCC using Equation B:

Y=(0.24+27*HM+22.1*AF) [Equation B],
wherein Y is the target % ADCC, HM is the target % high mannose glycans and AF is the target % afucosylated glycans, and
ii. maintaining glycosylation-competent cells in a cell culture to produce an antibody composition with the target % high mannose glycans and the target % afucosylated glycans.
E5. The method of embodiment 3 or 4, wherein the target % ADCC is within a target % ADCC range.
E6. The method of any one of embodiments 1, 2, and 5, wherein the target % ADCC range is greater than or about 40 and less than or about 170.
E7. The method of embodiment 6, wherein the target % ADCC range is greater than or about 44 and less than or about 165.
E8. The method of embodiment 7, wherein the target % ADCC range ±is greater than or about 60 and less than or about 130.
E9. The method of an± one of embodiments 1-4, wherein the target % ADCC range is Y±20.
E10. The method of embodiment 1 or embodiment 3, wherein the target % ADCC range is Y±17.
E11. The method of embodiment 2 or embodiment 4, wherein the target % ADCC range is Y±18.
E12. A method of producing an antibody composition with a % ADCC, Y, which is optionally greater than or about 40 and less than or about 170, said method comprising
   i. determining the % total afucosylated (TAF) glycans, X, of the antibody composition, and
   ii. selecting the antibody composition for one or more downstream processing steps, when X is equivalent to (Y−2.6)/24.1.
E13. The method of embodiment 13, wherein X is greater than or about 1.55% and less than or about 6.95%.
E14. The method of embodiment 13 or 14, wherein Y is greater than or about 44% and less than or about 165%, and optionally, wherein X is about 1.72% to about 6.74%.
E15. A method of producing an antibody composition with a % ADCC, Y, said method comprising
   i. determining the % total afucosylated (TAF) glycans, X, of the antibody composition, and
   ii. selecting the antibody composition for one or more downstream processing steps, when the X is equivalent to (Y−2.6)/24.1, optionally, wherein X is greater than or about X−0.4 and less than or about X+0.4, and wherein the % ADCC is greater than about Y−17 and less than or about Y+17.
E16. A method of producing an antibody composition with a % ADCC, said method comprising
   i. determining the % afucosylated glycans and the % high mannose glycans of the antibody composition, and
   ii. selecting the antibody composition for one or more downstream processing steps, when AF and HM are related to Y according to Equation B

Y=(0.24+27*HM+22.1*AF) [Equation B],
wherein Y is the % ADCC, HM is the % high mannose glycans determined in step (i), and AF is the % afucosylated glycans determined in step (i).
E17. The method of embodiment 16, wherein Y is greater than or about 40 and less than or about 175, optionally, about 41 to about 171, wherein AF is about 1 to about 4 and wherein HM is about 40 to about 175.
E18. The method of embodiment 16, wherein Y is about 30 to about 185, optionally, about 32 to about 180, wherein HM is about 1 to about 4 and wherein AF is about 30 to about 185.
E19. The method of embodiment 16, wherein the % ADCC of the antibody composition is within a range defined by Y.
E20. The method of embodiment 19, wherein the % ADCC of the antibody composition is within a range of Y±18.
E21. The method of any one of embodiments 16, 19 and 20, wherein AF is about 1 to about 4.
E22. The method of embodiment 21, wherein the % high mannose glycans is a value within a range defined by HM, optionally, wherein the range is HM±1.
E23. The method of any one of embodiments 16, 19 and 20, wherein HM is about 1 to about 4.
E24. The method of embodiment 24, wherein the % afucosylated glycans is a value within a range defined by AF optionally, wherein the range is AF±1.
E25. The method of any one of the preceding embodiments, wherein the % TAF glycans is determined by calculating the sum of the % high mannose glycans and the % afucosylated glycans.
E26. The method of any one of the preceding embodiments, wherein the % high mannose glycans and the % afucosylated glycans are determined by hydrophilic interaction chromatography.
E27. The method of embodiment 26, wherein the % high mannose glycans and the % afucosylated glycans are determined by the method described in Example 1.
E28. The method of any one of the preceding embodiments, wherein the % ADCC is determined by a quantitative cell-based assay which measures the ability of the antibodies of the antibody composition to mediate cell cytotoxicity in a dose-dependent manner in cells expressing the antigen of the antibodies and engaging Fc-gammaRIIIA receptors on effector cells through the Fc domain of the antibodies.
E29. The method of embodiment 28, wherein the % ADCC is determined by the assay described in Example 2.
E30. The method of any one of embodiments 1, 2, and 5-19, wherein the determining step is carried out after a harvest step.
E31. The method of embodiment 30, wherein the determining step is carried out after chromatography step.
E32. The method of embodiment 31, wherein the chromatography step is a Protein A chromatography step.
E33. The method of any one of the preceding embodiments, wherein the one or more downstream processing steps comprise(s): a dilution step, a filling step, a filtration step, a formulation step, a chromatography step, a viral filtration step, a viral inactivation step, or a combination thereof.
E34. The method of embodiment 33, wherein the chromatography step is an ion exchange chromatography step, optionally, a cation exchange chromatography step or an anion exchange chromatography step.
E35. The method of any one of the preceding embodiments, wherein each antibody of the antibody composition is an IgG.
E36. The method of embodiment 35, wherein each antibody of the antibody composition is an IgG₁.
E37. The method of any one of the preceding embodiments, wherein each antibody of the antibody composition binds to a tumor-associated antigen.
E38. The method of embodiment 37, wherein the tumor-associated antigen comprises the amino acid sequence of SEQ ID NO. 3.
E39. The method of any one of the preceding embodiments, wherein each antibody of the antibody composition is an anti-CD20 antibody.
E40. The method of any one of the preceding embodiments, wherein each antibody of the antibody composition comprises:
   i. a light chain (LC) CDR1 comprising an amino acid sequence of SEQ ID NO: 4 or an amino acid sequence which is at least 90% identical to SEQ ID NO: 4 or a variant amino acid sequence of SEQ ID NO: 4 with 1 or 2 amino acid substitutions,
   ii. a LC CDR2 comprising an amino acid sequence of SEQ ID NO: 5 or an amino acid sequence which is at least 90% identical to SEQ ID NO: 5 or a variant amino acid sequence of SEQ ID NO: 5 with 1 or 2 amino acid substitutions,
   iii. a LC CDR3 comprising an amino acid sequence of SEQ ID NO: 6 or an amino acid sequence which is at least 90% identical to SEQ ID NO: 6 or a variant amino acid sequence of SEQ ID NO: 6 with 1 or 2 amino acid substitutions,
   iv. a heavy chain (HC) CDR1 comprising an amino acid sequence of SEQ ID NO: 7 or an amino acid sequence which is at least 90% identical to SEQ ID NO: 7 or a variant amino acid sequence of SEQ ID NO: 7 with 1 or 2 amino acid substitutions;
   v. a HC CDR2 comprising an amino acid sequence of SEQ ID NO: 8 or an amino acid sequence which is at least 90% identical to SEQ ID NO: 8 or a variant amino acid sequence of SEQ ID NO: 8 with 1 or 2 amino acid substitutions; and
   vi. a HC CDR3 comprising an amino acid sequence of SEQ ID NO: 9 or an amino acid sequence which is at least 90% identical to SEQ ID NO: 9 or a variant amino acid sequence of SEQ ID NO: 9 with 1 or 2 amino acid substitutions.
E41. The method of any one of the preceding embodiments, wherein each antibody of the antibody composition comprises a LC variable region comprising an amino acid sequence of SEQ ID NO: 10, an amino acid sequence which is at least 90% identical to SEQ ID NO: 10, or a variant amino acid sequence of SEQ ID NO: 10 with 1 to 10 amino acid substitutions.
E42. The method of any one of the preceding embodiments, wherein each antibody of the antibody composition comprises a HC variable region comprising an amino acid sequence of SEQ ID NO: 11, an amino acid sequence which is at least 90% identical to SEQ ID NO: 11, or a variant amino acid sequence of SEQ ID NO: 11 with 1 to 10 amino acid substitutions.
E43. The method of any one of the preceding embodiments, wherein each antibody of the antibody composition comprises a light chain comprising an amino acid sequence of SEQ ID NO: 12, an amino acid sequence which is at least 90% identical to SEQ ID NO: 12, or a variant amino acid sequence of SEQ ID NO: 12 with 1 to 10 amino acid substitutions.
E44. The method of any one of the preceding embodiments, wherein each antibody of the antibody composition comprises a heavy chain comprising an amino acid sequence of SEQ ID NO: 13, an amino acid sequence which is at least 90% identical to SEQ ID NO: 13, or a variant amino acid sequence of SEQ ID NO: 13 with 1 to 10 amino acid substitutions.
E45. A method of producing an antibody composition within a target % ADCC range said method comprising:
   i. measuring the % ADCC of a series of samples comprising varying glycoforms of an antibody,
   ii. determining the % total afucosylated (TAF) glycans for each sample of the series,
   iii. determining a linear equation of a best fit line of a graph which plots for each sample of the series the % ADCC as measured in step (i) as a function of the % TAF glycans as determined in step (ii),
   iv. determining the % TAF for an antibody composition and then calculating a % ADCC using the linear equation of step (iii), and
   v. selecting the antibody composition for one or more downstream processing steps when the % ADCC calculated in step (iv) is within a target % ADCC range.
E46. A method of producing an antibody composition within a target % total afucosylated (TAF) range said method comprising:
   i. measuring the % ADCC of a series of samples comprising varying glycoforms of an antibody,
   ii. determining the % total afucosylated (TAF) glycans for each sample of the series,
   iii. determining a linear equation of a best fit line of a graph which plots for each sample of the series the % ADCC as measured in step (i) as a function of the % TAF glycans as determined in step (ii),
   iv. determining the % ADCC for an antibody composition and then calculating a % TAF using the linear equation of step (iii), and
   v. selecting the antibody composition for one or more downstream processing steps when the % TAF calculated in step (iv) is within a target % TAF range.
E47. A method of determining % antibody dependent cellular cytotoxicity (ADCC) of an antibody composition, said method comprising:
   i. determining the % total afucosylated (TAF) glycans of an antibody composition;
   ii. calculating the % ADCC of the antibody composition based on the % TAF using Equation A:

Y=2.6+24.1*X [Equation A],
wherein Y is the % ADCC and X is the % TAF glycans determined in step (i),
E48. A method of determining % antibody dependent cellular cytotoxicity (ADCC) of an antibody composition, said method comprising
   i. determining the % high mannose glycans and the % afucosylated glycans of an antibody composition,
   ii. calculating the % ADCC of the antibody composition based on the % high mannose glycans and the % afucosylated glycans using Equation B:

Y=(0.24+27*HM+22.1*AF) [Equation B],

wherein Y is the % ADCC, HM is the % high mannose glycans determined in step (i), and AF is the % afucosylated glycans determined in step (i), and E49. The method of embodiment 47 or 48, further comprising selecting the antibody composition for one or more downstream processing steps when Y is within a target % ADCC range.
E50. A method of producing an antibody composition within a target % TAF range said method comprising the following steps:
   i. generating a linear equation of a best fit graph by plotting the % ADCC and % TAF glycans of a series of at least 5 reference antibody compositions produced under cell culture conditions, each reference antibody composition having the same amino acid sequence as the antibody composition;
   ii. selecting a target % TAF glycan range based on the linear equation generated in step (i) and desired % ADCC activity;
   iii. culturing the antibody composition under cell culture conditions;
   iv. purifying the antibody composition;
   v. sampling the antibody composition to determine the % TAF; and
   vi. determining whether the % TAF of the antibody composition is within the target % TAF range of step (ii).
E51. The method of embodiment 50, further comprising selecting the antibody composition for one or more downstream processing steps when the % TAF calculated in step (v) is within the target % TAF range.

The following examples are given merely to illustrate the present invention and not in any way to limit its scope.

### EXAMPLES

### Example 1

This example describes an exemplary method of determining an N-linked glycosylation profile for an antibody.

The purpose of this analytical method is to determine the N-linked glycosylation profile of a particular antibody in samples comprising the antibody by hydrophilic interaction chromatography. This glycan map method is a quantitative purity analysis of the N-linked glycan distribution of the antibody.

Briefly, N-linked glycans are enzymatically released using N-glycosidase F (PNGase F) and the terminal N-acetylglucosamine (GlcNAc) is derivatized with fluorophore. The labeled glycans are then separated using a hydrophilic interaction column (HILIC). The analytical method consists of these steps: (1) release and label N-linked glycans from reference and test samples using PNGase F and a fluorophore that can specifically derivatize free glycan, (2) load samples within the validated linear range onto a HILIC column, the labeled N-linked glycans are separated using a gradient of decreasing organic solvent, and (3) monitor elution of glycan species with fluorescence detector.

The standard and test samples are prepared by carrying out the following steps: (1) dilute samples and controls with water, (2) add PNGase F and incubate the samples and controls to release N-linked glycans, (3) mix with fluorophore labeling solution using a fluorophore such as 2-aminobenzoic acid. Vortex and incubate the samples and controls, (4) centrifuge down to pellet protein and remove supernatant, and (5) dry and reconstitute labeled glycans in the injection solution.

The reagents used in this assay are a Mobile Phase A (100 mM ammonium formate, target pH 3.0) and a Mobile Phase B (acetonitrile). The equipment used to perform steps of the method have the following capabilities:

Equipment capabilities:

HPLC system
Fluorescence detector set to appropriate excitation/emission
wavelength optimized to labeling fluorophore
Data collection system
Temperature-controlled autosampler
Hydrophilic interaction column

The instrument settings for HPLC using a hydrophilic interaction analytical 1.7 μm column, 2.1 mm ID×150 mm are provided below:

| | | |
|---|---|---|
| Target sample load | 2 | μL |
| Column heater set point | 35° | C. |
| Auto-sampler set point | 10° | C. |

| | |
|---|---|
| Detection | Excitation 360 nm |
| | Emission 425 nm |

The recommended gradient is provided below:

| Time | Flow Rate | Mobile Phase A | Mobile Phase B |
|---|---|---|---|
| (minutes) | (mL/minute) | (%) | (%) |
| 0.0 | 0.25 | 22.0 | 78.0 |
| 111.2 | 0.25 | 40.1 | 59.9 |
| 117.9 | 0.20 | 90.0 | 10.0 |
| 124.5 | 0.20 | 90.0 | 10.0 |
| 129.1 | 0.25 | 22.0 | 78.0 |
| 155 | 0.25 | 22.0 | 78.0 |

The system suitability are provided below:

System Suitability Criteria
% RSD of the total integrated peak area of all reference standard injections
must be ≤ 10%
% RSD for A2G0F peak retention time for all reference standards must
be ≤ 2%
% RSD of A2G0F, M5, and A2G2F peak percent area of all reference
standard injections must be ≤ 5%
Signal to noise for A2G0F peak of all reference standard injections must
be ≥ limit of quantitation threshold
Sample Acceptance Criteria
Total peak area based on the total integrated peak area of the sample must
be 50% to 150% of the average total peak area of the reference standard
injections
The retention time difference between the sample and the average of all
reference standard injections for A2G0F peak must be ≤ 0.5 minutes

The results are reported as follows:

Report area % for total afucosylation, afucosylation,
high mannose, and galactosylation
Calculations examples:

% Total afucosylation (% total Afuc) = % A1G0 +
% A2G0 + % A2G1(a) + % A2G1(b) + % A2G2 +
% A1G1M5 + % M5 + % M6 + % M7
% High mannose (% HM) = % M5 + % M6 + % M7
% Afucosylation (% Afuc) = % A1G0 + % A2G0 +
% A2G1(a) + % A2G1(b) + % A2G2 + % A1G1M5
% Galactosylation (% Gal) = % A2G1(a) + % A1G1F +
% A2G1(b) + % A2G1F(a) + % A2G1F(b) + % A2G2 +
% A3G1F + % A1G1M5 + % A2G2F + % A1G1M5F +
% A3G2F(a) + % A3G2F(b) + % A2G2S1F(a) +
% A2G2S1F(b)

A representative glycan map chromatogram is shown in FIG. 2A (full scale view) and FIG. 2B (expanded scale view).

### Example 2

This example describes an exemplary assay to assess ADCC activity of an anti-CD20 antibody using engineered effector cells.

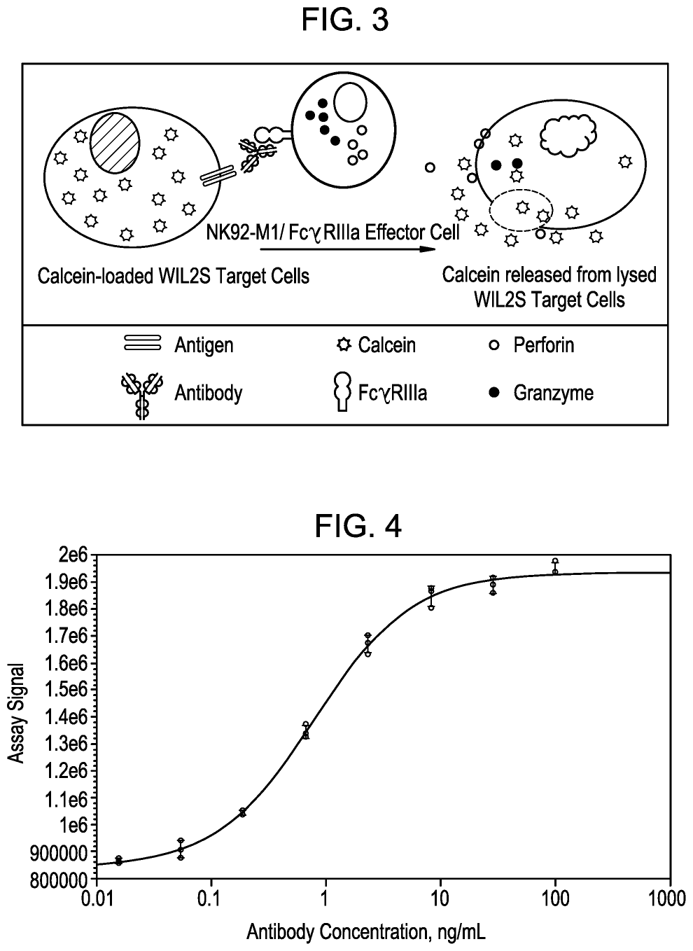

The purpose of this analytical method is to determine the Antibody Dependent Cellular Cytotoxicity (ADCC) level of an antibody, expressed as a %. This ADCC bioassay is a quantitative cell-based assay that measures the ability of an anti-CD20 antibody to mediate cell cytotoxicity in a dose-dependent manner in CD20-expressing B-lymphocytes by binding to CD20 antigen on WIL2-S (human B-lymphocyte) and engaging FcγRIIIA (158V) receptors on NK92-M1 effector cells via the antibody Fc domain. This leads to the activation of the effector cell and destruction of the tumor cell via exocytosis of the cytolytic granule complex perforin/granzyme. A schematic of the ADCC assay is provided in FIG. 3 and a representative dose-response curve for the ADCC assay is shown in FIG. 4. In FIG. 4, each dose point is a mean±standard deviation of 3 replicates and the assay signal=fluorescence.

The method consists of these steps

| Step | Action |
|---|---|
| 1 | Label WIL2-S target cells with Calcein-AM |
| 2 | Add labelled WIL2-S cell suspension to plate |
| 3 | Add reference standard, control, and test sample dilutions to |
| | plate |
| 4 | Incubate at 37° C. in a humidified incubator for 45 to 50 minutes |
| 5 | Add NK92-M1 effector cells at 25:1 (Effector:Target) ratio |
| 6 | Incubate at 37° C. in a humidified incubator for 55 to 65 minutes |
| 7 | Remove plates from incubator and centrifuge at 750-850 RPM |
| | for 5 minutes |
| 8 | Filter 100 uL supernatant into assay plate |
| 9 | Read assay plates using a plate reader and analyze data |

The standard and test samples are prepared by diluting the reference standard, assay control, and sample to cover the validated dose range.

The reagents used in this assay include the following and the composition of each is provided:

| Reagent | Composition |
|---|---|
| Growth medium for WIL2-S | RPMI 1640 medium |
| cells | |
| Fetal bovine serum (FBS) | 10% FBS in growth medium |
| PSG | 1X Penicillin-Streptomycin-Glutamine |
| Human B-lymphoblastoid | Cultured |
| cell line (WIL2-S) | |
| Growth medium for NK92- | MEM alpha |
| M1 cells | |
| Horse Serum | 8% Horse Serum in growth medium |
| Fetal bovine serum (FBS) | 8% FBS in growth medium |
| Myoinositol | Commercial |
| Folic Acid | Commercial |
| β - Mercaptoethanol | Commercial |
| Blasticidin | Commercial |
| NK92-M1 cells | Cultured |
| PSG | 1X Penicillin-Streptomycin-Glutamine |
| Calcein-AM ® | Commercial fluorescent cell labeling |
| | reagent |

Certain steps of the method require a microplate reader with fluorescence capacity.

The system suitability are as follows:

Data analysis is performed using a 4 parameter logistic (4PL) curve
fit for each three plate set-up The analysis is performed using
2 model curve fits of the dose response data. The full model is
fit to determine if the test sample and the reference standard
are parallel (the curve shapes are sufficiently similar).

Assay Acceptance Criteria
Reference standard curve R² ≥ 0.95
Reference standard max-to-min ratio must be greater than or equal to 2
Relative potency of the control must be within 80 and 120%
The % CV of all replicates of all concentration doses of the Reference
Standard must be < 15%
Control sample must pass all sample acceptance criteria
Sample Acceptance Criteria
Sample curve R² ≥ 0.95
The F-prob value of the test sample must be ≥ 0.01
The % CV of all replicates of all concentration doses of the test
sample(s) must be < 15%
Three sample determinations must fall within ± 25% of the arithmetic
mean

The results are reported as % relative ADCC.

### Example 3

This example describes a study which led to establishing a model relating ADCC to glycan levels.

Representative samples (N=41) of the anti-CD20 antibody made in small-scale bioreactors were assessed for levels of the following glycoforms: high mannose, afucosylation, and galactosylation, using the exemplary method described in Example 1. % of total afucosylation (% TAF) is the sum of % High Mannose and % Afucosylation. ADCC levels for each representative sample of the anti-CD20 antibody was determined by the assay described in Example 2. The results are provided in Table 1.

TABLE 1

| | | | Total | | |
|---|---|---|---|---|---|
| | High | Afucosyl- | Afucosyl- | Galactosyl- | |
| | Mannose | ation | ation | ation | ADCC |
| Sample | (%) | (%) | (%) | (%) | (%) |
| Sample A | 1.656 | 2.470 | 4.126 | 60.017 | 110 |
| Sample B | 3.165 | 2.884 | 6.049 | 54.611 | 169 |
| Sample C | 2.057 | 1.326 | 3.383 | 58.090 | 86 |
| Sample D | 2.054 | 2.510 | 4.564 | 55.992 | 105 |
| Sample E | 2.028 | 2.383 | 4.411 | 57.525 | 102 |
| Sample F | 2.134 | 2.144 | 4.278 | 55.973 | 106 |
| Sample G | 2.865 | 2.698 | 5.563 | 56.957 | 143 |
| Sample H | 2.960 | 3.022 | 5.982 | 58.036 | 143 |
| Sample I | 2.185 | 1.596 | 3.781 | 56.888 | 92 |
| Sample J | 1.574 | 1.349 | 2.923 | 58.433 | 75 |
| Sample K | 1.941 | 2.059 | 4.000 | 53.868 | 94 |
| Sample L | 1.610 | 2.478 | 4.088 | 60.385 | 104 |
| Sample M | 1.496 | 2.034 | 3.530 | 60.419 | 81 |
| Sample N | 1.995 | 1.352 | 3.348 | 58.638 | 83 |
| Sample O | 2.121 | 1.842 | 3.962 | 54.106 | 102 |
| Sample P | 2.187 | 1.794 | 3.981 | 51.431 | 84 |
| Sample Q | 1.527 | 1.144 | 2.671 | 58.953 | 61 |
| Sample R | 1.749 | 1.914 | 3.663 | 56.991 | 84 |
| Sample S | 2.244 | 2.035 | 4.279 | 52.025 | 95 |
| Sample T | 1.814 | 1.689 | 3.504 | 54.068 | 76 |
| Sample U | 1.677 | 0.996 | 2.672 | 58.941 | 64 |
| Sample V | 1.573 | 1.221 | 2.794 | 59.691 | 67 |
| Sample W | 2.073 | 1.900 | 3.973 | 53.687 | 92 |
| Sample X | 1.615 | 1.181 | 2.796 | 57.400 | 67 |
| Sample Y | 1.597 | 1.028 | 2.626 | 58.113 | 67 |
| Sample Z | 2.551 | 2.380 | 4.931 | 57.280 | 122 |
| Sample AA | 1.940 | 1.216 | 3.156 | 55.795 | 88 |
| Sample BB | 1.877 | 1.307 | 3.184 | 54.237 | 86 |
| Sample CC | 1.737 | 1.260 | 2.997 | 59.487 | 82 |
| Sample DD | 2.085 | 2.158 | 4.243 | 58.090 | 108 |
| Sample EE | 2.102 | 2.034 | 4.136 | 54.150 | 97 |
| Sample FF | 1.363 | 1.045 | 2.408 | 62.542 | 75 |
| Sample GG | 1.838 | 1.641 | 3.479 | 51.823 | 80 |
| Sample HH | 2.133 | 2.044 | 4.178 | 57.188 | 92 |
| Sample II | 1.907 | 1.876 | 3.783 | 55.337 | 95 |
| Sample JJ | 2.030 | 2.104 | 4.134 | 54.992 | 105 |
| Sample KK | 2.235 | 1.318 | 3.554 | 52.830 | 85 |
| Sample LL | 2.016 | 1.144 | 3.161 | 54.867 | 75 |
| Sample MM | 1.844 | 1.294 | 3.138 | 55.187 | 87 |
| Sample NN | 1.835 | 1.764 | 3.599 | 56.959 | 101 |
| Sample OO | 1.529 | 1.184 | 2.714 | 59.766 | 78 |

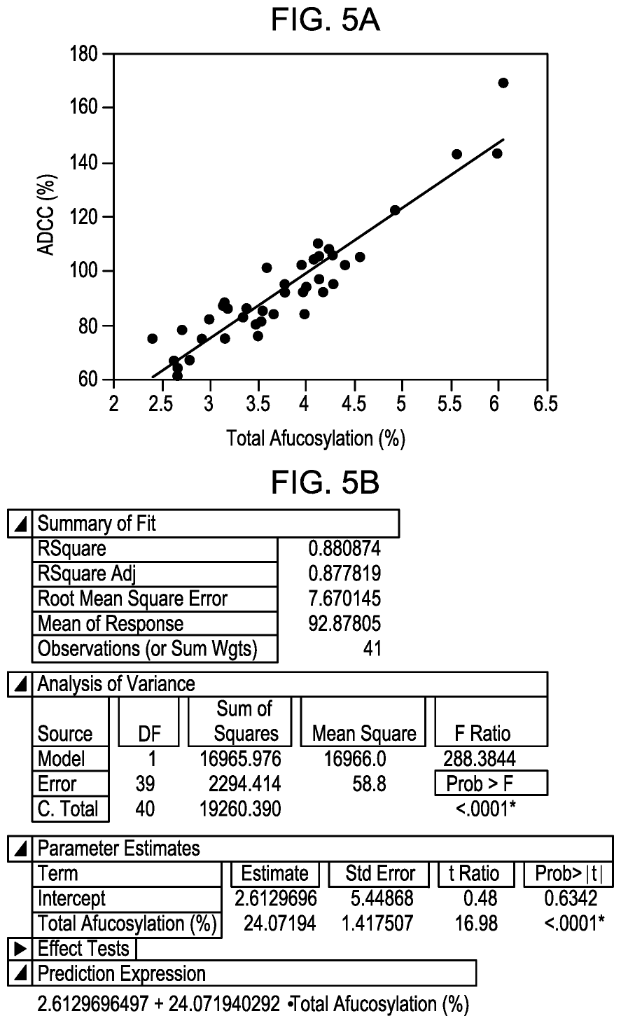

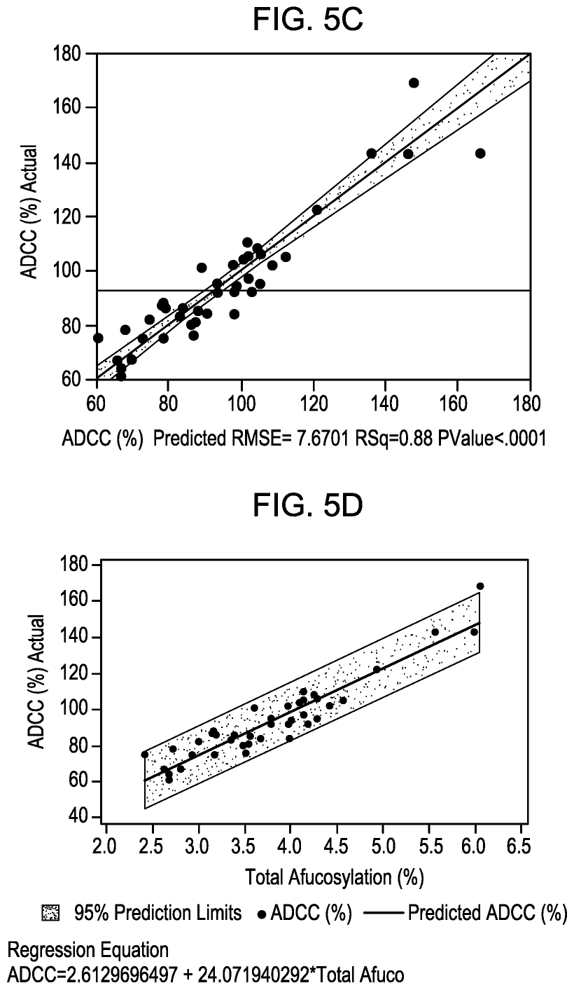

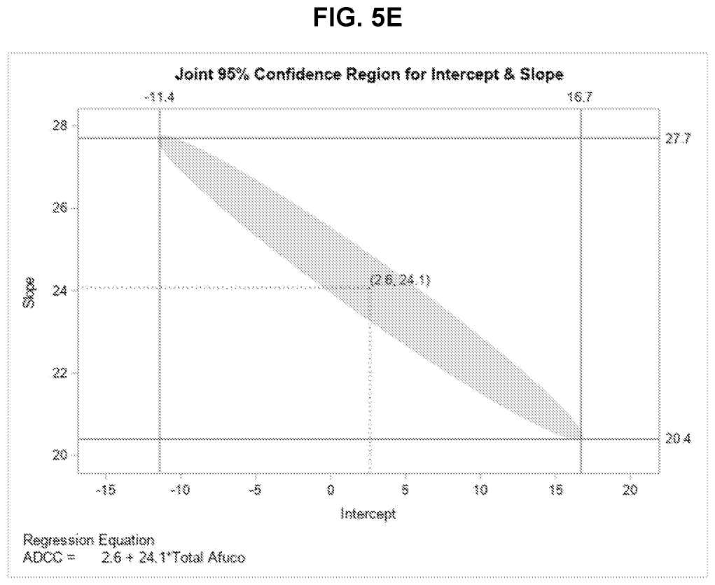

The data of Table 1 were analyzed using the JMP suite of computer programs for statistical analysis (SAS Institute, Cary, N.C.). A regression plot of the data is provided in FIG. 5A. The best fit line of the plotted data is shown in this figure and may be described by the following linear equation, Equation 1:

% ADCC=2.6129696497+24.071940292*% TAF [Equation 1].

Additional statistical parameters are provided in FIG. 5B. As shown in this figure, the significance of the association between ADCC and TAF was demonstrated by the r² value (r²=0.88) and p value (p<0.0001).

Using Equation 1 and the TAF values of Table 1, a Predicted % ADCC value was calculated for each sample in Table 1. The Actual ADCC % (listed in Table 1) was plotted against the Predicted % ADCC in FIG. 5C. The results confirmed that there is a direct correlation between total afucosylation and ADCC with higher level of total afucosylation resulting in higher ADCC activity.

FIG. 5D is the same graph as FIG. 5A but with a graphical depiction of the 95% confidence interval (shown by light blue area). As shown in FIG. 5D, most data points fell within the 95% confidence interval. FIG. 5E provides a graph of the 95% confidence region for both the y-intercept and slope of Equation 1.

The data of Table 1 using the individual components of TAF (Afucosylation (AF) and high mannose (HM)) also were analyzed using the JMP suite and showed a similar correlation to ADCC.

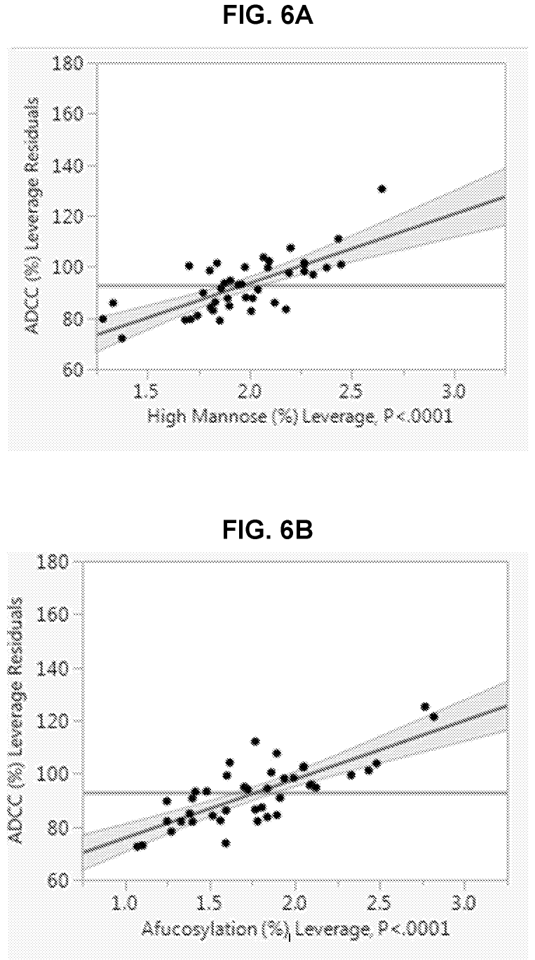

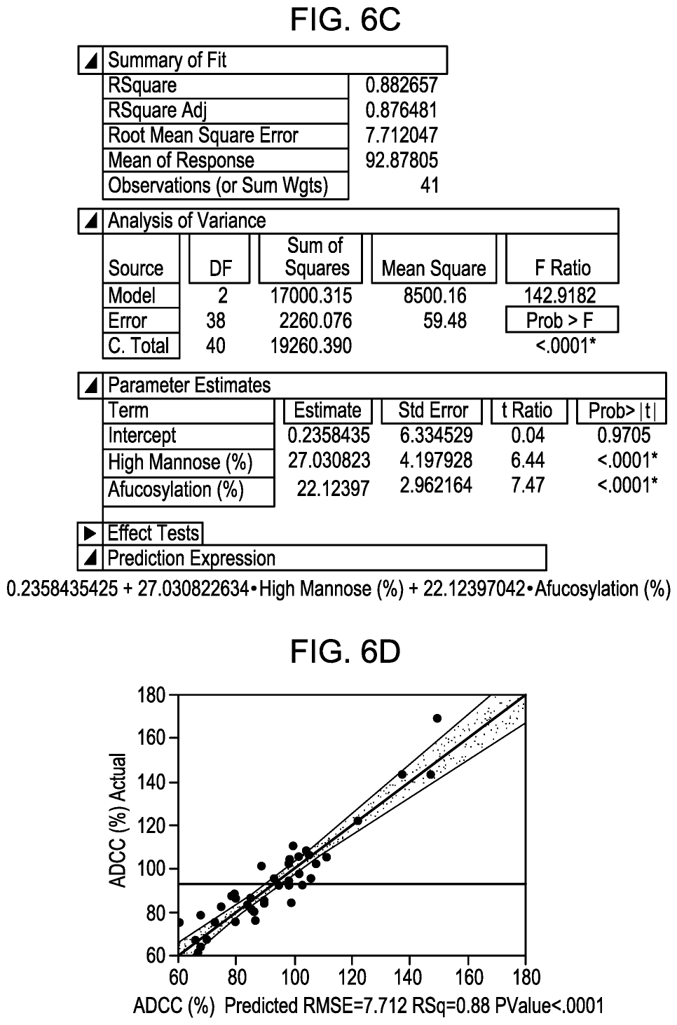

FIGS. 6A and 6B provide a regression plot for these data on High Mannose and Afucosylation. The best fit line of the plotted data is shown in each of FIGS. 6A and 6B and may be described by the following linear equation, Equation 2:

% ADCC=0.2358435425+27.030822634*% HM+22.12397042*% AF [Equation 2]

Additional statistical parameters are provided in FIG. 6C. As shown in this figure, the significance of the association between ADCC and TAF was demonstrated by the r² values (r²=0.88) and p values (p<0.0001).

Using Equation 2 and the high mannose and afucosylation values of Table 1, a Predicted % ADCC value was calculated for each sample in Table 1. The Actual ADCC % (listed in Table 1) was plotted against the Predicted % ADCC in FIG. 6D. The results confirmed that there is a direct correlation between afucosylated glycans, high mannose, and ADCC, with higher levels of afucosylated glycans and high mannose resulting in higher ADCC activity. Afucosylated glycans and high mannose had a similar contribution to ADCC activity.

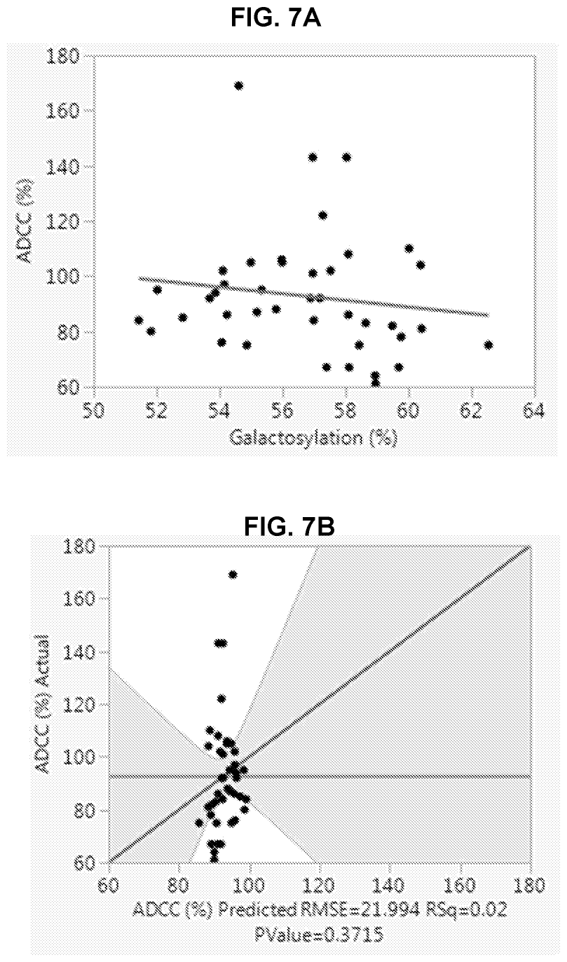

The association between ADCC and HM and AF (or TAF) was specific to these glycans, as galactosylation did not demonstrate a statistically significant association. FIG. 7A is a regression plot between ADCC and galactosylation levels. The lack of statistical significance was demonstrated by the r² values (r²=0.02) and p value (p<0.3715). FIG. 7B is a graph of the Actual ADCC % (listed in Table 1) plotted as a function of the predicted ADCC. As shown in these figures, only a very weak association was observed between ADCC and galactosylation.

TAF was confirmed by statistical analysis to have the most significant contribution to ADCC activity. The association of TAF levels to ADCC activity levels was very different from the relationship between % ADCC and other glycans.

### Example 4

This example describes a study validating the model relating ADCC to TAF.

The model described in Example 3 associating ADCC to TAF was validated using large-scale manufacturing samples of the same antibody of the large-scale bioreactor samples in Table 1. Each large-scale sample (N=13) was characterized for TAF levels by measuring the high mannose and afucosylation levels following the method described in Example 1 and then summing the two % to obtain % TAF levels. The experimental ADCC level for each large-scale sample was determined by carrying out the assay described in Example 2, repeating twice to get 3 values per sample and then recording the average of the 3 values. A predicted ADCC was calculated by using Equation 1. The results are provided in Table 2 below.

TABLE 2

| | Total | Experimental | Predicted |
|---|---|---|---|
| Sample # | Afucosylation | ADCC* | ADCC |
| Sample 1 | 3.7 | 93 | 92 |
| Sample 2 | 3.3 | 78 | 82 |
| Sample 3 | 3.7 | 92 | 92 |
| Sample 4 | 3.5 | 93 | 87 |
| Sample 5 | 3.2 | 83 | 79 |
| Sample 6 | 3.0 | 88 | 76 |
| Sample 7 | 3.6 | 88 | 89 |
| Sample 8 | 3.2 | 79 | 80 |
| Sample 9 | 3.8 | 95 | 93 |
| Sample 10 | 3.7 | 90 | 92 |
| Sample 11 | 3.9 | 92 | 97 |
| Sample 12 | 3.8 | 93 | 94 |
| Sample 13 | 3.8 | 87 | 94 |

*average of three values

As shown by the data in Table 2, the predicted ADCC results generated by the Equation 1 is strongly aligned with the reported experimental results. Therefore, a reliable and precise model associating with ADCC and TAF was established.

### Example 5

This example describes a novel glycan model reveals a basis for predicting ADCC for an anti-CD20 antibody.

An anti-CD20 antibody is being developed as a biosimilar to Rituximab. It is a recombinant chimeric mouse/human IgG1 monoclonal antibody that specifically binds to the CD20 antigen expressed on B cells and promotes B cell killing through multiple mechanisms, with ADCC being one of the important mechanism of actions. It is well-established that the absence of core fucose leads to increased ADCC activity while galactosylation and high mannose may also play a role. A systematic assessment of the contribution of N-glycans to the anti-CD20 antibody ADCC activity was performed via glyco engineering studies and confirmed that there is a direct correlation between afucosylated glycans, high mannose, and ADCC, with higher levels of afucosylated glycans and high mannose resulting in higher ADCC activity. However, the glycan profile of samples produced via glyco-engineering may not be fully representative of the glycan attribute range of anti-CD20 antibody, a statistical assessment of small scale bioreactor datasets of anti-CD20 antibody was performed to establish a representative glycan ADCC model by capturing the full range in the anti-CD20 antibody manufacturing process. Using this approach, it reveals that afucosylation and high mannose showed similar correlation to ADCC. A novel methodology was applied to the glycan model that Total Afucosylation (sum of Afucosylation and high mannose) was used to predict anti-CD20 antibody ADCC. A prediction expression (ADCC=2.6+24.1×Total Afucosylation) was established and validated using large scale manufacturing data. The predicted ADCC result generated by the expression is strongly aligned with the reported ADCC assay results. Therefore, the correlation of total afucosylation and ADCC was established as the glycan-ADCC model and enable process to monitor ADCC using glycan measurement as an orthogonal approach.

The outcome of this work identified a basis for the glycan correlation with ADCC results in functional assays between anti-CD20 antibody and the orthogonal method (HPLC glycan method). The data enabled Amgen to proceed with an attribute focused development approach and an identified mechanism to account for the results and provide novel attribute analysis for the market application.

Approaches used included HPLC, ADCC assay and cross functional collaboration

### Example 6

This example demonstrates a study which led to establishing a model relating ADCC to glycan levels for a second antibody.

Example 3 describes a study which led to establishing a model relating ADCC to glycan levels for an IgG1 which binds to CD20. This study evaluates the relationship between ADCC and glycan levels for a chimeric, monoclonal IgG1 kappa antibody composed of human constant and murine variable regions and binds to the TNFα antigen.

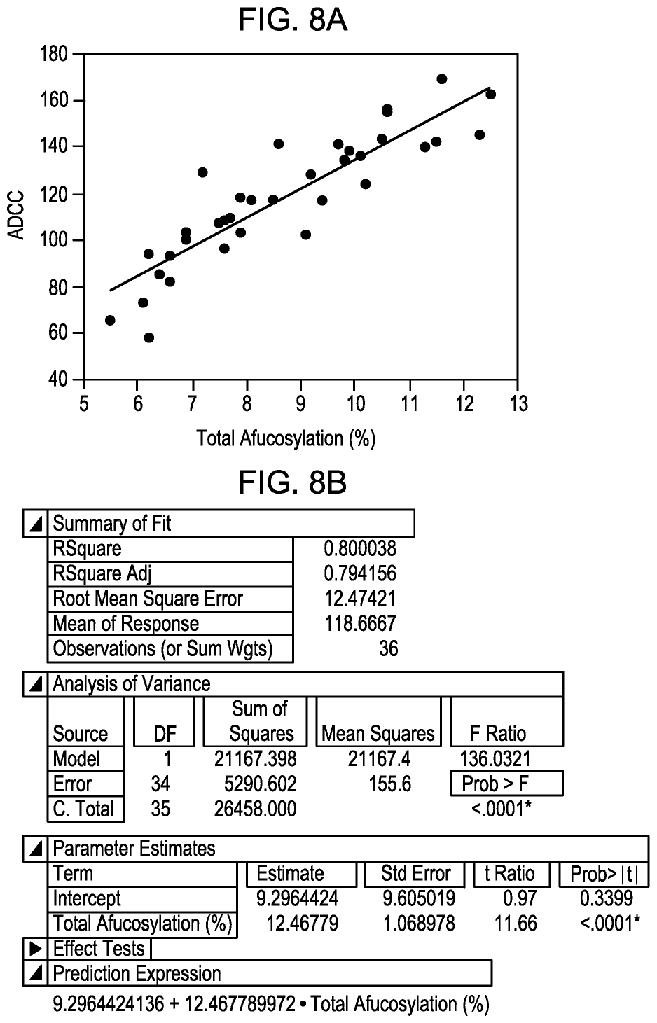

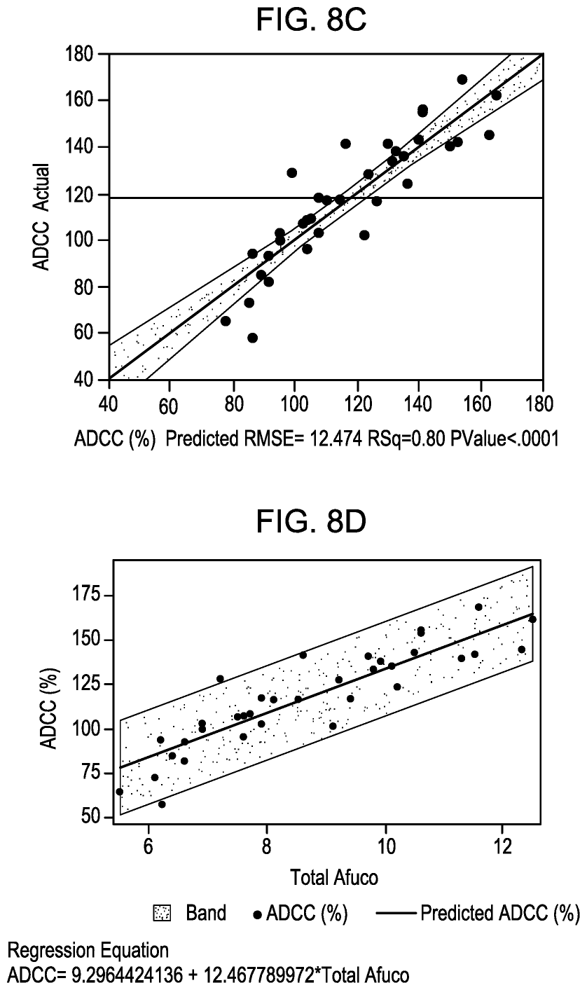

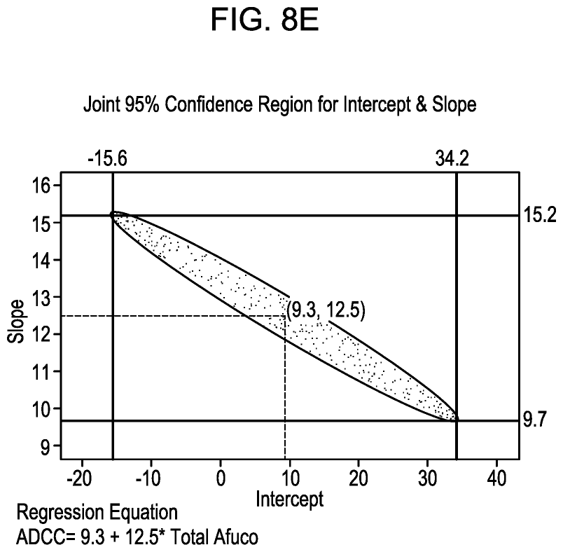

Representative samples of the second antibody (anti-TNFα antibody) made in small-scale bioreactors were assessed for levels of the following glycoforms: high mannose, and afucosylation, using the exemplary method described in Example 1. Percentage of total afucosylation (% TAF) is the sum of % High Mannose and % Afucosylation. ADCC levels for each representative sample of the anti-TNFα antibody was determined by the assay described in Example 2. The data were analyzed using the JMP suite of computer programs for statistical analysis (SAS Institute, Cary, N.C.). A regression plot of the data is provided in FIG. 8A. The best fit line of the plotted data is shown in this figure and may be described by the following linear equation, Equation 3:

% ADCC=9.3+12.47*% TAF [Equation 3].

Additional statistical parameters are provided in FIG. 8B. As shown in this figure, the significance of the association between ADCC and TAF was demonstrated by the r² value (r²=0.80) and p value (p<0.0001).

Using Equation 3 and the measured TAF values, a Predicted % ADCC value was calculated for each sample. The Actual ADCC % (measured as described in Example 2) was plotted against the Predicted % ADCC in FIG. 8C. The results confirmed that there is a direct correlation between total afucosylation and ADCC with higher level of total afucosylation resulting in higher ADCC activity.

FIG. 8D is the same graph as FIG. 8A but with a graphical depiction of the 95% confidence interval (shown by grey shaded area). As shown in FIG. 8D, most data points fell within the 95% confidence interval. FIG. 8E provides a graph of the 95% confidence region for both the y-intercept and slope of Equation 3.

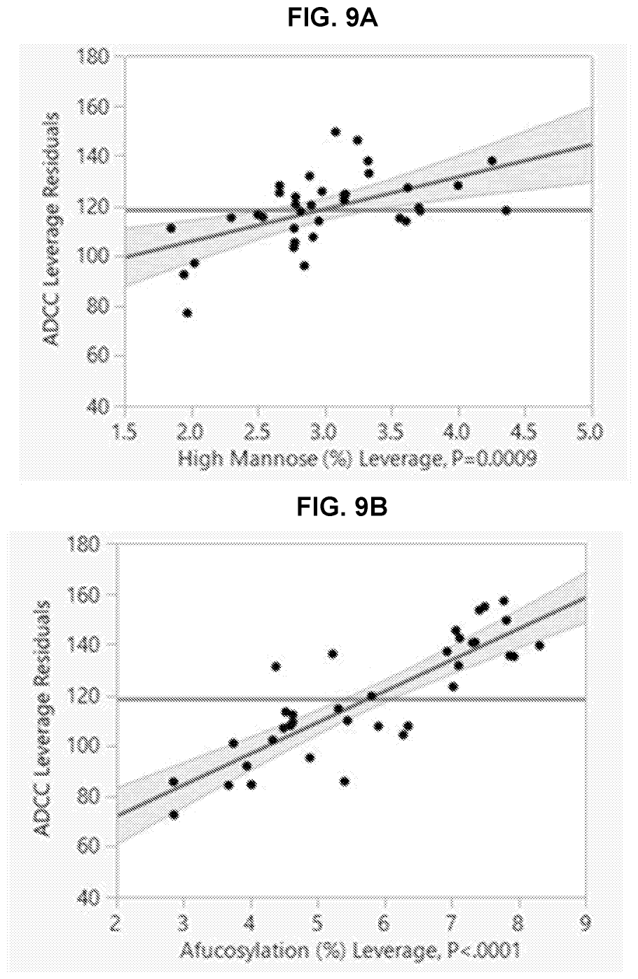

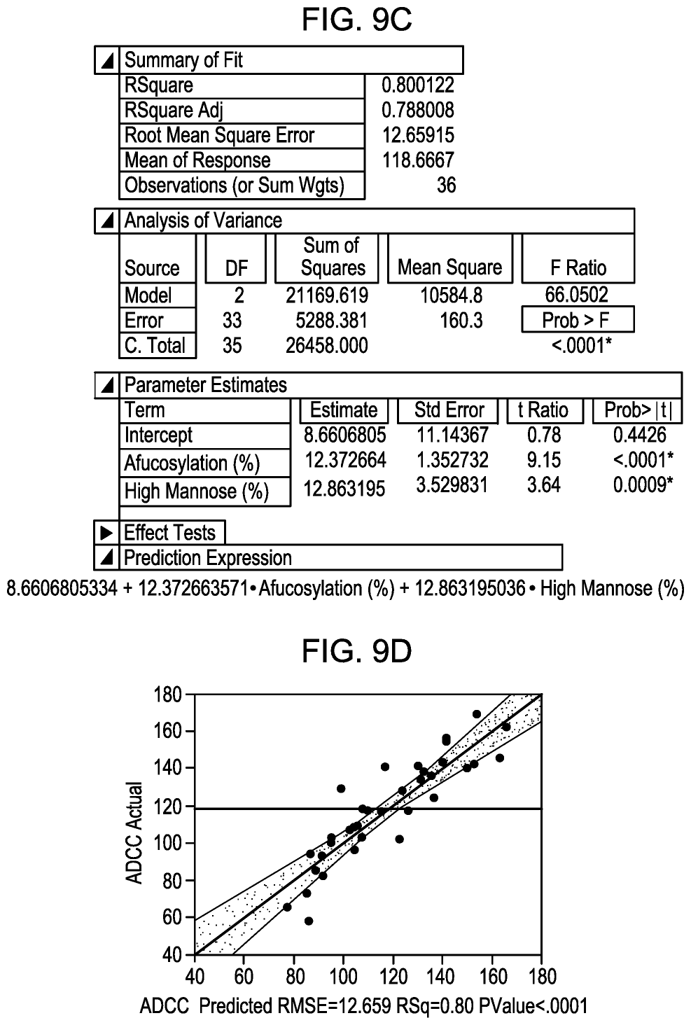

The data using the individual components of TAF (Afucosylation (AF) and high mannose (HM)) also were analyzed using the JMP suite and showed a similar correlation to ADCC. FIGS. 9A and 9B provide a regression plot for these data on High Mannose and Afucosylation, respectively. The best fit line of the plotted data is shown in each of FIGS. 9A and 9B and may be described by the following linear equation, Equation 4:

% ADCC=8.66+12.86*% HM+12.37*% AF [Equation 4]

Additional statistical parameters are provided in FIG. 9C. As shown in this figure, the significance of the association between ADCC and TAF was demonstrated by the r² values (r²=0.8) and p values (p<0.0001).

Using Equation 4 and the measured high mannose and afucosylation values, a Predicted % ADCC value was calculated for each sample. The Actual ADCC % (measured as described in Example 2) was plotted against the Predicted % ADCC in FIG. 9D. The results confirmed that there is a direct correlation between afucosylated glycans, high mannose, and ADCC, with higher levels of afucosylated glycans and high mannose resulting in higher ADCC activity. Afucosylated glycans and high mannose had a similar contribution to ADCC activity.

This example demonstrated that, for the second antibody (anti-TNFα antibody), TAF was confirmed by statistical analysis to have a highly significant contribution to ADCC activity.

### Example 7

This example demonstrates a second set of models relating ADCC to TAF, HM and/or AF glycans.

Each of Examples 3 and 6 establishes a linear regression model relating ADCC to TAF glycan content or ADCC to HM and AF glycan content for two antibodies: an anti-CD20 antibody and an anti-TNFalpha antibody. The models are mathematically described in Equations 1-4. For each of these equations, the importance of the y-intercept was evaluated by analyzing the p-value of the y-intercepts of each equation. Table 3 provides the p-value for the y-intercepts for each of Equations 1-4.

TABLE 3

| Equation | p-value |
|---|---|
| 1 | 0.6331 |
| 2 | 0.9705 |
| 3 | 0.3399 |
| 4 | 0.4426 |

As each of the p-values were greater than 0.05, each y-intercept of Equations 1-4 were considered as close to zero and could be dropped from the equation.

Given the above, the measured ADCC data and measured glycan data were re-fitted to a “no y-intercept model” and the statistical significance of these models were evaluated. Table 4 lists the equations of the no y-intercept model describing the relationship between ADCC and TAF glycans or ADCC and HM and AF glycans for the two antibodies.

TABLE 4

| | | | Statistical |
|---|---|---|---|
| Antibody | Eq. # | No y-intercept model | paramters |
| Anti-CD20 | 5 | ADCC = 24.73579 * TAF | r² = 0.9938 |
| | | | p-value <0.0001 |
| | 6 | ADCC = 27.14941 * HM + | r² = 0.9939 |
| | | 22.12018 * AF | p-value <0.0001 |
| Anti-TNFα | 7 | ADCC = 13.47790 * TAF | r² = 0.9898 |
| | | | p-value <0.0001 |
| | 8 | ADCC = 14.84841 * HM + | r² = 0.9899 |
| | | 12.78827 * AF | p-value <0.0001 |

As shown in Table 4, the no y-intercept models are statistically significant and represent for alternative models that correlate ADCC to TAF glycan content or ADCC to HM and AF glycan content.

Table 5 provides the slopes for each of the linear regression models and the no y-intercept models.

TABLE 5

| | | Slope of Linear | | Slope of No y- | |
|---|---|---|---|---|---|
| | | Regression Model for | | Intercept Model for | |
| | | indicated glycan | | indicated glycan | |
| Anti-CD20 | TAF | 24.07070 | (0.93857) | 24.73579 | (0.99690) |
| | HM | 27.03082 | (0.47699) | 27.14941 | (0.57227) |
| | AF | 22.12397 | (0.55327) | 22.12018 | (0.43068) |
| Anti- | TAF | 12.46779 | (0.89445) | 13.47790 | (0.99489) |
| TNFalpha | HM | 12.86320 | (0.29957) | 14.84841 | (0.37364) |
| | AF | 12.37266 | (0.75188) | 12.78827 | (0.63092) |

Standardized estimates are provided in ( )s.

As shown in Table 5, the two models are in high agreement with one another. The x-intercepts for TAF (24.07070 vs. 24.73579) in each of the linear regression model and the no y-intercept model were very close in value. The same was observed for each of the HM (27.03082 vs. 27.14941) and AF (22.12397 vs. 22.12018) glycans.

### Example 8

This example demonstrates that the ADCC-TAF models and the ADCC-HM/AF models are interchangeable.

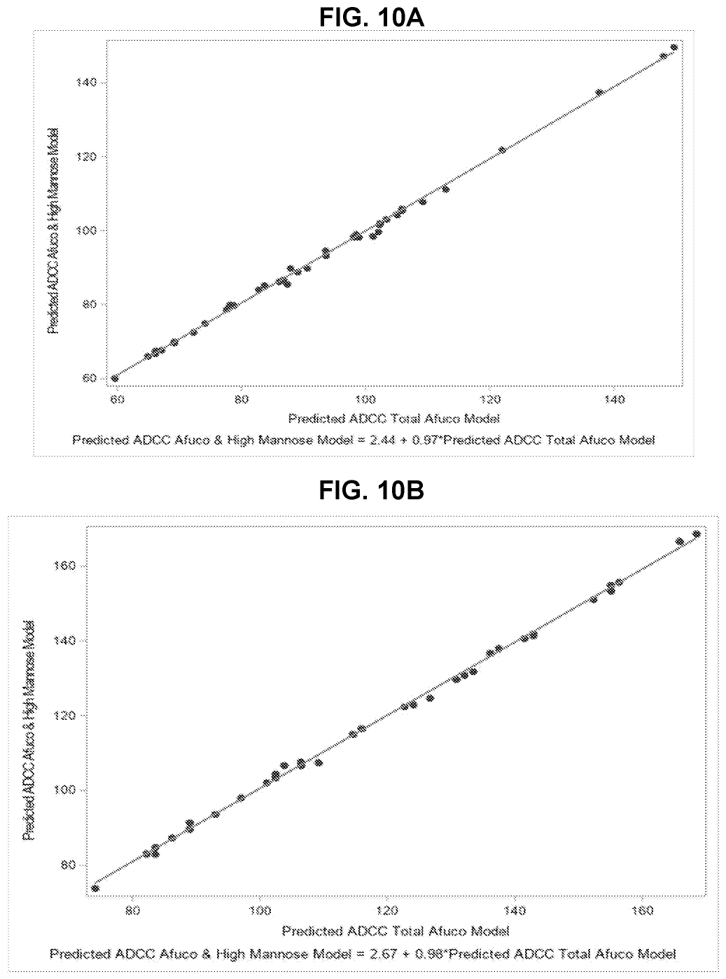

Equation 6 of Table 4, correlating ADCC to HM and AF glycan content, was used to calculate the predicted ADCC. The predicted ADCC was plotted against the predicted ADCC calculated according to Equation 5 of Table 4, which correlates ADCC to TAF glycan content. The results are graphed in FIG. 10A. The same steps were carried out for Equations 7 and 8 of Table 4 and graphed in FIG. 10B. The equation of the best fit line is provided below each graph. As shown in these figures and equations, the models are in high agreement with one another (p<0.0001). The slopes are nearly 1.0 (0.97 or 0.98). These data support that the ADCC of an antibody composition may be predicted based on one glycan type (TAF glycans) vs. two glycan types (HM and AF). Also, these data suggest that, for an antibody composition having a target ADCC, a target TAF may be calculated, and either HM or AF may be modified to achieve the target TAF. Methods of modifying HM or AF of an antibody composition is simpler than combining methods of modifying both HM and AF.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A method of determining product quality of an antibody composition, wherein the ADCC activity level of the antibody composition is a criterion upon which product quality of the antibody composition is based, said method comprising
i. determining the total afucosylated (TAF) glycan content of a sample of an antibody composition,
ii. determining the product quality of the antibody composition as acceptable and/or achieving the ADCC activity level criterion when the TAF glycan content determined in (i) is within a target range,
wherein the target range of TAF glycan content is based on (1) a target range of ADCC activity levels for a reference antibody and (2) a first model which correlates ADCC activity level of the antibody composition to the TAF glycan content of the antibody composition,
wherein the ADCC predicted by the first model is about 95% to about 105% of the ADCC predicted by a second model, wherein the second model correlates the ADCC activity level of the antibody composition to the high mannose (HM) glycan content and the afucosylated (AF) glycan content of the antibody composition.

2. The method of claim 1, wherein the ADCC predicted by the first model is about 100% of the ADCC predicted by the second model and/or the p-value of the first model is less than 0.0001 and/or the p-value of the second model is less than 0.0001.

3. (canceled)

4. The method of claim 1, wherein the ADCC activity level predicted by the first model is ˜12Q*% TAF, wherein Q is the number of antibody binding sites on the antigen to which the antibody binds and % TAF is the TAF glycan content of the antibody composition, optionally, wherein O is 1 or 2.

5. (canceled)

6. The method of claim 1, wherein the ADCC activity level predicted by the first model is ˜24*% TAF and/or the ADCC activity level predicted by the second model is ˜27*% HM+˜22*% AF, wherein % AF is the AF glycan content of the antibody composition and % HM is the HM glycan content of the antibody composition.

7. (canceled)

8. (canceled)

9. The method of claim 1, wherein the ADCC activity level predicted by the first model is ˜12*% TAF and/or the ADCC activity level predicted by the second model is ˜14.8*% HM+˜12.8*% AF.

10. (canceled)

11. The method of claim 1, wherein the reference antibody is rituximab or infliximab.

12. (canceled)

13. The method of claim 1, wherein the method is a quality control (QC) assay, optionally, an in-process QC assay.

14. (canceled)

15. The method of claim 1, wherein the sample is a sample of in-process material.

16. The method of claim 1, wherein the TAF glycan content is determined pre-harvest or post-harvest.

17. The method of claim 1, wherein the TAF glycan content is determined after a chromatography step, optionally, wherein the chromatography step comprises a capture chromatography, intermediate chromatography, and/or polish chromatography, and/or after a virus inactivation and neutralization, virus filtration, or a buffer exchange.

18. (canceled)

19. (canceled)

20. The method of claim 1, wherein the method is a lot release assay and/or wherein the sample is obtained from a manufacturing lot.

21. (canceled)

22. The method of claim 1, further comprising selecting the antibody composition for downstream processing, when the TAF glycan content is within a target range.

23. The method of claim 1, wherein, when the TAF glycan content determined in (i) is not within the target range, one or more conditions of the cell culture are modified to obtain a modified cell culture, optionally, wherein the method further comprises determining the TAF glycan content of a sample of the antibody composition obtained after one or more conditions of the cell culture are modified.

24. (canceled)

25. The method of claim 1, wherein, when the TAF glycan content determined in (i) is not within the target range, the method further comprises (iii) modifying one or more conditions of the cell culture to obtain a modified cell culture and (iv) determining the TAF glycan content of a sample of the antibody composition obtained from the modified cell culture, optionally, wherein, when the TAF glycan content determined in (i) is not within the target range, the method further comprises (iii) and (iv) until the TAF glycan content determined in (iv) is within the target range.

26. (canceled)

27. The method of claim 1, wherein an assay which directly measures ADCC activity of the antibody composition is carried out on the antibody composition only when the TAF glycan content is outside the target range, optionally, wherein the assay which directly measures ADCC activity is a cell-based assay that measures the release of a detectable reagent upon lysis of antigen-expressing cells comprising the detectable agent by effector cells that are bound to antibody binding both antigen-expressing and effector cells.

28. The method of claim 1, wherein an assay which directly measures ADCC activity of the antibody composition is not carried out on the antibody composition when the TAF glycan content is within the target range, optionally, wherein the assay which directly measures ADCC activity is a cell-based assay that measures the release of a detectable reagent upon lysis of antigen-expressing cells comprising the detectable agent by effector cells that are bound to antibody binding both antigen-expressing and effector cells.

29. (canceled)

30. A method of monitoring product quality of an antibody composition, comprising determining product quality of an antibody composition in accordance with a method of any one of the preceding claims, with a first sample obtained at a first timepoint and with a second sample taken at a second timepoint which is different from the first timepoint.

31. The method of claim 30, wherein each of the first sample and second sample is a sample of in-process material or the first sample is a sample of in-process material and the second sample is a sample of a manufacturing lot or the first sample is a sample obtained before one or more conditions of the cell culture are modified and the second sample is a sample obtained after the one or more conditions of the cell culture are modified.

32. (canceled)

33. (canceled)

34. A method of producing an antibody composition, comprising determining the product quality of the antibody composition, wherein product quality of the antibody composition is determined in accordance with a method of any one of the preceding claims, wherein the sample is a sample of in-process material, wherein, when the TAF glycan content determined in (i) is not within the target range, the method further comprises (iii) modifying one or more conditions of the cell culture to obtain a modified cell culture and (iv) determining the TAF glycan content of a sample of the antibody composition obtained from the modified cell culture, optionally, repeating steps (ii) and (iii) until the TAF glycan content is within the target range.

35. The method of claim 34, wherein one or more conditions of the cell culture are modified to primarily change the HM glycan content to achieve the target range of TAF glycan content or one or more conditions of the cell culture are modified to primarily change the AF glycan content to achieve the target range of TAF glycan content.

36. (canceled)
